(19) United States
(12) Patent Application Publication
(10) Pub. No.: US 2026/0365627 A1
(43) Pub. Date: Nov. 19, 2026

(54) NOVEL ECTONUCLEOTIDE PYROPHOSPHATASE / PHOSPHODIESTERASE 1 (ENPP-1) INHIBITORS AND USES THEREOF

(71) Applicant: SRAVATHI AI TECHNOLOGY PRIVATE LIMITED, Bangalore (IN)

(72) Inventors: Ganapathy Bhotla Venkata RAMANARAYANAN, Bangalore (IN); Vadivelu SARAVANAN, Bangalore (IN); Perumal SARAVANAN, Chittoor (IN); Unni AMBILI, Kongad (IN); Bharti NIVEDITA, Bengaluru (IN); Kulkarni NAGARAJ, Bengaluru (IN)

(21) Appl. No.: 19/099,052

(22) PCT Filed: Jul. 28, 2023
(86) PCT No.: PCT/IB2023/057701

§ 371 Date: Jan. 28, 2025

(30) Foreign Application Priority Data
Aug. 1, 2022 (IN) 202241044070

(51) Int. Cl. C07D 239/94; A61K 31/47; A61K 31/517; A61K 31/519; A61K 31/675; A61K 31/69; C07D 215/42; C07D 487/04; C07F 5/02; C07F 9/6512; C07F 9/6558

(52) U.S. Cl. CPC C07D 239/94; A61K 31/47; A61K 31/517; A61K 31/519; A61K 31/675; A61K 31/69; C07D 215/42; C07D 487/04; C07F 5/025; C07F 9/65128; C07F 9/65586

(57) ABSTRACT

The present invention discloses a novel type of highly potent ENPP-1 inhibitors for the treatment of various diseases particularly, cancer. These inventive inhibitors are small molecules. One of the novel structural features is incorporation of a sulfoximine-type moiety which is positioned between the tail and core parts. Method of treatment, pharmaceutical compositions and use thereof are also disclosed.

### FIELD OF THE INVENTION

The present disclosure relates to novel compounds of Formula I, which inhibit ENPP-1 protein and hence have the potential for use in immunotherapy for disease treatment. The invention also discloses synthetic methods for making the compounds, their pharmaceutical compositions, and potential uses in the treatment of many diseases particularly cancer.

### BACKGROUND OF THE INVENTION

One of the reported strategies in the treatment of cancer, particularly cancer immunotherapy is to potentiate anti-tumor immune responses of the human body. A recent journal publication, viz., Nat Cancer 1, 184-196 (2020), also reports that by inhibiting a protein known as Ectonucleotide Pyrophosphatase/Phosphodiesterase 1 (ENPP-1), which is known to negatively regulate innate immune signalling, thus enhancing the immune responses 2 as part of a treatment of cancer disease.

In journal publications, viz., a) J Hetmatol Oncol 13, 81 (2020) b) Molecules. 24(22). 4192 (2019) c) Cell Chemical Biology 27, 1347-1358 (2020), the role of cGAS-STING (cyclic GMP-AMP synthase-stimulator of interferon genes) pathway has been disclosed. The cGAS-STING pathway has a promising role in cancer immunotherapy as it is important in interferon (IFN) production and T cell priming. It now emerges that hydrolysis of cGAMP (cyclic GMP-AMP) by ENPP-1 attenuates cGAS-STING signaling. Therefore, inhibition of ENPP-1 would decrease hydrolysis of cGAMP resulting in enhancement of cGAS-STING signaling, with concomitant increase in the immune responses of the body. In a review article in Trends in Biochemical Science, 46(6), 446-460 (2021), it is pointed out that in addition to the crucial IFN signaling, cGAS-STING is much involved in autophagy.

ENPP-1 plays a regulatory function in immune cells such as neutrophils, macrophages, dendritic cells, natural killer cells, and B lymphocytes. ENPP-1 expression is heightened in M2 macrophages in the presence of cancer and promotes tumor growth and spread. The role of ENPP-1 in cancer is exemplified by the observations of enhanced tumor metastasis to the bone from breast cancer, for example, by over-expression of ENPP-1. ENPP-1 belongs to the family of Phosphodiesterase. A patent publication, WO2018119328A 1 reviews the roles and types of different phosphodiesterases. Phosphodiesterases comprise a class of enzymes that catalyze the hydrolysis of a phosphodiester bond. In some instances, phosphodiesterase has been linked with viral infection and its inhibition has been correlated with a reduction in viral replication. In some instances, the class of phosphodiesterase further comprises cyclic nucleotide phosphodiesterase, phospholipases C and D, autotaxin, sphingomyelin phosphodiesterase, restriction endonucleases, and small-molecule phosphodiesterases. Literature J Biol Chem 280(24), 22962(2005) and Biochemistry Moscow 75, 1-6 (2010) supports the molecular functions with chemical similarity between phosphodiesterase, nuclease (DNases, RNases), nucleotidase, phosphatase. In another instance, phosphodiesterase is linked with a bacterial infection, e.g., an infection from a Gram-negative bacterium or a Gram-positive bacterium. In some cases, the bacterium is Listeria monocytogenes, Mycobacterium tuberculosis, Francisella novicida, Legionella pneumophila, Chlamydia trachomatis, Streptococcus pneumoniae, or Neisseria gonorrhoeae. Hence inhibitors of phosphodiesterases would potentially impact treatment of many diseases. Inhibitors which have high specificity in inhibiting a particular phosphodiesterase, for example ENPP-1, are much needed in the industry.

There are several prior art reports which disclose ENPP-1 inhibitors with different structures of varied potency. The key features of the recently reported references of prior art are: a) ENPP-1 inhibitors predominantly target cancer diseases b) structures of the inhibitors possess at least three basic components comprising a tail, a core and zinc binding domain (domain is also referred as part), as exemplified in formulae II-X and c) some of these structures have linking groups (linkers) between the core and zinc binding part and/or some structures have linkers between the core and tail parts.

Structures of compounds of certain prior art inventions relevant to ENPP-1 inhibition are highlighted in Scheme 1. Though the scaffolds represented in formulae II-V are not exactly similar, they contain tail, core, and zinc binding parts. Some of the scaffolds have only one linker and others two linkers. ENPP-1 inhibitory activity of the compounds of structures of formulae II-V is disclosed in the respective literature references for cancer treatment.

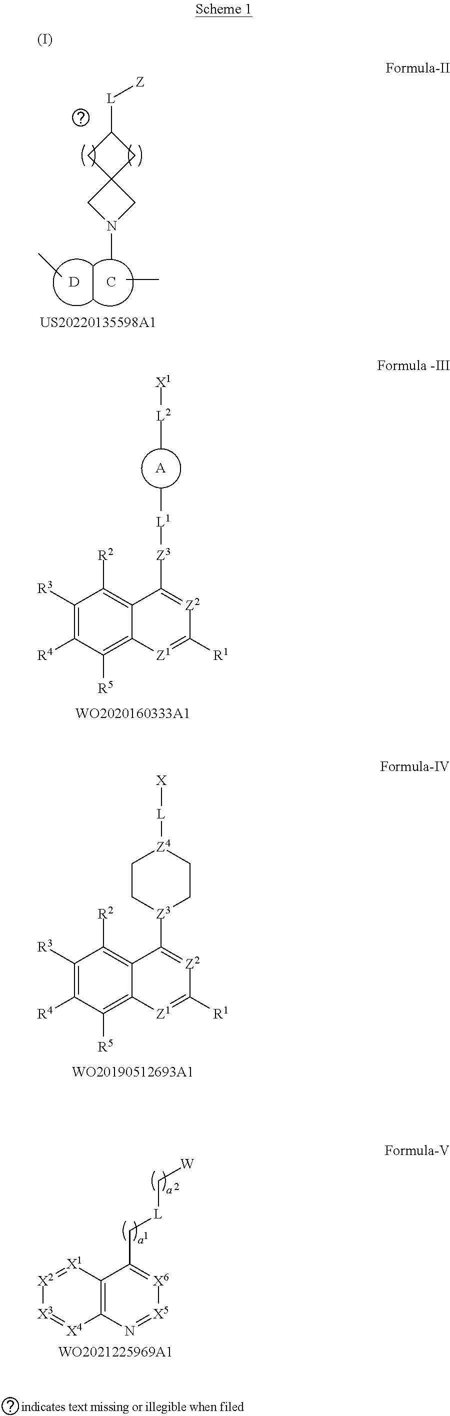

Patent publication, US20220135598A 1 discloses inhibitor structure conforming to the Formula II where tail part is primarily quinoline or substituted quinoline moiety with specific substitutions on the quinoline ring, core part is a cyclic ring or fused spiro ring; linker part L which links the core part with the zinc binding part can be a bond, linear or branched C1-C6 alkylene or linear or branched C2-C6 alkenylene; and zinc binding part is —NRcS(O)2NH2, —NRcS(O)2CH3, —SO2NH2, —NRcC(O)CH3, —C(O)OH, —CONH2, NRcCONH2, —CONH(OH), —B(OH)2, —P(O)(OH)2, —SO20H, —NRcS(O)2CF3, —NRcS(O)2NHCH3, or —NR1CH2C6-aryl-S(O)2NH2. The Rc is defined as hydrogen, alkyl, substituted cycloalkyl, substituted alkylene and substituted heteroaryl.

Patent publication WO2020160333A1 discloses inhibitor structure conforming to the Formula III, where the presence of an additional linker L¹ between core part and tail part is a key feature. The linker L2 is present between the core and zinc binding domain. Zinc binding group has been disclosed as phosphorus-containing group or urenyl. The core part could be aryl. Further, linker L1 is explored as alkyl, alkenylene, alkynylene, arylene, aralkylene and linking moieties containing functional group including without limitation: amido, ureylene, imide, epoxy, epithio, epidioxy, cabonyldioxy, alkyldioxy, epoxyimino, epimino, and carbonyl.

Patent publication, WO2019051269A1 discloses a structure as depicted in Formula-N, wherein linker between core and X was termed as L, which can be a (C1-6)alkyl linker or a substituted (C1-6)alkyl linker, optionally substituted with a heteroatom or linking functional group, such as an ester (—CO2-), amido (—CONH), carbamate (—OCONH), ether (—O—), thioether (—S—) and/or amino group. The tail part is based on quinazoline moiety and core part represented as C is an aromatic ring. The zinc binding part represented as X was based on phosphoric acid or sulfonamide or ureylene moieties.

Patent publication, WO2021225969A1 discloses a structure as depicted in FIGURE-V, where L is a bond, —O—, —C(O)—, —NR6c-, or —OCR7c-*, wherein * represents the point of attachment containing zinc binding part. W in FIGURE-V contains both the core group and zinc binding part connected through the linker L. The core group is an aryl or heteroaryl moiety. The zinc binding part is a sulfoximine moiety with only a hydrogen atom as the substituent on the nitrogen atom of the sulfoximine group. The R6c, R7c are each independently hydrogen or C1-3 alkyl wherein a1 and a2 are independently 0, 1, 2 or 3.

Patent publication, WO2021226136A1 discloses a structure akin to the one depicted in Formula-N for ENPP-1 inhibition.

Patent publication, WO2019046778A1 discloses a structure as depicted in Formula-VI, where there are two linkers named L and L1 and with a sulfonamide type end group for ENPP-1 inhibition. Where X is —NR7-, —O—, —S—, —S(═O)—, —S(β)2-, or —CR8R9-; L is a bond or —CR10R11-; and L1 is a bond or —CR13R14-. R7 is hydrogen, —CN, substituted alkoxy, substituted ester, substituted carbonyl, substituted amide, substituted sulfoxide, substituted sulfone, optionally substituted alkyl, alkenyl, alkynyl, cycloalkyl, heterocycloalkyl, aryl, heteroaryl. R8 and R9 are independently hydrogen, deuterium, halogen, —CN, substituted alkoxy, —NO₂, substituted amino, optionally substituted alkyl, alkenyl, alkynyl, cycloalkyl, heterocycloalkyl.

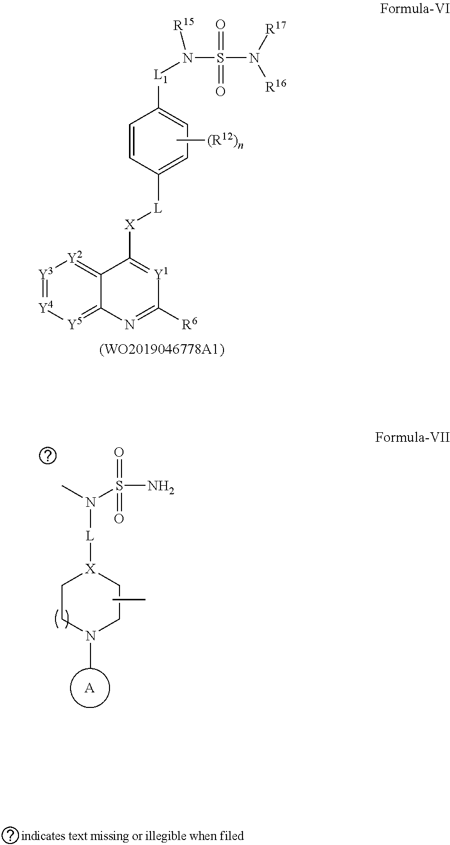

Patent publication, US20190282703A1 discloses a structure as depicted in Formula-VII, where there is single linker, L as specified and a sulfonamide end group and a monocyclic tail group. L is defined as —(CR3R4)n- where X is —N— or —CH—. R3 and R4 on the same carbon are taken together to form an oxo.

Patent publication, WO2021158829A1 discloses a structure as depicted in Formula VIII where L is selected from the group consisting of an C1-C5 alkyl, and C1-C5 alkenyl; and where Y is selected from the group consisting of —CR4R5-, —NR6-, —N(CH2)mO—, —O—, —S—, —S(O)—, —S(O)2, aryl, and heteroaryl; wherein m is 2 or 3 for treatment of cancer, bacterial or viral diseases. R4, R5 and R6 are independently selected from the group consisting of hydrogen and lower alkyl; or an isomer, hydrate, solvate, polymorph, tautomer or a pharmaceutically acceptable salt thereof.

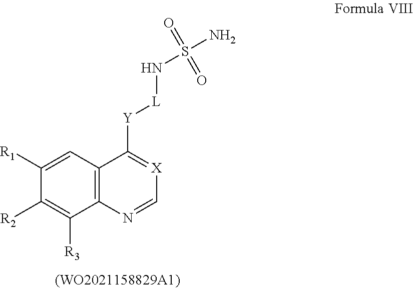

Patent publication, WO2020140001A1 discloses quinazoline based structure as depicted in Formulae-IX that inhibit ENPP-1 enzymatic activity and are therefore useful for the treatment of diseases.

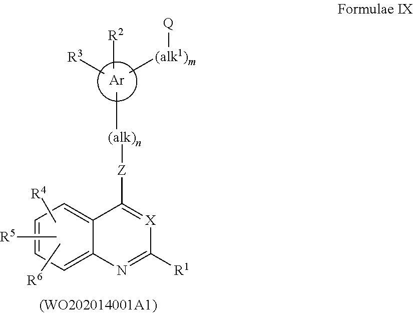

Other related prior art references which disclose certain compounds as ENPP-1 inhibitors of structures not falling in the tail-core-zinc binding part description given in FIGURES II-IX are mentioned below.

WO2022056068A1 discloses small molecule ENPP-1 inhibitors for treating a variety of cardiac conditions. Patent publication, WO2019023635A 1 discloses substituted -3H-imidazo[4,5-c]pyridine and 1H-pyrrolo[2,5-c]pyridine series of novel ENPP-1 inhibitors and stimulator for interferon genes (sting) modulator as cancer immune therapeutics. A patent publication, WO2021053507A 1 discloses 2-amino-S6-substituted thiopurine compounds as inhibitors of the ENPP-1 for treatment of cancer, infectious disease, and other conditions associated.

Additionally, ENPP-1 inhibitors play a role in DNA damage repair process (See reference US20220135598A1). The patent publications, WO2019023635A1, WO2021158829A1 mentions that ENPP-1 is an attractive druggable target for the development of novel anticancer, cardiovascular, diabetes, obesity and anti-fibrotic therapeutics. Patent publications, WO2022056068A1, WO2019046778A1 mentions that modulators of ENPP-1 may also be useful against bacteria and fungi.

In a patent publication, WO2022125613A1, certain phosphonates have been shown as inhibitors of not only ENPP-1 but also CdnP. Cyclic di-nucleotide phosphodiesterase (CdnP, also known as Rv2837c) is a phosphodiesterase in regulating cyclic dinucleotide signaling during intracellular infections of M. tuberculosis. The structure of the phosphonates disclosed in '613 patent application also can be classified as tail-core-zinc binding domain and may be represented as Formula X.

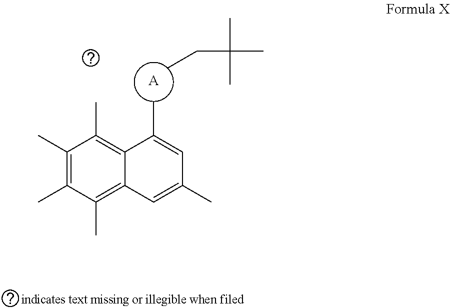

The main therapeutic use or purpose behind ENPP-1 inhibitors is to provide treatment to patients or subjects suffering from cell proliferative diseases and cancers including, without limitation, glioma, glioblastoma multiforme, paraganglioma, supratentorial primordial neuroectodermal tumours, acute myeloid leukemia (AML), prostate cancer, thyroid cancer, colon cancer, chondrosarcoma, cholangiocarcinoma, peripheral T-cell lymphoma, melanoma, intrahepatic cholangiocarcinoma (IHCC), myelodysplastic syndrome (MDS), myeloproliferative disease (MPD), and other solid tumours. The non-availability of targeted treatments for these cancers and cell proliferative diseases is driving the current continued search of therapeutic agents, particularly new ENPP-1 Inhibitors that could show selectivity to some of these adverse conditions and diseases.

The brief overview given above on structures with tail-core-zinc binding group describes scaffolds of potentially high potency for inhibition of ENPP-1 to boost immune activity. However, none of these structures seem to be clinically proven. The shortcomings of these structures are not clear from the literature reports. Newer structures are still required to address the dire need to fight different types of cancer or carcinoma. Accordingly, the present invention discloses a novel structure for highly efficacious molecules for use against one or more types of maladies.

### SUMMARY OF THE INVENTION

The present disclosure relates to compounds of Formula I, which are potent inhibitors of ENPP-1 protein and hence have the potential for use as for example, in immune therapy for disease treatment. The invention also discloses synthetic methods for making these compounds and invitro bioactivity results to indicate the potential use of these inhibitors in treatment of various diseases including cancer. Specifically, the invention discloses a compound of Formula I or a pharmaceutically acceptable salt, hydrate, solvate, tautomer, isomer thereof.

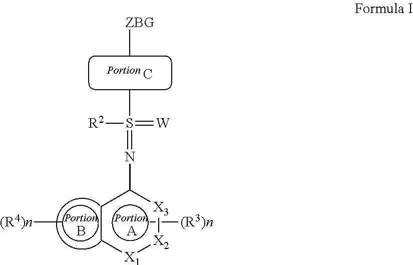

wherein the Formula I comprises portions A and B together constituting tail part, a portion C constituting core part, a sulfoximine-type group R2-S(═W)(═N) which links the tail part with the core part and a zinc binding group (ZBG), where,
the portion A is a six-membered aryl or heteroaryl, which is optionally substituted with one or more R³ groups and where X₁, X₂ and X₃ is CH, N, CR′ with the proviso that not more than two of X₁, X₂ and X₃ is simultaneously N;
the portion B is C6 aryl or 5-6 membered heteroaryl, optionally substituted with one or more R4 groups, wherein the portion B is fused to the portion A where the two shared atoms between the portions B and A come from a pair of carbon atoms or from a pair of atoms where one of the atoms is nitrogen and the other is carbon;
the portion C is selected from the group of structures consisting of (i), (ii), (iii), (iv), (v) and (vi),
where L1 is connected to (i) at variable positions of the ring
   where E1 is connected to the sulphur atom of the R2-S(═W)(═N) group and linker (L1) is connected to “a” atom of the zinc binding group (ZBG)

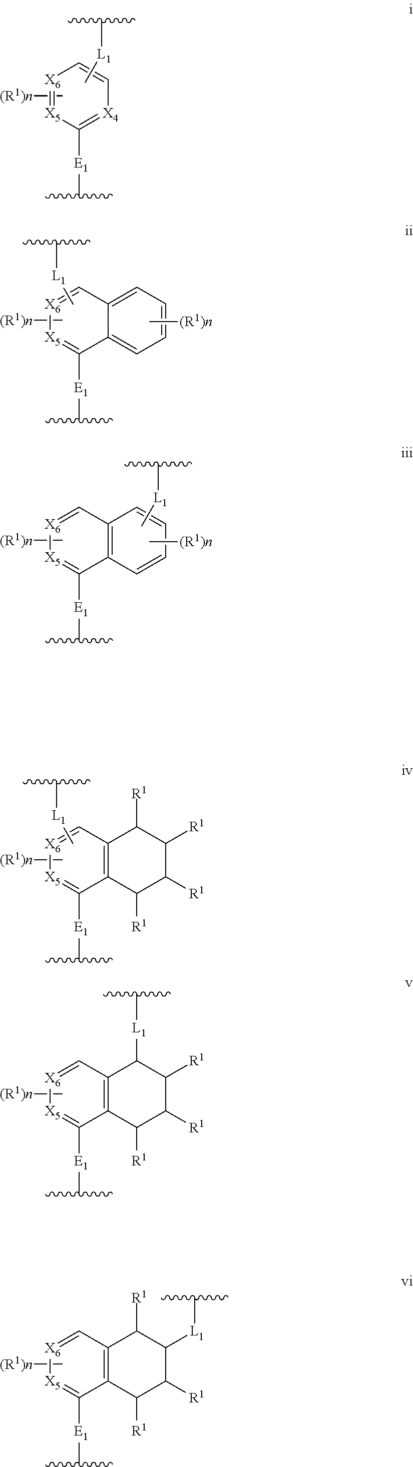

n=0,1,2,3,4 with the proviso that when n>1, the R¹ groups may be the same or different,
R¹, R³, R⁴ groups are selected from the group consisting of R′, halo, OR′, OAr, SR′, SAr, NHAr, NR‘R’, CN, SCN, —NHCOR′, COR′, COOR′, COOAr, CF₃, CHF₂, CH₂F, OCF₃, SCF₃ and CH₂Ar, where R′═H, CN, C1-6 straight chain alkyl, branched chain alkyl, cycloalkyl, CH₂Ar and where Ar=aryl, substituted aryl, heteroaryl, or substituted hetero aryl;
E1=—(CH₂)_{y}— where y=0.1 or 2;
L1=—(CH₂)_{y1}, NR″, O or S, C1-6 straight chain alkyl, branched chain alkyl, —(CH₂)_{y1} cycloalkyl, where y1=0,1, 2, or 3; Wherein R″═H, C1-6 straight chain alkyl, branched chain alkyl, cycloalkyl, aryl, substituted aryl, halogenated alkyl;
   X₄, X₅ and X₆═CH, N or CR′;
the portion A is connected to nitrogen atom of the R2-S(═W)(═N) group
   where S is chiral sulphur atom,
   N is nitrogen,
   W is O or NH;
   R2 is selected from the group consisting of C1-6 straight, branched, cycloalkyl, alkenyl, alkylene, alkynyl, halo, aryl, heteroaryl, heterocycle, substituted aryl, CF₃, CHF₂, CH₂F, CN and a 2 to 6 membered alkylene group, one end of which is bonded to the sulphur atom and the other end bonded to portion C at the position alpha to the carbon atom of portion C which is bonded to sulphur atom when y=0, forming a cyclic structure and
ZBG as shown in structure (vii)

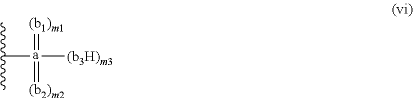

where a=S, P, C, or B atom;
b1, b2=O, S, NH or CH₂,
b3=O, S, NH, CH₂ or NH₂O
and m₁, m₂ and m₃=0, 1 with the proviso that only one of m₁, m₂ and m₃ can be 0 at a given instance

In one aspect, a general method of preparation of compounds and intermediates required for synthesis of compound of Formula I is disclosed.

The compound represented by formula I, inhibits function of phosphodiesterase enzyme which is selected from a group consisting of ENPP-1, cyclic nucleotide phosphodiesterase, phospholipases C and D, autotaxin, sphingomyelin phosphodiesterase, DNases, R ases, restriction endonucleases, and small-molecule phosphodiesterases.

The isomer of compound I is selected from a group consisting of stereoisomer of the Formula I and positional isomer arising from linkage of sulfoximine-type fragment of the formula I.

The stereoisomer is selected from a group consisting of (R) isomer of the formula I, (S) isomer of the Formula I and a combination thereof.

The positional isomer of Formula I is selected from a group consisting of a molecule where the core and the tail parts of the Formula I are bonded to the sulfoximine-type group at the sulphur and the nitrogen atoms respectively and a molecule where the core and the tail parts of the Formula I are bonded to the sulfoximine-type group at the nitrogen and the sulphur atoms respectively.

Invitro assay results point to the potential of these compounds in inhibiting phosphodiesterase such as ENPP-1 and hence in treatment of diseases such as cancer.

### DETAILED DESCRIPTION

It is to be understood that the terminology used herein is for the purpose of describing specific embodiments only and is not intended to limit the scope of the present invention. In this specification and in the claims that follow, reference will be made to the terms that shall be defined to have the following meanings.

### DEFINITIONS

As used herein unless otherwise specified, “alkyl” refers to a monovalent saturated aliphatic hydrocarbyl group having from 1 to 14 carbon atoms and, in some embodiments, from 1 to 6 carbon atoms. The term “alkyl” includes, by way of example, linear and branched hydrocarbyl groups such as methyl (CH3-), ethyl (CH3CH2-), n-propyl (CH3CH2CH2-), isopropyl ((CH3)2CH—), n-butyl (CH3CH2CH2CH2-), isobutyl ((CH3)2CHCH2-), sec-butyl ((CH3)(CH3CH2)CH—), t-butyl ((CH3)3C—), n-pentyl(CH3CH2CH2CH2CH2-), and neopentyl ((CH3)3CCH2-).

“Cycloalkyl” refers to a saturated or partially saturated cyclic group of from 3 to 14 carbon atoms and no ring heteroatoms and having a single ring or multiple rings including fused, bridged, and spiro ring systems. For multiple ring systems having aromatic and non-aromatic rings that have no ring heteroatoms, the term “cycloalkyl” applies when the point of attachment is at a non-aromatic carbon atom (e.g., 5,6,7,8-tetrahydronaphthalene-5-yl). The term “Cycloalkyl” includes cycloalkenyl groups, such as cyclohexenyl. Examples of cycloalkyl groups include, for instance, adamantyl, cyclopropyl, cyclobutyl, cyclohexyl, cyclopentyl, cyclooctyl, cyclopentenyl, and cyclohexenyl. Examples of cycloalkyl groups that include multiple bicycloalkyl ring systems are bicyclohexyl, bicyclopentyl, bicyclooctyl, and the like.

“Aryl” refers to an aromatic group of from 5 to 14 carbon atoms and no ring heteroatoms and having a single ring (e.g., phenyl) or multiple condensed (fused) rings (e.g., naphthyl or anthryl). For multiple ring systems, including fused, bridged, and spiro ring systems having aromatic and non-aromatic rings that have no ring heteroatoms, the term “Aryl” or “Ar” applies when the point of attachment is at an aromatic carbon atom (e.g., 5,6,7,8 tetrahydronaphthalene-2-yl is an aryl group as its point of attachment is at the 2-position of the aromatic phenyl ring).

“Alkenyl” as used herein refers to an unsaturated linear or branched univalent hydrocarbon chain or combination thereof, having at least one site of olefinic unsaturation (i.e., having at least one moiety of the formula C═C) and having the number of carbon atoms designated (i.e., C₂-C₁₀ means two to ten carbon atoms). The alkenyl group may be in “cis” or “trans” configurations, or alternatively in “E” or “Z” configurations. Preferred alkenyl groups are those having 2 to 20 carbon atoms (a “C₂-C₂M alkenyl”), having 2 to 8 carbon atoms (a “C₂-C₈ alkenyl”), having 2 to 6 carbon atoms (a “C₂-C₆ alkenyl”), or having 2 to 4 carbon atoms (a “C₂-C₄ alkenyl”) Examples of alkenyl include, but are not limited to, groups such as ethenyl (or vinyl), prop-1-enyl, prop-2-enyl (or allyl). 2-methylprop-1-enyl, but-1-enyl, but-2-enyl, but-3-enyl, buta-1,3-dienyl, 2-methylbuta-1,3-dienyl, homologs and isomers thereof, and the like.

“Alkylene” as used herein refers to the same residues as alkyl but having bivalency. Preferred alkylene groups are those having 1 to 6 carbon atoms (a “C₁-C₆ alkylene”), 1 to 5 carbon atoms (a “C₁-C₅ alkylene”), 1 to 4 carbon atoms (a “C₁-C₄ alkylene”) or 1 to 3 carbon atoms (a “C₁-C₃ alkylene”). Examples of alkylene include, but are not limited to, groups such as methylene (—CH₂—), ethylene (—CH₂CH₂—), propylene (—CH₂CH₂CH₂—), butylene (—CH₂CH₂CH₂CH₂—), and the like.

“Alkynyl” as used herein refers to an unsaturated linear or branched univalent hydrocarbon chain or combination thereof, having at least one site of acetylenic unsaturation (i.e., having at least one moiety of the formula C≡C) and having the number of carbon atoms designated (i.e., C₂-C₁₀ means two to ten carbon atoms). Preferred alkynyl groups are those having 2 to 20 carbon atoms (a “C₂-C₂M alkynyl”), having 2 to 8 carbon atoms (a “C₂-C₅ alkynyl”), having 2 to 6 carbon atoms (a “C₂-C₆ alkynyl”), or having 2 to 4 carbon atoms (a “C_{c2}-C₄ alkynyl”). Examples of alkynyl include, but are not limited to, groups such as ethynyl (or acetylenyl), prop-1-ynyl, prop-2-ynyl (or propargyl), but-1-ynyl, but-2-ynyl, but-3-ynyl, homologs and isomers thereof, and the like.

“Halo” or “halogen” refers to elements of the Group 17 series having atomic number 9 to 85. Preferred halo groups include fluoro, chloro, bromo and iodo. Where a residue is substituted with more than one halogen, it may be referred to by using a prefix corresponding to the number of halogen moieties attached, e.g., dihaloaryl, dihaloalkyl, trihaloaryl etc. refer to aryl and alkyl substituted with two (“di”) or three (“tri”) halo groups, which may be but are not necessarily the same halo; thus 4-chloro-3-fluorophenyl is within the scope of dihaloaryl. An alkyl group in which each hydrogen is replaced with a halo group is referred to as a “perhaloalkyl.” A preferred perhaloalkyl group is trifluoroalkyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃).

“Heteroaryl” refers to and includes unsaturated aromatic cyclic groups having from 1 to 10 annular carbon atoms and at least one annular heteroatom, including but not limited to heteroatoms such as nitrogen, oxygen and sulfur, wherein the nitrogen and sulfur atoms are optionally oxidized, and the nitrogen atom(s) are optionally quaternized. A heteroaryl group can be attached to the remainder of the molecule at an annular carbon or at an annular heteroatom. Heteroaryl may contain additional fused rings (e.g., from 1 to 3 rings), including additionally fused aryl, heteroaryl, cycloalkyl, and/or heterocyclyl rings. Examples of heteroaryl groups include, but are not limited to imidazolyl, pyrrolyl, pyrazolyl, 1,2,4-triazolyl, thiophenyl, furanyl, thiazolyl, isothiazolyl, 1,3,4-thiadiazolyl oxazolyl, isoxazolyl, 1,3,4-oxadiazolyl, pyridyl, pyrimidyl, pyridazinyl, pyrazinyl, indolyl, indazolyl, benzoimidazolyl, pyrrolopyridinyl, pyrrolopyridazinyl, pyrrolopyrimidinyl, pyrazolopyridinyl, pyrazolopyrimidinyl, imidazopyridinyl, purinyl, benzofuranyl, furopyridinyl, benzooxazolyl, benzothiophenyl, benzothiazolyl, oxazolopyridinyl, thiazolopyridinyl, thienopyridinyl, quinolinyl, quinolonyl, naphthyridinyl, quinazolinyl, pyridopyrimidinyl, cinnolinyl or pyridopyridazinyl and the like.

“Heterocycle” or “heterocyclyl” refers to a saturated or an unsaturated non-aromatic group having from 1 to 10 annular carbon atoms and from 1 to 4 annular heteroatoms, such as nitrogen, sulfur or oxygen, and the like, wherein the nitrogen and sulfur atoms are optionally oxidized, and the nitrogen atom(s) are optionally quaternized. A heterocyclyl group may have a single ring or multiple condensed rings. A heterocycle comprising more than one ring may be fused, spiro or bridged, or any combination thereof. In fused ring systems, one or more of the fused rings can be aryl or heteroaryl. Examples of heterocyclyl groups include, but are not limited to, aziridinyl, azetidinyl, oxetanyl, morpholinyl, thiomorpholinyl, azepanyl tetrahydropyranyl, dihydropyranyl, piperidinyl, piperazinyl, pyrrolidinyl, thiazolinyl, thiazolidinyl, tetrahydrofuranyl, tetrahydrothiophenyl, and the like.

“Optionally substituted” unless otherwise specified means that a group may be unsubstituted or substituted by one or more (e.g., 1, 2, 3, 4 or 5) of the substituents listed for that group in which the substituents may be the same or different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 2 to 5, 3 to 5, 2 to 3, 2 to 4, 3 to 4, 1 to 3, 1 to 4 or 1 to 5 substituents.

A “medicament” or “pharmaceutical composition” refers to a pharmaceutical formulation in administrable form comprising at least one pharmaceutically active ingredient and one or more pharmaceutically acceptable carrier.

A “pharmaceutically acceptable carrier” or “pharmaceutically acceptable excipient” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” or “treating” is an approach for obtaining beneficial or desired results including clinical results. For example, beneficial or desired results include, but are not limited to, one or more of the following: decreasing symptoms resulting from the disease, increasing the quality of life of those suffering from the disease, decreasing the dose of other medications required to treat the disease, delaying the progression of the disease, and/or prolonging survival of individuals. In reference to cancers or other unwanted cell proliferation, beneficial or desired results include shrinking a tumor (reducing tumor size); decreasing the growth rate of the tumor (such as to suppress tumor growth); reducing the number of cancer cells; inhibiting, retarding or slowing to some extent and preferably stopping cancer cell infiltration into peripheral organs; inhibiting (slowing to some extent and preferably stopping) tumor metastasis; inhibiting tumor growth; preventing or delaying occurrence and/or recurrence of tumor; and/or relieving to some extent one or more of the symptoms associated with the cancer. In some embodiments, beneficial or desired results include preventing or delaying occurrence and/or recurrence, such as of unwanted cell proliferation.

As used herein, “delaying development of a disease” means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late-stage cancer, such as development of metastasis, may be delayed.

As used herein, an “effective dosage” or “effective amount” of compound or salt thereof or pharmaceutical composition is an amount sufficient to effect beneficial or desired results.

As used herein, the term “individual” is a mammal, including humans. An individual includes, but is not limited to, human, bovine, horse, feline, canine, rodent, or primate. In some embodiments, the individual is human. The individual (such as a human) may have advanced disease or lesser extent of disease, such as low tumor burden. In some embodiments, the individual is at an early stage of a proliferative disease (such as cancer). In some embodiments, the individual is at an advanced stage of a proliferative disease (such as an advanced cancer).

In some aspects, sarcomas and carcinomas are cancer that may be treated as solid tumours whereas leukemia are the cancer that may be treated as liquid tumours. Present invention may treat different types of cancers that include, but are not limited to, adrenocortical cancer, bladder cancer, brain tumours, breast cancer, prostate cancer, colorectal cancer, colon cancer, endometrial cancer, gallbladder cancer, gastric cancer, head and neck cancer, hematopoietic cancer, kidney cancer, leukemia, oral cancer, uterine carcinoma, hodgkin lymphoma, liver cancer, lung cancer, pancreatic cancer, ovarian cancer, sarcoma, skin cancer and thyroid cancer. The breast cancer is classified as carcinoma of breast (ER negative or ER positive), mammary adenocarcinoma, primary breast ductal carcinoma, mammary ductal carcinoma (ER positive, ER negative or HER2 positive), triple negative breast cancer (TNBC), HER2 positive breast cancer or luminal breast cancer. The breast cancer is unclassified. In some cases, a basal-like TNBC, an immunomodulatory TNBC, mesenchymal TNBC (mesenchymal or mesenchymal stem-like) or a luminal androgen receptor TNBC are triple negative breast. In some embodiments, prostate adenocarcinoma is prostate cancer. Other therapeutic use of the compounds including the ovary adenocarcinoma, lung carcinoma, adenocarcinoma, non-small lung carcinoma, mucoepidermoid, anaplastic large cell cancer, the colon adenocarcinomas, colon carcinoma, metastatic colorectal cancer, colon adenocarcinoma, astrocytoma, glioblastoma, meduloblastoma, neuroblastoma or meningioma, stomach cancer, cholangiocarcinoma or hepatoblastoma, hepatocellular carcinoma, liver cancer, medullary thyroid cancer or follicular thyroid cancer, papillary thyroid carcinomas, uterine papillary serous carcinoma or uterine clear cell carcinoma, gallbladder adenocarcinoma or squamous cell gallbladder carcinoma, renal cell carcinoma or urothelial cell carcinoma, adrenal cortical carcinoma, fibrosarcoma or Ewing's sarcoma, osteosarcoma, rhabdomyosarcoma, synovial sarcoma, basal cell carcinoma, melanoma or squamous carcinoma, cancer of the trachea, laryngeal cancer, nasopharyngeal cancer and oropharyngeal cancer, acute lymphoblastic leukemia, acute promyelocytic leukemia, chronic myelogenous leukemia, chronic lymphocytic leukemia, mantle cell lymphoma or multiple myeloma.

### Molecular Biology of ENPP-1

Ectonucleotide pyrophosphatase/phosphodiesterase family member 1 (ENPP-1) is a 925 amino acid length protein having a molecular mass of 104924 Da. This protein is predominantly found in the extracellular space, lysosomal membrane and in the plasma membrane. The ENPP-1 protein which belongs to the nucleotide pyrophosphatase/phosphodiesterase family is a homodimer that requires zinc ion as a cofactor for eliciting biological function. The molecular functions reported for ENPP-1 protein are nucleic acid binding, exonuclease activity, phosphodiesterase I activity, 3′-phosphoadenosine 5′-phosphosulfate binding, ATP binding, calcium ion binding, cyclic-GMP-AMP hydrolase activity, dTTP diphosphatase activity, exonuclease activity, insulin receptor binding, NADH pyrophosphatase activity, nucleic acid binding, nucleoside-triphosphate diphosphatase activity, nucleotide diphosphatase activity, phosphodiesterase I activity, polysaccharide binding, protein homodimerization activity, scavenger receptor activity, zinc ion binding and nucleotide diphosphatase activity. ENPP-1 protein is involved in the hydrolysis of ATP, GTP, CTP, TTP and UTP to their respective monophosphates with release of pyrophosphate and diadenosine polyphosphates. The involvement of ENPP-1 protein is identified in several biological processes such as generation of precursor metabolites, metabolism of phosphate containing compounds, regulation of the availability of nucleotide sugars in the endoplasmic reticulum and Golgi, regulation of purinergic signalling, endocytosis, immune responses, and nucleoside triphosphate catabolic process. One of the critical functions of ENPP-1 is reported to be the hydrolysis of 2′,3′-cGAMP (cyclic GMP-AMP), a second messenger that activates TMEM 173/STING. The hydrolysis of cyclic GMP-AMP leads to the reduced expression of STING pathway downstream components, which are crucial for the maintenance of immune functions. Hence, ENPP-1 mediated STING pathway inactivation leads to immune suppression and enhanced tumor cell metastasis.

ENPP-1 belongs to the class of Phosphodiesterases. Phosphodiesterases comprise a class of enzymes that catalyze the hydrolysis of a phosphodiester bond. In some instances, phosphodiesterase has been linked with viral infection and its inhibition has been correlated with a reduction in viral replication.

In some instances, the class of phosphodiesterases further comprises cyclic nucleotide phosphodiesterase, phospholipases C and D, autotaxin, sphingomyelin phosphodiesterase, DNases, RNases, restriction endonucleases, and small-molecule phosphodiesterases.

In another instance, phosphodiesterase is linked with a bacterial infection, e.g., an infection from a Gram-negative bacterium or a Gram-positive bacterium. In some cases, the bacterium is Listeria monocytogenes, Mycobacterium tuberculosis, Francisella novicida, Legionella pneumophila, Chlamydia trachomatis, Streptococcus pneumoniae, or Neisseria gonorrhoeae. Hence inhibitors of phosphodiesterases would potentially impact treatment of many diseases.

Crystallographic Information on Protein-Ligand Complex 6 crystal structures are reported for ENPP-1 protein. 2YS0, 6WET, 6WEU, 6WEV, 6WEW and 6WFJ are the reported PDB codes for ENPP-1.Out of the available PDB structures 6WEV was considered for in-silico studies. The resolution of this protein was reported to be 2.90 Å. 6WEV is the target considered for the execution of in-silico studies. The ENPP-1 protein complexed with N-{[1-(6,7-dimethoxy-5,8-dihydroquinazolin-4-yl) piperidin-4-yl]methyl}sulfuric diamide (PDB ID: 6WEV) has additional cofactors such as 2-acetamido-2-deoxy-beta-D-glucopyranose, phosphate ion, calcium ion and zinc ions. Zinc ions play essential role in the catalytic activation of ENPP-1. Hence the interaction of drug candidate molecules with zinc ions is critical for eliciting enzyme inhibition.

### Molecular Docking—Methodology

Molecular docking studies were executed by AutoDock4Zn program, which incorporates improved force field parameters for addressing the co-ordination properties of zinc ions with the small molecules. The energetic and geometric components of zinc ion interactions with small molecules are captured in this program. Traditional autodock force field accounts for van der Waals, hydrogen bond, Coulomb electrostatic, desolvation and ligand torsional entropy terms for describing the interactions between ligand and receptor. But the traditional autodock forcefield is inadequate for handling zinc ions as van der Waals equilibrium distances for the atoms involved in zinc coordination are significantly larger than the coordination distances and lack of specific terms for metal co-ordination. Hence potential energy term associated with the pairwise interactions of each atom type involved in zinc ion co-ordination is added to the current autodock forcefield.

### Molecular Docking Results

The structural features associated with ENPP-1 inhibitors are categorized as zinc binding head or group, core, and tail part. Out of the three parts, the presence of zinc binding head plays vital role in the ENPP-1 inhibition as this group co-ordinates with the zinc ion present in catalytic site of enzyme. The core and tail parts anchor the compound tightly in the binding pocket. The inhibitor design was initiated by considering these 3 structural elements. ENPP-1 co-crystalized with the inhibitor reported to have good potency was considered for the docking studies. The active site residues of ENPP-1 protein include D218, F257, L290, K295, D326, S325, K338, W322, F321, Y371, Y340, P323, T356, D376, H380 and Zn ions. The re-docking of the inhibitor to the binding pocket of enzyme was performed to visualize the binding profile. Prior art available on ENPP-1 docking studies emphasize that the closeness of zinc binding head of inhibitor to zinc atoms present in the catalytic site results in the higher degree of enzyme inhibition. This observation was taken into consideration for the redocking studies. The zinc binding head of the reference compound was found to have a close association with the zinc ions present in the active site.

### Inhibitors of ENPP-1

The inventive compounds of the present invention are potential inhibitors of ENPP-1. The structure of the molecules is represented in the Formula I.

The present invention discloses a compound of Formula I or a pharmaceutically acceptable salt, hydrate, solvate, tautomer, or isomer thereof.

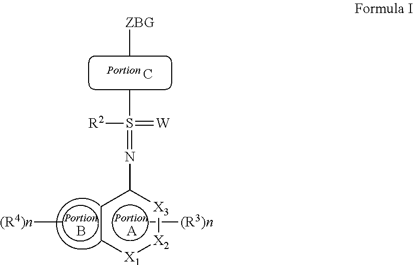

wherein the Formula I comprises portions A and B together constituting tail part, a portion C constituting core part, a sulfoximine-type group R2-S(═W)(═N) which links the tail part with the core part and a zinc binding group (ZBG), where,
the portion A is a six-membered aryl or heteroaryl, which is optionally substituted with one or more R³ groups and where X₁, X₂ and X₃ is CH, N, CR′ with the proviso that not more than two of X₁, X₂ and X₃ is simultaneously N;
the portion B is C6 aryl or 5-6 membered heteroaryl, optionally substituted with one or more R4 groups, wherein the portion B is fused to the portion A where the two shared atoms between the portions B and A come from a pair of carbon atoms or from a pair of atoms where one of the atoms is nitrogen and the other is carbon;
the portion C is selected from the group of structures consisting of (i), (ii), (iii), (iv), (v) and (vi),
where L1 is connected to (i) at variable positions of the ring
   where E1 is connected to the sulphur atom of the R2-S(═W)(═N) group and
   linker (L1) is connected to “a” atom of the zinc binding group (ZBG)

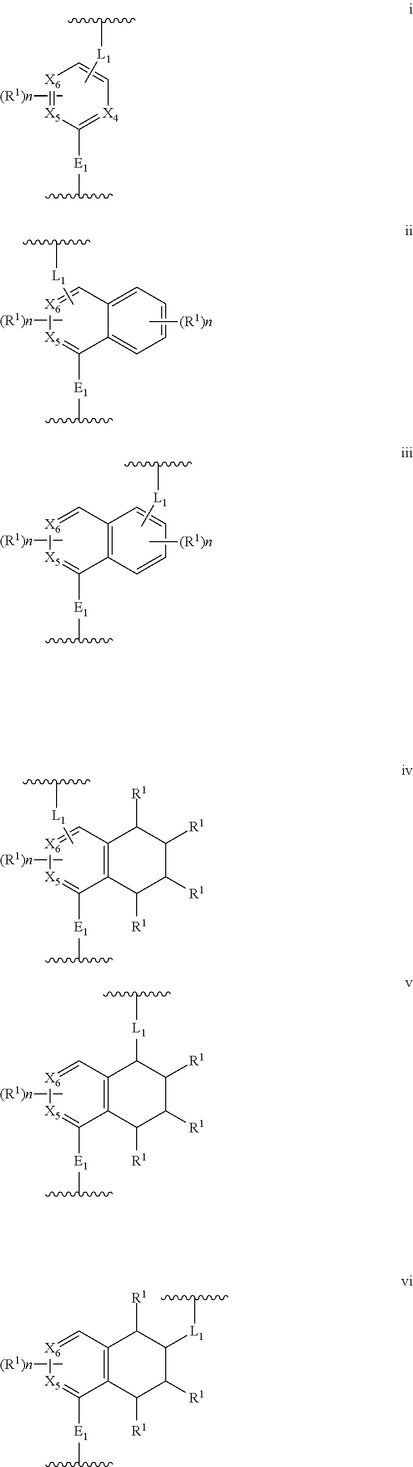

n=0,1,2,3,4 with the proviso that when n>1, the R¹ groups may be the same or different,
R¹, R³, R⁴ groups are selected from the group consisting of R′, halo, OR′, OAr, SR′, SAr, NHAr, NR′R′, CN, SCN, —NHCOR′, COR′, COOR′, COOAr, CF₃, CHF₂, CH₂F, OCF₃, SCF₃ and CH₂Ar, where R′═H, CN, C1-6 straight chain alkyl, branched chain alkyl, cycloalkyl, CH₂Ar and where Ar=aryl, substituted aryl, heteroaryl, or substituted hetero aryl;
E1=—(CH₂)_{y}— where y=0, 1 or 2;
L1=—(CH₂)_{y1}, NR″, O or S, C₁₋₆ straight chain alkyl, branched chain alkyl, —(CH₂)_{y1} cycloalkyl, where y1=0,1, 2, or 3;
Wherein R″═H, C1-6 straight chain alkyl, branched chain alkyl, cycloalkyl, aryl, substituted aryl, halogenated alkyl;
X₄, X₅ and X₆═CH, N or CR′;
the portion A is connected to nitrogen atom of the R2-S(═W)(═N) group
   where S is chiral sulphur atom,
   N is nitrogen,
   W is O or NH;
   R2 is selected from the group consisting of C1-6 straight, branched, cycloalkyl, alkenyl, alkylene, alkynyl, halo, aryl, heteroaryl, heterocycle, substituted aryl, CF₃, CHF₂, CH₂F, CN and a 2 to 6 membered alkylene group, one end of which is bonded to the sulphur atom and the other end bonded to portion C at the position alpha to the carbon atom of portion C which is bonded to sulphur atom when y=0, forming a cyclic structure and
ZBG as shown in structure (vii)

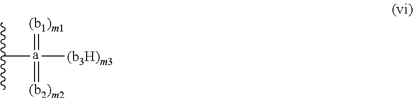

where a=S, P, C, or B atom;
b1, b2=O, S, NH or CH₂,
b3=O, S, NH, CH₂ or NH₂O
and m₁, m₂ and m₃=0, 1 with the proviso that only one of m₁, m₂ and m₃ can be 0 at a given instance

### Zinc Binding Domain (ZBG)

The zinc binding part (ZBG) is bonded to the core part through the linker L1, which may or may not be present.

In one embodiment, the zinc-binding part is formed from a group of structures consisting of

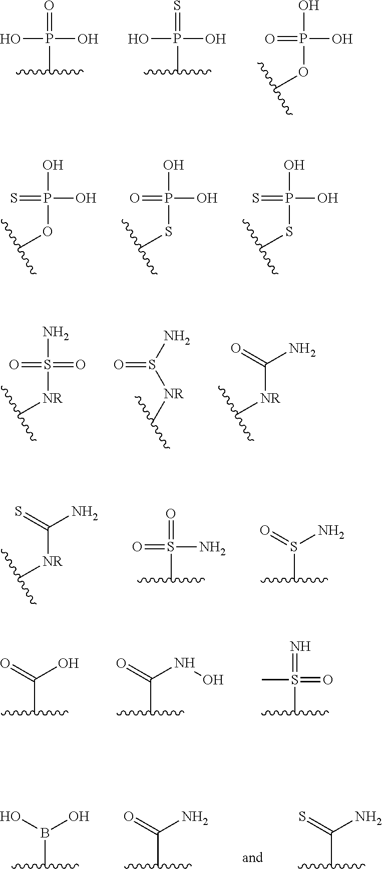

### Sulfoximine-Type Fragment

One of the important features of the structure depicted by Formula I is the presence of a sulfoximine-type fragment positioned between the tail and core parts. Sulfoximine based structures are steadily gaining popularity in medicinal science. One of the recent references is “Application of sulfoximines in medicinal chemistry from 2013 to 2020”, European Journal of Medicinal Chemistry 209, 112885 (2021). In another journal publication titled, “Sulfoximines as Rising Stars in Modern Drug Discovery? Current Status and Perspective on an Emerging Functional Group in Medicinal Chemistry” J. Med. Chem. 63 (23), 14243-14275 (2020), the limitations of this functional group are also discussed. The sulfoximine-type fragments of interest in this disclosure are given immediately below as sulfoximines and sulfondiimines.

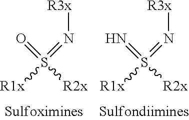

The sulfondiimines are isosteres of sulfoximines, and both fall under sulfoximine-type fragments in this disclosure. The nitrogen of NH group in sulfondiimine is bonded to sulphur through a double bond and is isosteric with 0 atom present in sulfoximine. In the present disclosure, the R1x has the same meaning as the R2 groups of the FIGURE I, R2x and R3x have the same meaning as core and tail parts of the FIGURE I respectively. None of the hitherto reported ENPP-1 inhibitors have the sulfoximine-type fragment positioned between core and tail parts of the inhibitor molecules. The sulfoximine-type fragment is represented as R2—S(═W)(═N) group in this disclosure.

In one embodiment, the R2 group of the sulfoximine-type fragment, is selected from the list of radicals consisting of methyl, cyclopropyl, cyclopropyl methyl, ethyl, propyl, isopropyl, phenyl, and benzyl.

The preferred sulfoximine-type fragments corresponding to the formula R2-S(═W)(═N), are

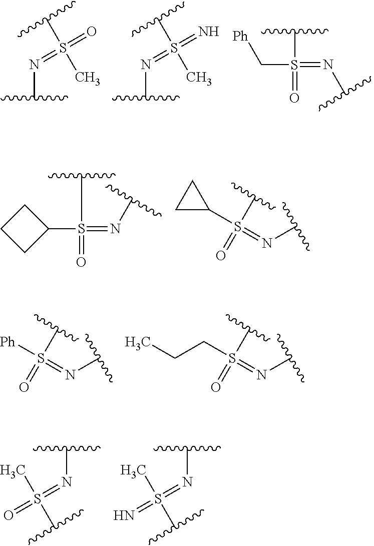

### The Tail Part

The tail part of FIGURE I is bonded to the nitrogen atom of the sulfoximine-type fragment, where the nitrogen is bonded to the sulphur atom through a double bond and the portion A is bonded to the nitrogen atom of the sulfoximine-type fragment.

In one embodiment, the tail part comprising portions A & B which contain X₁, X₂, X₃, R3 and R4 groups together is selected from the group consisting of

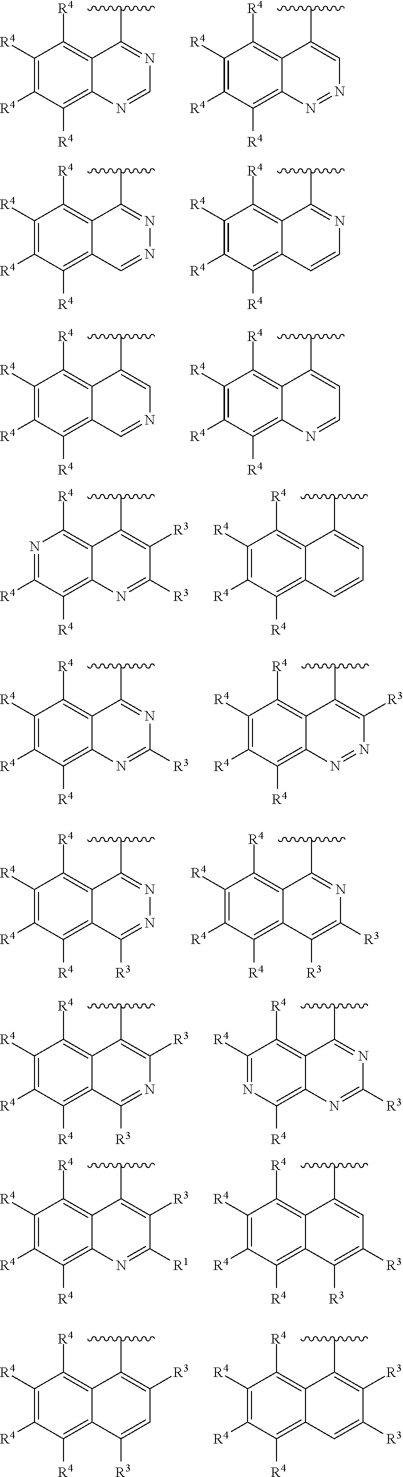

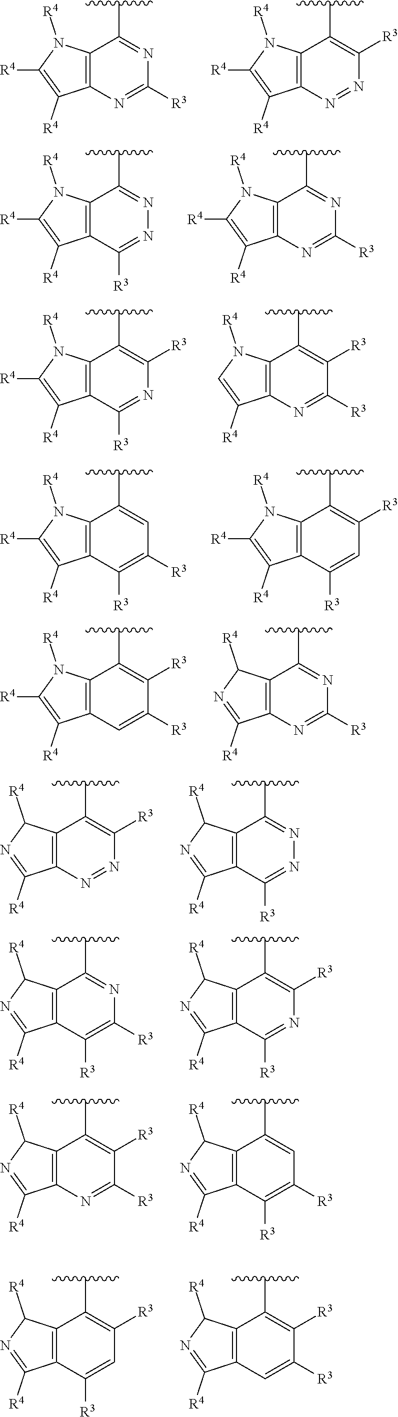

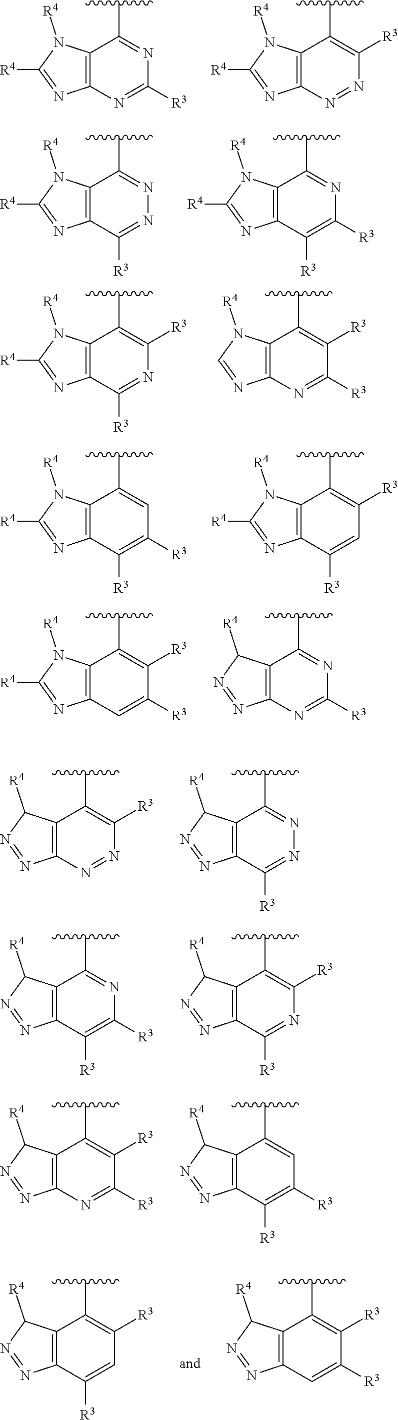

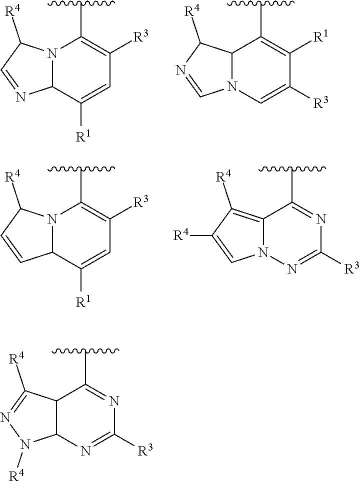

The number of ring substituents R3 and R4 on the portions A and B respectively may be 0, 1 or more. When more than one ring substituent is present, such ring substituents may be the same or different.

### The Core Part

The core part referred to as the portion C contains L1, X₄, X₅, X₆═, R1, L2 groups together and is selected from the group consisting of

[chemical structure]

[chemical structure]

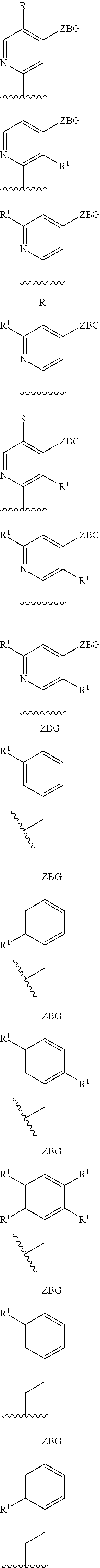

[chemical structure]

[chemical structure]

For most of these structures, one end of the core part is bonded to the ZBG and the other end of the core part shown by wavy lines is bonded to sulfoximine-type fragment, particularly the sulfur atom of the sulfoximine-type fragment.

In some of the structures of the core part as given below, the label, “Linker” is the sulfoximine-type fragment and the linkage of the core to the tail part through the nitrogen atom bonded to the sulphur is not shown in the structures immediately given below.

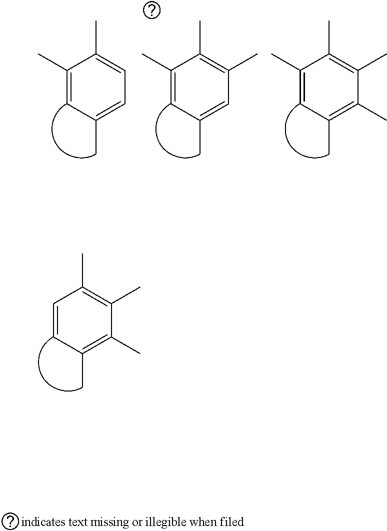

In one embodiment, the core part along with the sulfoximine-type fragment is selected from the group consisting of

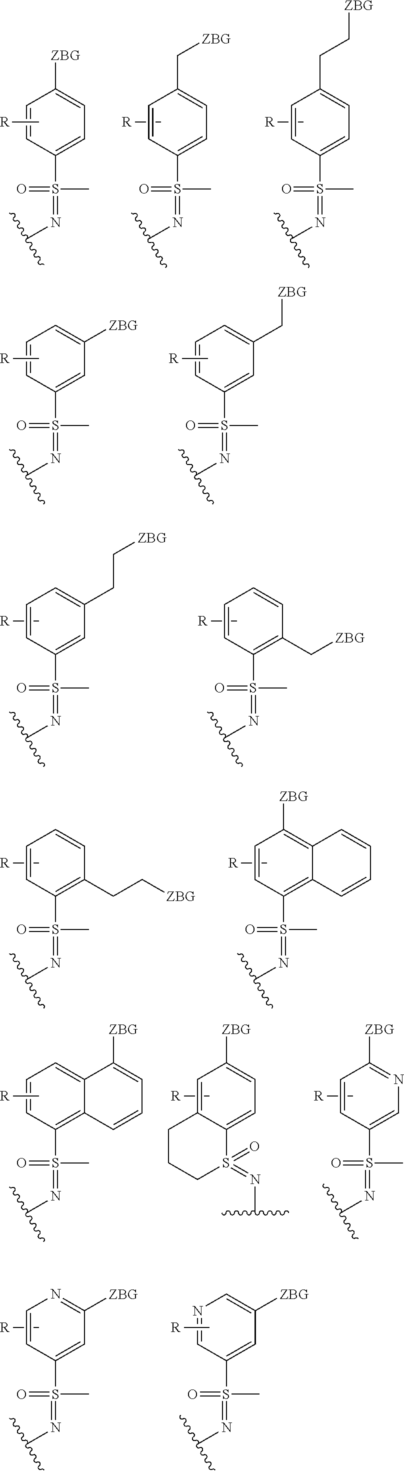

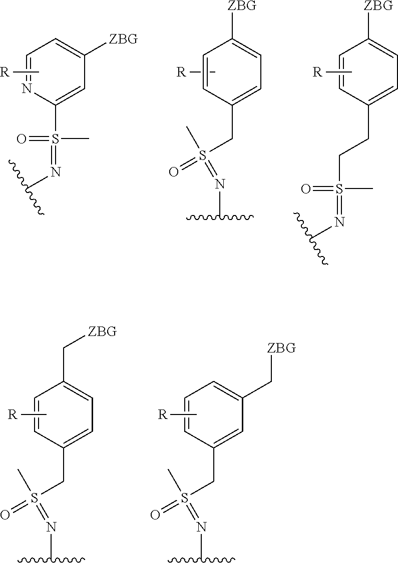

### Isomer of Formula

### Stereoisomers and Certain Constitutional Isomers

### Stereoisomers

The compounds corresponding to Formula I exhibit optical activity due to presence of asymmetric sulphur atom resulting in two non-superimposable mirror-image forms. The two forms of such compounds are known as enantiomers and classified as levorotary (1-isomer) or dextrorotary (d-isomer) depending on the rotation of plane-polarized light in a left (−) or right (+)-handed manner, respectively. Stereoisomer of FIGURE I is selected from a group consisting of (R) isomer of the formula I, (S) isomer of the Formula I and a combination thereof.

When both isomers have different activity or when only one of the isomer is showing activity and other is not showing any activity or when one is showing positive and other is showing negative activity, the chiral separation of such racemic drugs in pharmaceutical industries is important in order to remove unwanted isomer from composition and to achieve better therapeutic activity. If racemic is showing same activity as individual isomers, then no separation is required. The possible enantiomers of the invention are illustrated through an example below.

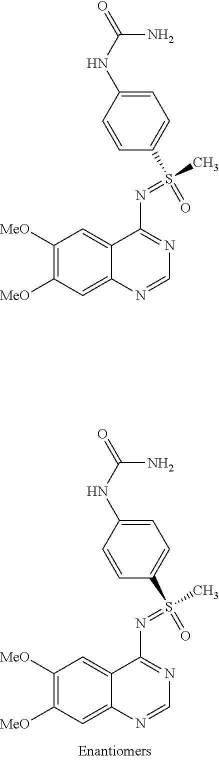 [SMILES: COc1cc2ncnc(N=[S@@](C)(=O)c3ccc(NC(N)=O)cc3)c2cc1OC ; COc1cc2ncnc(N=[S@](C)(=O)c3ccc(NC(N)=O)cc3)c2cc1OC]

### Constitutional Isomers (Also Called Positional Isomers)

The presence of N-substituted sulfoximine linker also provides an opportunity to generate positional or constitutional isomers. One of the positional isomers arises from variation of linkage of sulfoximine-type fragment of the Formula I to the core and tail parts. These structures vary from each other due to orientation of sufoximine linker. For illustration of constitutional isomers of Formula I, structure P1 presented below shows the attachment of N-substituted sufoximine linker to the tail part via Nitrogen atom of the sulfoximine linker and to the core part via S atom of the sulfoximine linker. Unlike the structure P1, the structure P2 represents the attachment of N-substituted sufoximine linker to the core part via N atom of the sulfoximine linker and to the tail part via S atom of the sulfoximine linker.

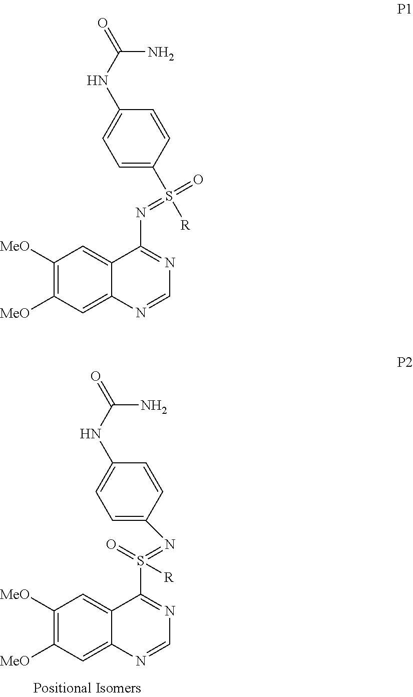

### Synthesis of Compounds of Formula I

### General Scheme of Synthesis of Inventive Examples of the Invention Represented by Formula I

The retrosynthetic strategy for the preparation of compounds represented by Formula I is to prepare aryl sulfoximine starting from a suitably substituted aryl alkyl sulfide through oxidation of the sulfide group using phenyl iodoacetate in the presence of ammonium carbamate. The preferred alkyl substituent is methyl.

All sulfoximine derivatives were synthesized from corresponding sulfides in presence of ammonia source and mild oxidizing reagents.

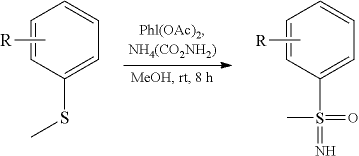

R groups on the sulfides are found in the derived sulfoximine molecules enumerated immediately below.

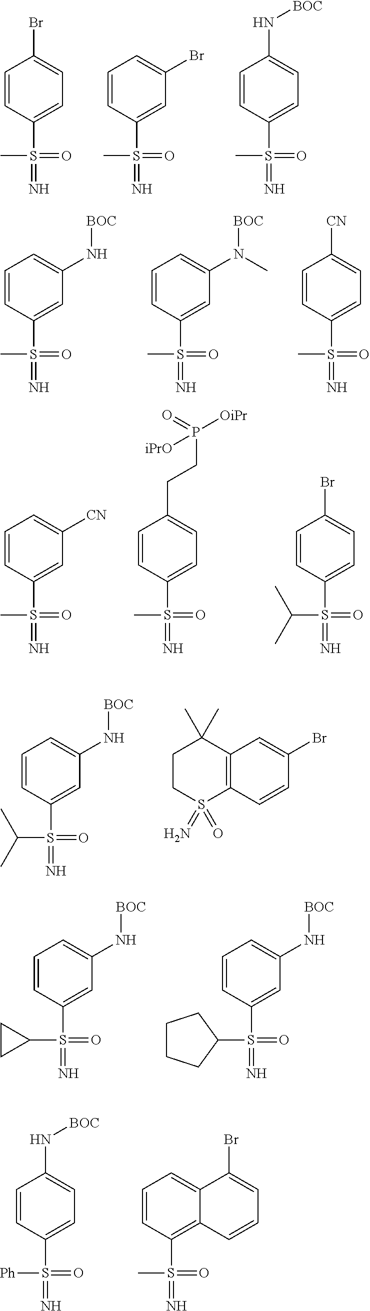

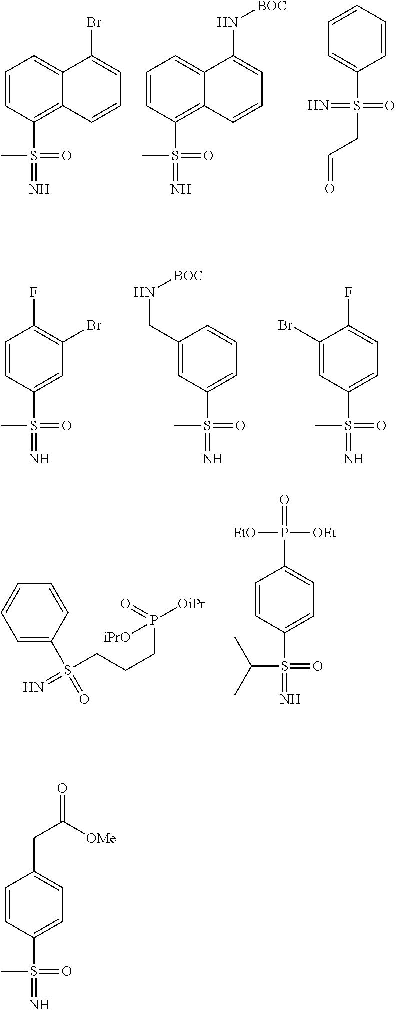

The aryl methyl sulfoximine molecule is then coupled to a halo compound in the presence of base to form the tail-sulfoximine-core structure which is then functionalized to have a zinc binding polar group. The products of the present invention are generally combination of (R) and (S) enantiomers or racemates and optionally resolved into the enantiomers.

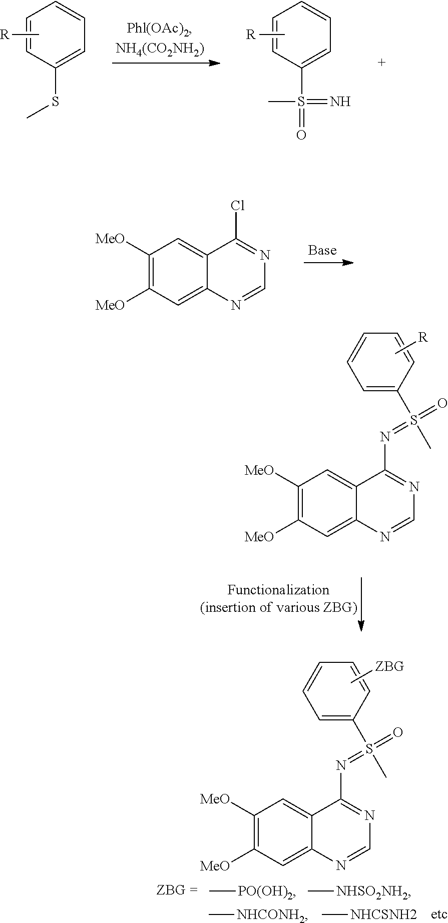

General Procedure: Substituted thioanisoles were treated with diacetoxyiodobenzene (PhI(OAc)2 and ammonium carbamate (NH4(CO2NH2) for the synthesis of sulfoximine. The corresponding sulfoximines were treated with base followed by 4-chloro-6,7-dimethoxyquinazoline to get respective sulfoximine-quinazoline coupled product. In the last step various zinc binding groups (ZBG) were inserted on phenyl ring attached with sulfoximine via functionalization as shown under Example 1.

### Example 1: Synthesis of {4-[N-(6,7-dimethoxyquinazoline-4-)-S-methanesulfonimidoyl]-phenyl}phosphonic acid (SAPTI012S001)

Step-1: 4-bromothioanisole (1.0 equivalent) was dissolved in MeOH (0.2M) in RBF, then Diacetoxyiodobenzene (PhI(OAc)2) (3.0 equivalent), followed by Ammonium carbamate (NH4(CO2NH2) (4.0 equivalent) were added portion wise under N2 atmosphere. After 2 hours again repeated the addition of same amount of (PhI(OAc)2) and (NH4(CO2NH2) to get maximum yield. After completion of reaction, methanol was removed under reduced pressure, and the reaction mixture dissolved in EtOAc and washed with water and brine solution. The organic layer was concentrated and purified with column chromatography by using 10-15% EtOAc in hexane as eluent. The product, 1-bromo-4-(S-methanesulfonimidoyl)benzene (SAPTI012S001/IM1) was confirmed with LCMS; yield-95%.

LCMS: [M+H]=234, 236 (isotopic peak).

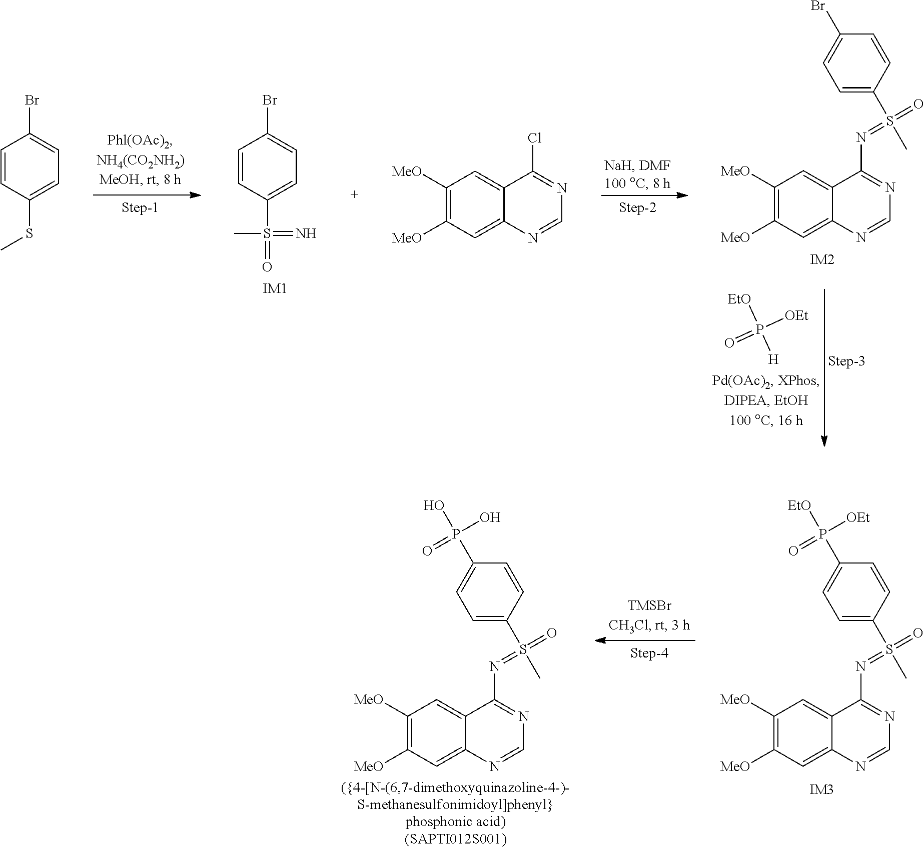

Step-2: 1-bromo-4-(S-methanesulfonimidoyl)benzene (1.0 equivalent) was dissolved in DMF (0.2M) in RBF and cooled to 0 °C with ice bath. Then NaH (2.0 equivalent) followed by 4-chloro-6,7-dimethoxyquinazoline (1.1 equivalent) were added under N2 atmosphere. Temperature of reaction mixture slowly was raised to 100 °C and stirred over 8 hr. After completion of reaction, mixture was quenched with water and extracted with EtOAc. The organic layer was concentrated and purified with column chromatography by using 30% of EtOAc in hexane as eluent. The product 4-{[(4-bromophenyl)(methyl)oxo-λ6-sulfanylidene]amino}-6,7-dimethoxyquinazoline (SAPTI012S001/IM2) confirmed with LCMS; yield-40%.

LCMS: [M+H]=422, 424 (isotopic peak).

Step-3: 4-{[(4-bromophenyl)(methyl)oxo-A6-sulfanylidene]amino}-6,7-dimethoxyquinazoline (1.0 equivalent), diethylphosphite (2.0 equivalent), DIPEA (1.5 equivalent), Pd(OAc)2 (5 mol %) and XPhos (10 mol %) were transferred to seal tube and dissolved with ethanol (0.2M). The reaction mixture was de-gassed under N2 atmosphere and stirred at 80 °C over 16 hr. After completion of reaction, the reaction mixture was quenched with water and extracted with ethyl acetate. The organic layer was concentrated and purified by column chromatography using 3-4% of DCM in hexane as eluent. The product diethyl(4-(N-(6,7-dimethoxyquinazolin-4-yl)-S-methylsulfonimidoyl)phenyl)phosphonate (SAPTI012S001/IM3) was confirmed with LCMS; yield-80%

LCMS: [M+H]=480

Step-4: diethyl(4-(N-(6,7-dimethoxyquinazolin-4-yl)-S-methylsulfonimidoyl)phenyl)phosphonate (1.0 equivalent) was dissolved in 0.2M dry CHCl3 in RBF followed by added TMSBr (4.0 equivalent) at 0 °C over 10 min and the reaction mass stirred at room temperature over 4 hr. After the completion of the reaction, diluted with methanol and concentrated under reduced pressure followed by recrystallized with solvent mixture of MeOH, DCM, and acetone. The pure product {4-[N-(6,7-dimethoxyquinazoline-4-)—S methanesulfonimidoyl]phenyl}phosphonic acid (SAPTI012S001) was confirmed with HNMR and LCMS; yield-50%, Purity-92%

LCMS: [M+H]=424.

¹H-NMR: 8.70 ppm (s, 1H); 8.16 ppm (d, 2H); 7.95 ppm (d, 2H); 7.67 ppm (s, 1H); 7.21 ppm (s, 1H); 3.99 ppm (s, 6H, OCH3); 3.85 ppm (s, 3H, SCH3).

### Example 2: Synthesis of {3-[N-(6,7-dimethoxyquinazoline-4-)-S-methanesulfonimidoyl]-phenyl}phosphonic acid (SAPTI012S002)

Step-1: 3-bromothioanisole (1.0 equivalent) was dissolved in MeOH (0.2M) in RBF, then Diacetoxyiodobenzene (PhI(OAc)2) (3.0 equivalent), followed by Ammonium carbamate (NH4(CO2NH2) (4.0 equivalent) were added portion wise under N2 atmosphere. After 2 hours again repeated the addition of same amount of (PhI(OAc)2) and (NH4(CO2NH2) to get maximum yield. After completion of reaction, methanol was removed under reduced pressure, and the reaction mixture dissolved in EtOAc and washed with water and brine solution. The organic layer was concentrated and purified with column chromatography by using 10-15% of EtOAc in hexane as eluent. The product 1-bromo-3-(S-methanesulfonimidoyl)benzene (SAPTI012S002/IM1) was confirmed with LCMS; yield-85%. LCMS: [M+]=234; 236 (isotopic peak)

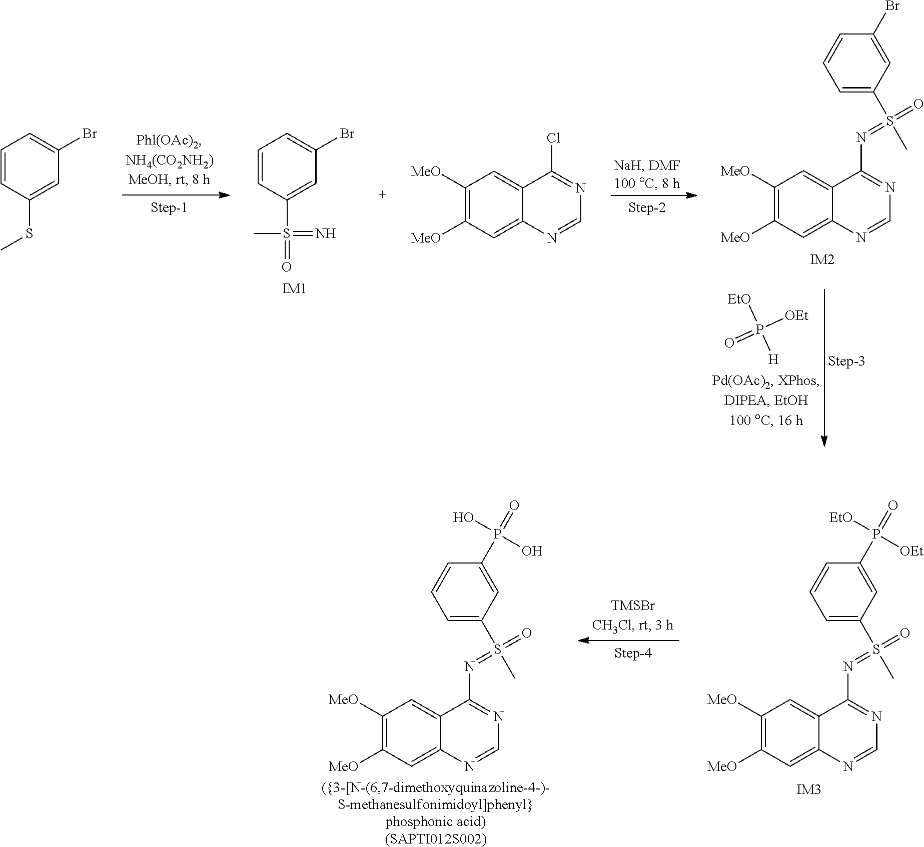

Step-2: 1-bromo-3-(S-methanesulfonimidoyl)benzene (1.0 equivalent) was dissolved in DMF (0.2M) in RBF and cooled to 0 °C with ice bath. Then NaH (2.0 equivalent) followed by 4-chloro-6,7-dimethoxyquinazoline (1.1 equivalent) were added under N2 atmosphere and temperature of reaction mixture slowly raised to 100 °C and stirred over 8 hr. After completion of reaction, mixture was quenched with water and extracted with EtOAc. The organic layer was concentrated and purified with column chromatography by using 30% of EtOAc in hexane as eluent. The product 4-{[(3-bromophenyl)(methyl)oxo-λ6-sulfanylidene]amino}-6,7-dimethoxyquinazoline (SAPTI012S002/IM2) confirmed with LCMS; yield-42%.

LCMS: [M+]=422; 424 (isotopic peak)

Step-3: 4-{[(3-bromophenyl)(methyl)oxo-A6-sulfanylidene]amino}-6,7-dimethoxyquinazoline (1.0 equivalent), diethylphosphite (2.0 equivalent), DIPEA (1.5 equivalent), Pd(OAc)2 (5 mol %) and XPhos (10 mol %) were transferred to seal tube and dissolved with ethanol (0.2M). The reaction mixture was de-gassed under N2 atmosphere and stirred at 80 °C over 16 hr. After completion of reaction, the reaction mixture was quenched with water and extracted with ethylacetate. The organic layer was concentrated and purified by column chromatography using 3-4% of DCM in hexane as eluent. The product diethyl(3-(N-(6,7-dimethoxyquinazolin-4-yl)-S-methylsulfonimidoyl)phenyl)phosphonate (SAPTI012S002/IM3) was confirmed with LCMS; yield-80%

LCMS: [M+H]=479.

Step-4: diethyl(3-(N-(6,7-dimethoxyquinazolin-4-yl)-S-methylsulfonimidoyl)phenyl)phosphonate (1.0 equivalent) was dissolved in 0.2M dry CHCl3 in RBF followed by added TMSBr (4.0 equivalent) at 0 °C over 10 min and the reaction mass stirred at room temperature over 4 hr. After the completion of the reaction, diluted with methanol and concentrated under reduced pressure followed by recrystallization with solvent mixture of MeOH, DCM, and acetone. The pure product {3-[N-(6,7-dimethoxyquinazoline-4-)-S-methanesulfonimidoyl]phenyl}phosphonic acid (SAPTI012S002) was confirmed with ¹H-NMR and LCMS; yield-44%, Purity-97%

LCMS: [M+H]=424.

¹H-NMR: 8.80 ppm (s, 1H, CH); 8.35 ppm (d, 1H, CH); 8.25 ppm (d, 1H, CH); 8.04 ppm (dd, 1H, CH); 7.82 ppm (m, 1H, CH); 7.69 ppm (s, 1H, CH); 7.24 ppm (s, 1H, CH); 4.01 ppm (s, 3H, OCH3); 4.00 ppm (s, 3H, OCH3); 3.92 ppm (s, 3H, SCH3).

### Example 3: Synthesis of Common Intermediate (SAPTI012S003/IM4) 4-[N-(6,7-Dimethoxy quinazoline-4-)-S-methanesulfonimidoyl]aniline

Step-1: 4-(methylsulfanyl)aniline (1.0 equivalent) in DCM solution was treated with N-ethyl, N,N-diisopropylamine (2.0 equivalent) at 0 °C followed by Boc-Anhydride (1.2 equivalent) was added at same temperature as dropwise. Temperature was raised slowly to room temperature and reaction mixture was stirred over 12 hr at RT. After completion of 4-(methylsulfanyl)aniline, the reaction mass was further diluted with DCM and washed with water and brine solution. The organic layer was concentrated and purified with column chromatography by using 10-15% of EtOAc in hexane as eluent. The isolated spot was non-polar than starting material and used in further step without purifications; yield-81%.

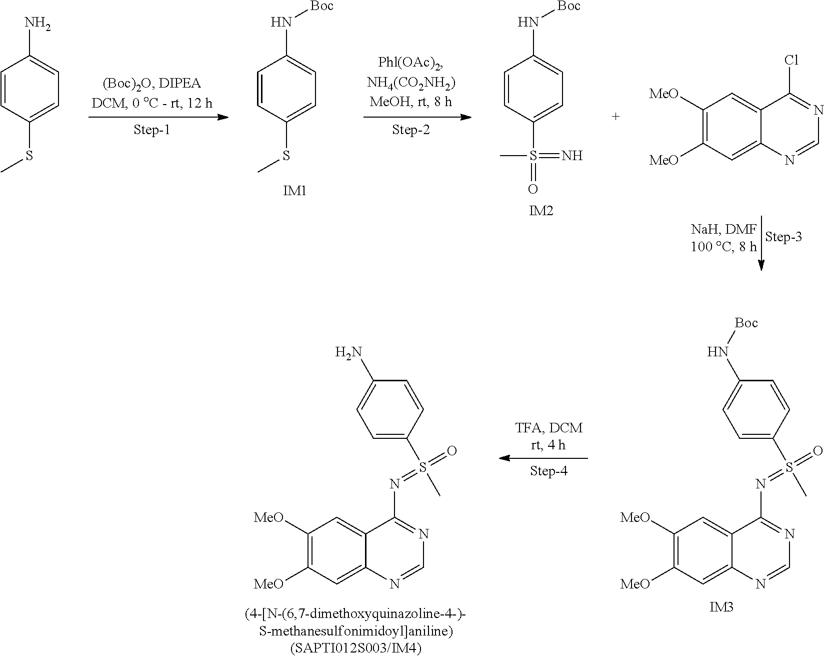

Step-2: tert-butyl [4-(methylsulfanyl)phenyl]carbamate (1.0 equivalent) was dissolved in MeOH (0.2M) in RBF, then Diacetoxyiodobenzene (PhI(OAc)2) (3.0 equivalent), followed by Ammonium carbamate (NH4(CO2NH2) (4.0 equivalent) were added portion wise under N2 atmosphere. After 2 hour again repeated the addition of same amount of (PhI(OAc)2) and (NH4(CO2NH2) to get maximum yield. After completion of reaction, methanol was removed under reduced pressure, and the reaction mixture dissolved in EtOAc and washed with water and brine solution. The organic layer was concentrated and purified with column chromatography by using 10-15% of EtOAc in hexane as eluent. The product tert-butyl[4-(S-methanesulfonimidoyl)phenyl]carbamate (SAPTI012S003/IM2) was confirmed with LCMS; yield 90%.

LCMS: [M+H]=271.

Step-3: tert-butyl[4-(S-methanesulfonimidoyl)phenyl]carbamate (1.0 equivalent) was dissolved in DMF (0.2M) in RBF and cooled to 0 °C with ice bath. Then NaH (2.0 equivalent) followed by 4-chloro-6,7-dimethoxyquinazoline (1.1 equivalent) were added under N2 atmosphere, and the temperature of reaction mixture slowly raised to 100 °C and stirred over 8 hr. After completion of reaction, mixture was quenched with water and extracted with EtOAc. The organic layer was concentrated and purified with column chromatography by using 30% of EtOAc in hexane as eluent. The product tert-butyl(3-(N-(6,7-dimethoxyquinazoline-4-yl)-S-methylsulfonimidoyl)phenyl)cait amate (SAPTI012S003/IM3) was confirmed with LCMS; yield-36%.

LCMS: [M+H]=459.

Step-4: tert-butyl(3-(N-(6,7-dimethoxyquinazoline-4-yl)-S-methylsulfonimidoyl)phenyl) carbamate (1.0 equivalent) was dissolved in 0.2M dry DCM in RBF followed by added trifluoroacetic acid (10.0 equivalent) at RT and stirred over 4 hr. The crude reaction mixture concentrated under reduced pressure to remove DCM and excess TFA. The TFA salt of free amine was neutralized with triethylamine to get free amine (4-[N-(6,7-dimethoxyquinazoline-4-)-S-methanesulfon imidoyl]aniline) (SAPTI012S003/IM4) and confirmed with LCMS of crude. The free amine in crude mixture was subjected to further reaction without purification.

LCMS: [M+H]=359.

### Example 4: Synthesis of N-{4-[N-(6,7-dimethoxyquinazoline-4-)-S-methanesulfonimidoyl]phenyl}sulfuric diamide (SAPTI012S003)

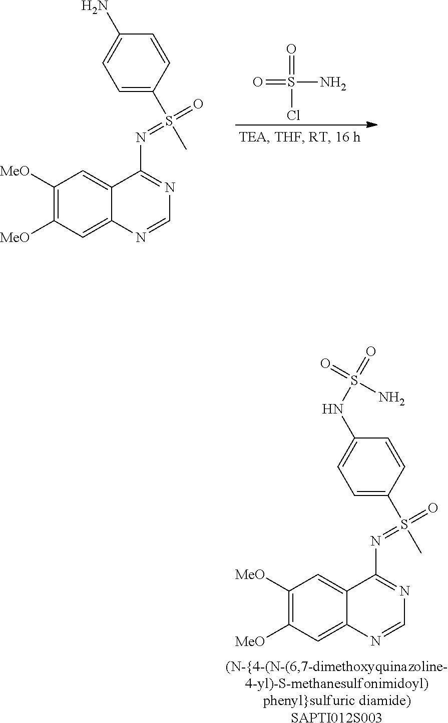

(4-[N-(6,7-dimethoxyquinazoline-4-)-S-methanesulfon imidoyl]aniline) (1.0 equivalent) was dissolved in dry THF (0.2M) in RBF, followed by added TEA (2.0 equivalent) and chlorosulfonamide (1.5 equivalent) under N2 atmosphere and the reaction mixture was stored over 16 hr at room temperature. After completion of reaction, THE was removed under reduced pressure and the crude was purified with column chromatography by using DMC & MeOH as eluent (3-4%). The product N-{4-[N-(6,7-dimethoxyquinazoline-4-)-S-methanesulfonimidoyl]phenyl}sulfuric diamide was confirmed with LCMS; yield-50%, Purity-90%.

LCMS: [M+H]=408.

¹H-NMR: 8.42 ppm (s, 1H, CH); 8.63 ppm (s, 1H, NH); 7.96 ppm (d, 2H, CH); 7.69 ppm (s, 1H, CH); 7.46 ppm (s, 2H, NH2); 7.34 ppm (d, 2H, CH); 7.21 ppm (s, 1H, CH); 3.94 ppm (s, 3H, OCH3); 3.90 ppm (s, 3H, OCH3); 3.69 ppm (s, 3H, SCH3).

### Example 5: Synthesis of 1-(3-(N-(6,7-dimethoxyquinazoline-4-yl)-S-methylsulfonimidoyl)-phenyl urea (SAPTI012S004)

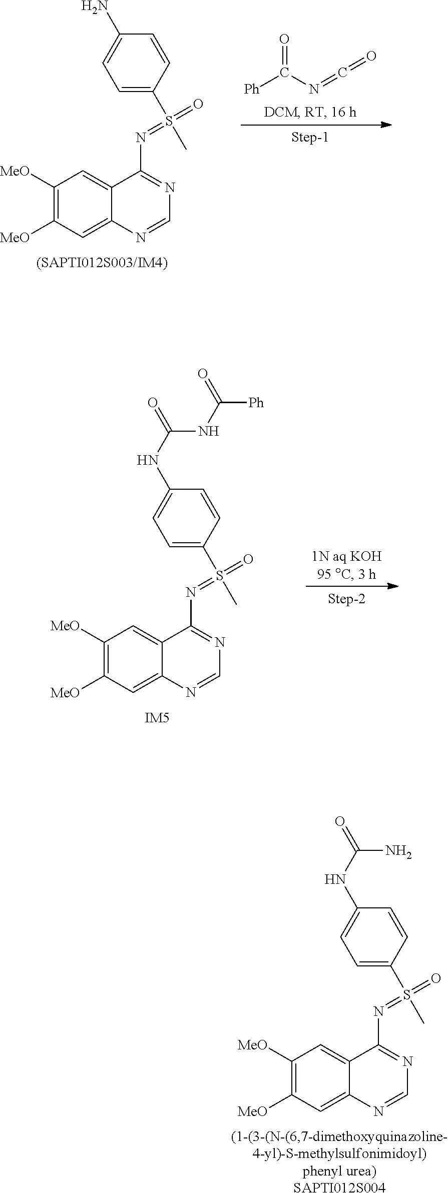

Step-1: (4-[N-(6,7-dimethoxyquinazoline-4-)-S-methanesulfonimidoyl]aniline) (1.0 equivalent) in DCM solution was treated with Benzoyl isocyanate (1.5 equivalent) at room temperature over 12 hr. After completion of reaction, the mass was further diluted with DCM and washed with water and brine solution. The organic layer concentrated under reduced pressure and purified with column chromatography by using 10-15% of DCM in MeOH as eluent. The product N-((3-(N-(6,7-dimethoxyquinazoline-4-yl)-S-methylsulfonimidoyl)phenyl)carbomoyl)benzamide (SAPTI012S004/IM5) was isolated with yield-33%.

Step-2: N-((3-(N-(6,7-dimethoxyquinazoline-4-yl)-S-methylsulfonimidoyl)phenyl) carbomoyl)benzamide (1.0 equivalent) was treated with 1.0 N aqueous KOH at 95 °C over 3 hr. Then the reaction mixture further diluted with water and extracted with DCM. The organic layer was concentrated and purified with column chromatography by using 3-4% of DCM in MeOH as eluent. The product 1-(3-(N-(6,7-dimethoxyquinazoline-4-yl)-S-methylsulfon imidoyl)phenyl urea (SAPTI012S004) was confirmed with LCMS; yield-66%, Purity-95%.

LCMS: [M+H]=402.

¹H-NMR: 9.72 ppm (s, 1H, NH); 8.36 ppm (s, 1H, CH); 7.89 ppm (d, 2H, CH); 7.67 ppm (d, 2H, CH); 7.57 ppm (s, 1H, CH); 7.15 ppm (s, 1H, CH); 6.27 ppm (s, 2H, NH2); 3.93 ppm (s, 3H, OCH3); 3.92 ppm (s, 3H, OCH3); 3.62 ppm (s, 3H, SCH3).

### Example 6: Synthetic Procedure for the Synthesis of SAPTI012S005

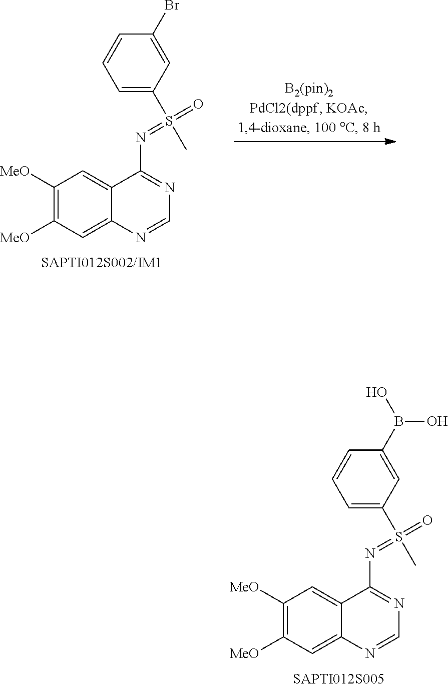 [SMILES: COc1cc2ncnc(N=S(C)(=O)c3cccc(Br)c3)c2cc1OC ; "SAPTI012S005" ; COc1cc2ncnc(N=S(C)(=O)c3cccc(B(O)O)c3)c2cc1OC]

The previous intermediate SAPTI012S002/IM1 (1.0 eq.) was dissolved in dry 1,4-dioxane (0.2M), then B2(Pin)2 (1.4 eq.) KOAc (3.5 eq.), followed by PdCl2(dppf) complex (5 mol %) were added and the reaction mixture was purged with N2 for 10 mins, later slowly raise the temperature to 100 °C, and stirred at same temperature for 8 hours. After completion of reaction, the reaction mixture was filtered through a pad of celite, and filtrate was evaporated and purified with silica gel column chromatography by using DCM & MeOH as eluent (0-5%). The product SAPTI012S005 was confirmed with LCMS and 1H NMR; yield 40%.

LCMS: [M+H]=388.

### Example 7: Synthetic Procedure for the Synthesis of SAPTI012S006

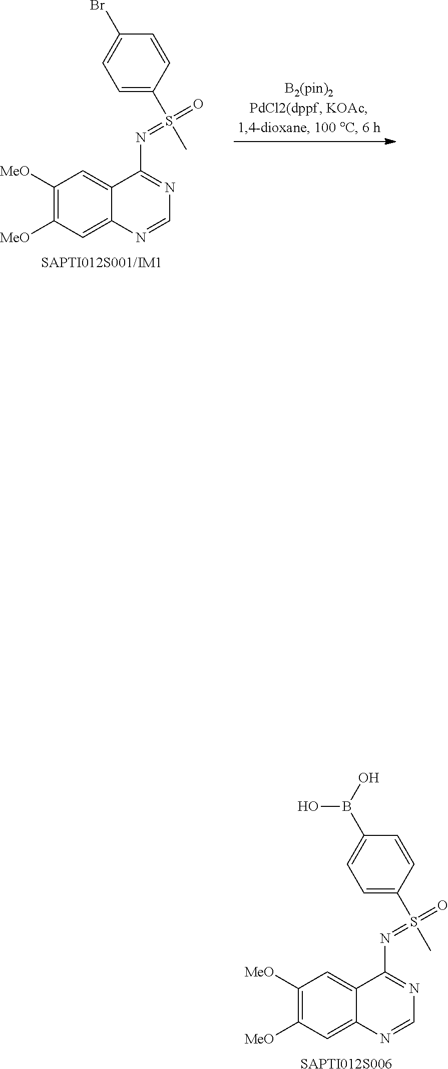 [SMILES: COc1cc2ncnc(N=S(C)(=O)c3ccc(Br)cc3)c2cc1OC ; "SAPTI012S006" ; COc1cc2ncnc(N=S(C)(=O)c3ccc(B(O)O)cc3)c2cc1OC]

The previous intermediate SAPTI012S001/IM1 (1.0 eq.) was dissolved in dry 1,4-dioxane (0.2M), then KOAc (3.5 eq.), B2(Pin)2 (1.4 eq.) followed by PdCl2(dppf) complex (5 mol %) were added and the reaction mixture was purged with N2 for 10 mins, later slowly raise the temperature to 100 °C and stirred at same temperature for 6 hours. After completion of reaction, the reaction mixture was filtered through a pad of celite, and filtrate was evaporated and purified with silica gel column chromatography by using DCM & MeOH as eluent (0-5%). The product SAPTI012S002 was confirmed with LCMS and 1H NMR; yield 40%; LCMS: [M+H]=388.

### Example 8: Synthetic Procedure for the Synthesis of SAPTI012S007

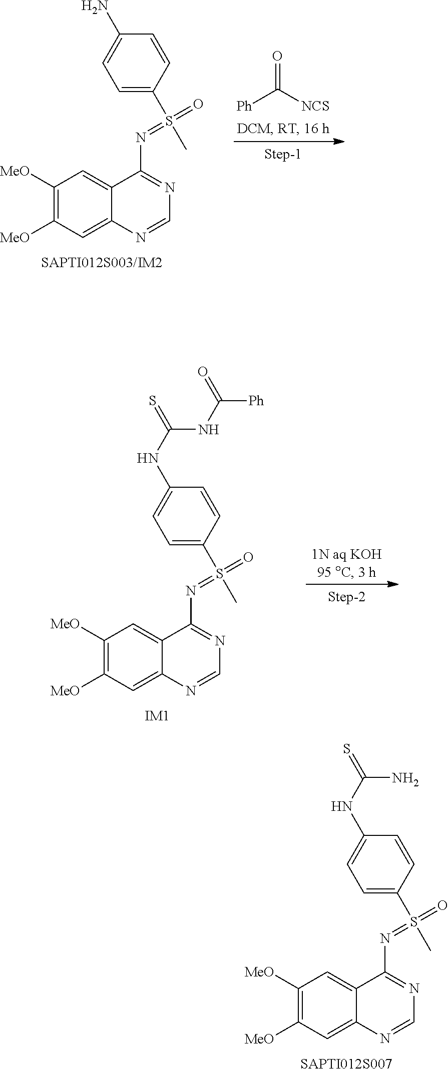

Step-1: The previous intermediate SAPTI012S003/IM2 (1.0 eq.) in DCM solution was treated with Bezoylthioisocyanate (1.5 eq.) at room temperature for 12 hours. After completion of reaction, the mass was further diluted with DCM and washed with water and brine solution. The combined organic layer was concentrated under reduced pressure and purified with column chromatography by using DCM & MeOH as eluent (10-15%). The product SAPTI012S007/IM1 was confirmed with LCMS; yield-33%.

LCMS: [M+1]=522.

Step-2: The intermediate SAPTI012S007/IM1 (1.0 eq.) was treated with 1.0 N aq KOH at 95 °C for 3 hours. Then the reaction mixture was further diluted with water and extracted with DCM. The organic layer was concentrated and purified with column chromatography by using DCM & MeOH as eluent (3-4%). The product SAPTI012S007 was confirmed with LCMS and 1H NMR; yield-66%.

LCMS: [M+H]=418.

### Example 9: Synthetic Procedure for the Synthesis of SAPTI012S008

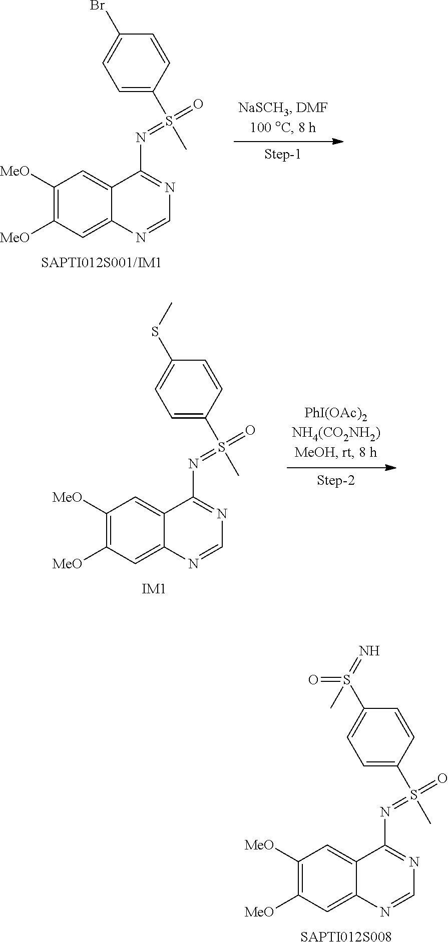

Step-1: The previous intermediate SAPTI012S001/IM1 (1.0 eq.) was dissolved in DMF (0.2M) followed by sodium thiomethoxide (1.6 eq.) was added at room temperature and the resulting mixture was stored at 100 °C for 8 hours. After the reaction completion, reaction mixture was quenched with MeOH, and evaporated the solvent under rotary evaporator till complete DMF removal. The reaction mixture again dissolved in sufficient DCM and washed with water and brine solution. The organic layer was concentrated to get crude reaction mixture and the crude was purified with silica gel column chromatography by using DCM & MeOH as eluent (0-5%). The product SAPTI012S008/IM1 was confirmed with LCMS; Yield: 70.6%; LCMS: [M+H]=390

Step-2: The intermediate SAPTI012S008/IM1 (1.0 eq.) was dissolved in MeOH (0.2M) followed by Diacetoxyiodobenzene (PhI(OAc)2) (1.5 eq.), and Ammonium carbamate (NH4(CO2NH2) (2.0 eq.) were added portion wise, and the reaction course was monitored with TLC and LCMS. If the starting material still remained even after 2 hours, again 0.5 eq. of PhI(OAc)2 and 0.6 eq. of NH4(CO2NH2) were added to get maximum yield. After completion of reaction, methanol was removed under reduced pressure, and the reaction mixture was dissolved in EtOAc and washed with water and brine solution. The combined organic layer was concentrated and purified with column chromatography by using EtOAc & Hexane as eluent (85-90%). The SAPTI012S008 was confirmed with LCMS, and 1H NMR; Yield: 30%.

LCMS: [M+H]=421

### Example 10: Synthetic Procedure for the Synthesis of SAPTI012S009

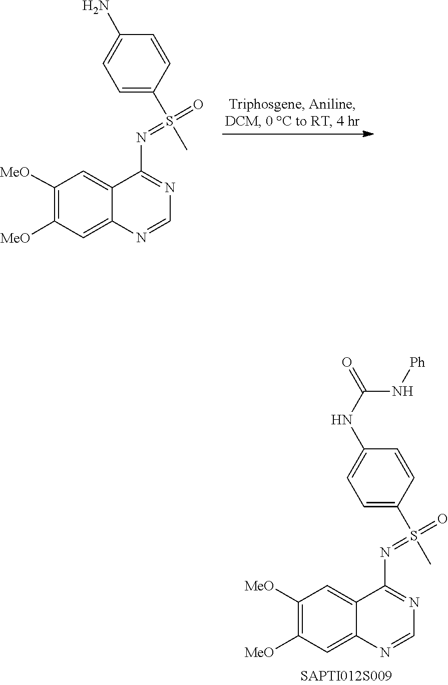 [SMILES: COc1cc2ncnc(N=S(C)(=O)c3ccc(N)cc3)c2cc1OC ; COc1cc2ncnc(N=S(C)(=O)c3ccc(NC(=O)Nc4ccccc4)cc3)c2cc1OC]

Triphosgene (1.1 eq.) was dissolved in DCM (0.2M) then the reaction mixture was cooled to 0 °C under nitrogen atmosphere followed by aniline (1.0 eq.) was added slowly to the reaction and stirring was continued at room temperature for about 2 hours. Then starting material SAPTI012S003/IM2 amine (1.0 eq.) was added at RT and stirred for about 2-3 hrs. The progress of the reaction was monitored by TLC, later the reaction mixture quenched with aq NaHCO₃ at 0 °C and extracted with DCM. The combined organic layer was evaporated under reduced pressure to get crude. The crude was purified by using column chromatography with EtOAc & Hexane as eluant. The product SAPTI012S009 was confirmed with LCMS, and 1H NMR; yield-67%.

LCMS: [M+H]=478.

### Example 11: Synthetic Procedure for the Synthesis of SAPTI012S010

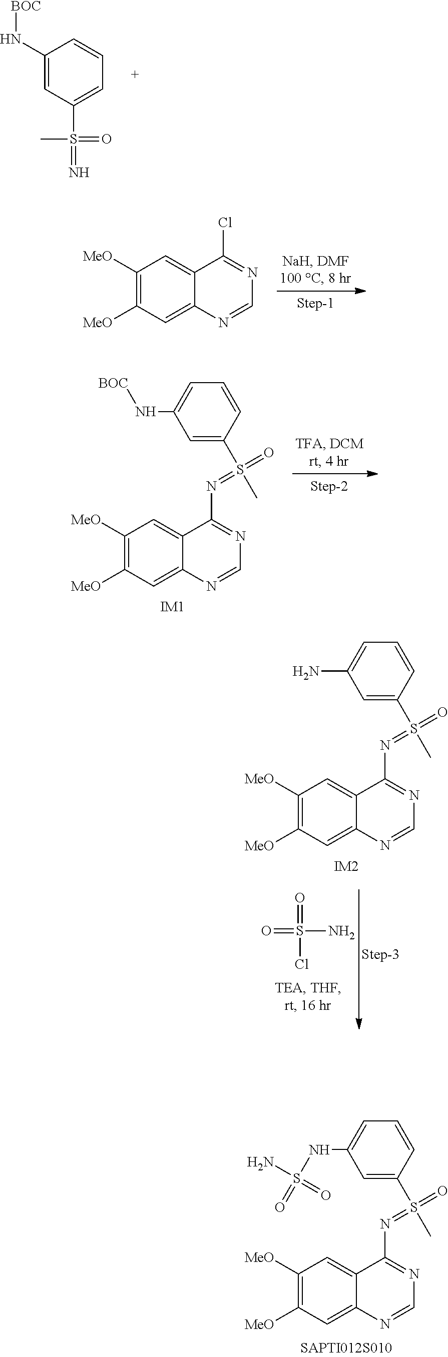

Step-1: tert-butyl[3-(S-methanesulfonimidoyl)phenyl]carbamate (1.0 eq.) was dissolved in DMF (0.2M) then cooled to 0 °C with ice bath. Then NaH (2.0 eq.) followed by 4-chloro-6,7-dimethoxyquinazoline (1.1 eq.) were added under N2 atmosphere, and the temperature of reaction mixture slowly was raised to 100 °C and stirred for 8 hours. After completion of the reaction, reaction mixture was quenched with water and extracted with EtOAc. The organic layer was concentrated and purified with column chromatography by using EtOAc & Hexane as eluent (60-70%). The product SAPTI012S010/IM1 was confirmed with LCMS; yield-36%.

LCMS: [M+H]=459.

Step-2: The intermediate SAPTI012S010/IM1 (1.0 eq.) was dissolved in 0.2M dry DCM in RBF followed by trifluoroacetic acid (10V) was added at RT and stirred for 4 hours. The crude reaction mixture concentrated under reduced pressure to remove DCM and excess TFA. Then TFA salt of free amine was neutralized with triethylamine to get free amine SAPTI012S010/IM2 and confirmed with LCMS of crude. The free amine in crude mixture was subjected to further reaction without further purification.

LCMS: [M+H]=359.

Step-3: The intermediate SAPTI012S010/IM2 (1.0 eq.) was dissolved in dry THF (0.2M) in RBF, followed by TEA (2.0 eq.) and chlorosulfonamide (1.5 eq.) was added under N2 atmosphere and the reaction mixture was stirred for 16 hours at room temperature. After completion of reaction, THF was removed under reduced pressure and the crude was purified with column chromatography by using DMC & MeOH as eluent (3-4%). The product SAPTI012S010 was confirmed with LCMS, and 1H NMR; yield-50%.

LCMS: [M+H]=438.

### Example 12: Synthetic Procedure for the Synthesis of SAPTI012S011

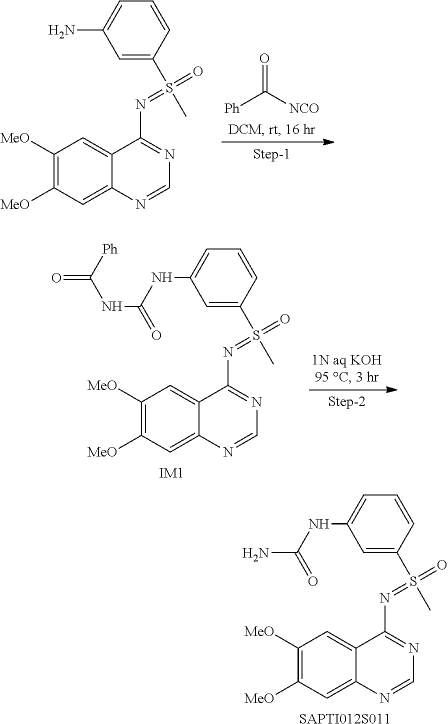

Step-1: The previous intermediate SAPTI012S010/IM2 (1.0 eq.) in DCM solution was treated with Benzyl isocyanate (1.5 eq.) at room temperature for 12 hours. After completion of reaction, the mass was further diluted with DCM and washed with water and brine solution. The combined organic layer was concentrated under reduced pressure and the crude was purified with column chromatography by using DCM & MeOH as eluent (10-15%). The product SAPTI012S011/IM1 was confirmed with LCMS; yield-57%.

LCMS: [M+H]=506Step-2: The intermediate SAPTI012S011/IM1 (1.0 eq.) was treated with 1.0 N aq KOH at 95 °C for 3 hours. Then the reaction mixture was further diluted with water and extracted with DCM. The organic layer was concentrated and purified with column chromatography by using DCM & MeOH as eluent (3-4%). The product SAPTI012S011 was confirmed with LCMS, 1H NMR; yield-67%.

LCMS: [M+H]=402.

### Example 13: Synthetic Procedure for the Synthesis of SAPTI012S012

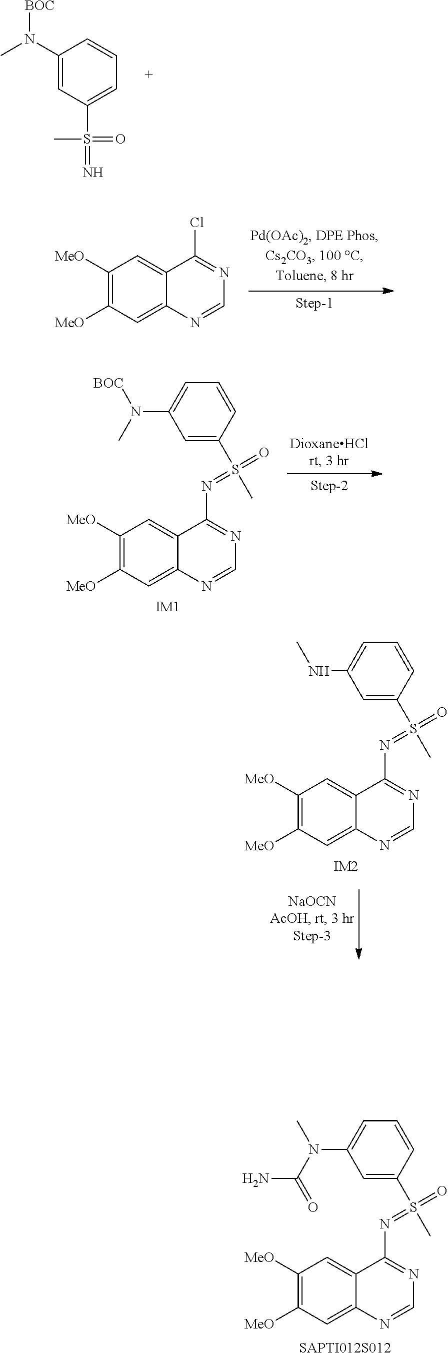

Step-1: t-butyl[3-(S-methanesulfonimidoyl)phenyl]methylcarbamate (1.0 eq.) was dissolved in toluene (0.2M) in RBF, then DPE Phos (7.5 mol %), Cs2CO3 (1.4 eq.) followed by 4-chloro-6,7-dimethoxyquinazoline (1.5 eq.) were added and the solution was purged with N2 for 10 mins, then Pd(OAc)2 (5 mol %) was added and slowly raise the temperature to 100 °C. The reaction mixture stirred under N2 atmosphere at 100 °C for 6 hours. After completion of reaction, the reaction mixture was filtered through a pad of celite, and filtrate was evaporated and purified with silica gel column chromatography by using DCM & MeOH as eluent (0-5%). The product SAPTI012S012/IM1 was confirmed with LCMS; yield 28%.

LCMS: [M+H]=473

Step-2: The intermediate SAPTI012S012/IM1 (1.0 eq.) was dissolved in 1,4-Dioxane (0.2M) in RBF and cooled to 0 °C with ice bath, then Dioxane HCl (10 vol.) was added dropwise and stirred for 5 hours at RT. After completion of reaction, the reaction mixture was evaporated to dryness, washed with ether. The HCl salt of SAPTI012S012/IM2 was confirmed with LCMS and used further without further purification; yield-64%.

LCMS: [M+H]=373

Step-3: The HCl salt of SAPTI012S012/IM2 (1.0 eq.) was dissolved in water and AcOH (1:1) followed by sodium cyanate (1 eq.) was added portion wise at 0 °C and the reaction was stirred at RT for 2 hours. After the completion of reaction, the reaction mixture was quenched with water and extracted with DCM multiple time, the combined organic layer was washed with water and brine solution and passed through anhydrous sodium sulphate and evaporated under reduced pressure and purified by reversed phase column chromatography by using water and MeOH as an eluent (20-30%). The product SAPTI012S012 was confirmed with LCMS and 1H NMR; yield: 30%

LCMS: [M+H]=416

### Example 14: Synthetic Procedure for the Synthesis of SAPTI012S013

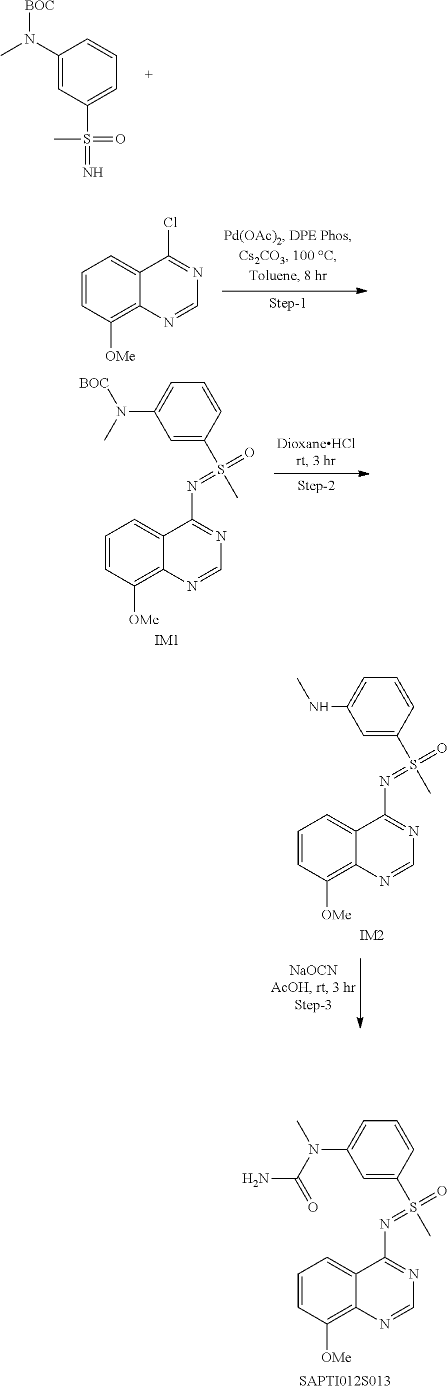

Step-1: t-butyl[3-(S-methanesulfonimidoyl)phenyl]methylcarbamate (1.0 eq.) was dissolved in toluene (0.2M) in RBF, then DPE Phos (7.5 mol %), Cs2CO3 (1.4 eq.) followed by 4-chloro-8-methoxyquinazoline (1.5 eq.) were added and the solution was purged with N2 for 10 mins, then Pd(OAc)2 (5 mol %) was added and slowly raise the temperature to 100 °C. The reaction mixture was stirred under N2 atmosphere at 100 °C for 6 hours. After completion of reaction, the reaction mixture was filtered through a pad of celite, and filtrate was evaporated and purified with silica gel column chromatography by using DCM & MeOH as eluent (0-5%). The product SAPTI012S013/IM1 was confirmed with LCMS; yield 64%.

LCMS: [M+H]=443

Step-2: The intermediate SAPTI012S013/IM1 (1.0 eq.) was dissolved in 1,4-Dioxane (0.2M) in RBF and cooled to 0 °C with ice bath. Then Dioxane HCl (10 vol.) was added dropwise and stirred for over 5 hours at RT. After completion of reaction, the reaction mixture was evaporated to dryness, washed with ether. The HCl salt of SAPTI012S013/IM2 was confirmed with LCMS and used further without further purification; yield-91%.

LCMS: [M+H]=343

Step-3: The HCl salt of SAPTI012S013/IM2 (1.0 eq.) was dissolved in water and AcOH (1:1) followed by sodium cyanate (1 eq.) was added portion wise at 0 °C and reaction was stirred at RT for 2 hours. After the completion of reaction, the reaction mixture was quenched with water and extracted with DCM multiple times, the combined organic layer was washed with brine solution and passed through anhydrous sodium sulphate and evaporated under reduced pressure and purified by reversed phase column chromatography by using water and MeOH as an eluent (20-30%). The product SAPTI012S013 was confirmed with LCMS and 1H NMR; yield: 20%.

LCMS: [M+H]=386

### Example 15: Synthetic Procedure for the Synthesis of SAPTI012S014

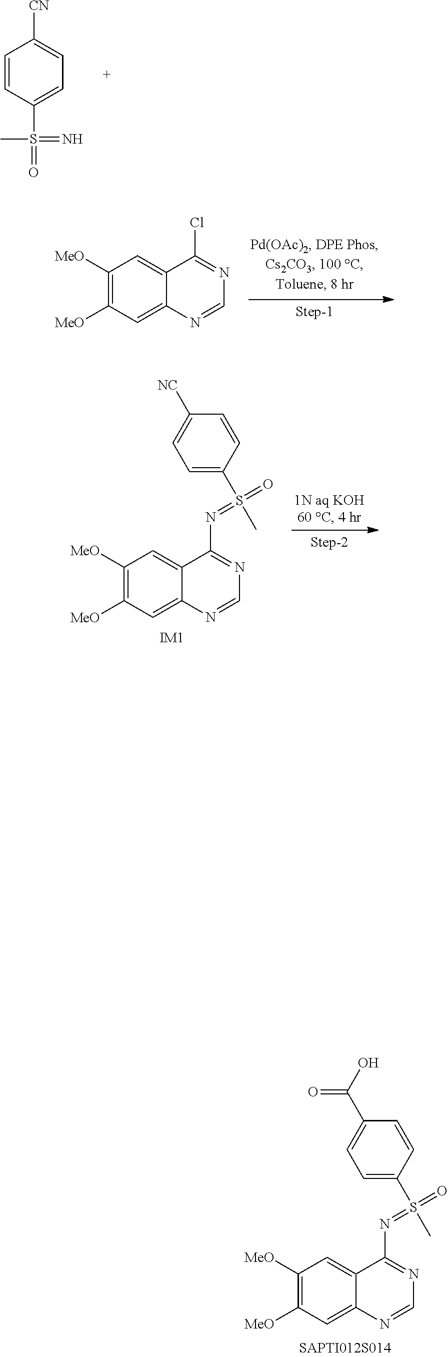

Step-1: 4-(S-methanesulfonimidoyl)benzonitrile (1.0 eq.) was dissolved in toluene (0.2M) in RBF, then DPE Phos (7.5 mol %) and Cs2CO3 (1.4 eq.) followed by 4-chloro-6,7-dimethoxyquinazoline (1.5 eq.) were added, and the solution purged with N2 for 10 mins. Then Pd(OAc)2 (5 mol %) was added and the temperature was raised to 100 °C. The reaction mixture stirred under N2 atmosphere at 100 °C for 6 hours. After completion of reaction, the reaction mixture was filtered through a pad of celite, and filtrate was evaporated and purified with silica gel column chromatography by using DCM & MeOH as eluent (0-5%). The product SAPTI012S014/IM1 was confirmed with LCMS; yield 27%.

LCMS: [M+H]=369

Step-2: SAPTI012S014/IM1 (1.0 eq.) was dissolved in 1 N KOH (10V) and resulting mixture was stirred at 60 °C for 4 hours. After completion of reaction, the reaction mixture diluted with water and extracted with DCM multiple times, and aqueous layer was acidified with 1 N HCl till aqueous layer become acidic solution was extracted with DCM and this organic layer collected and concentrated to get crude mixture. The crude was purified with silica gel column chromatography by using DCM & MeOH as eluent (0-10%). The product SAPTI012S014 was confirmed with LCMS and 1H NMR; Yield 42% LCMS [M+H]=388.

### Example 16: Synthetic Procedure for the Synthesis of SAPTI012S015

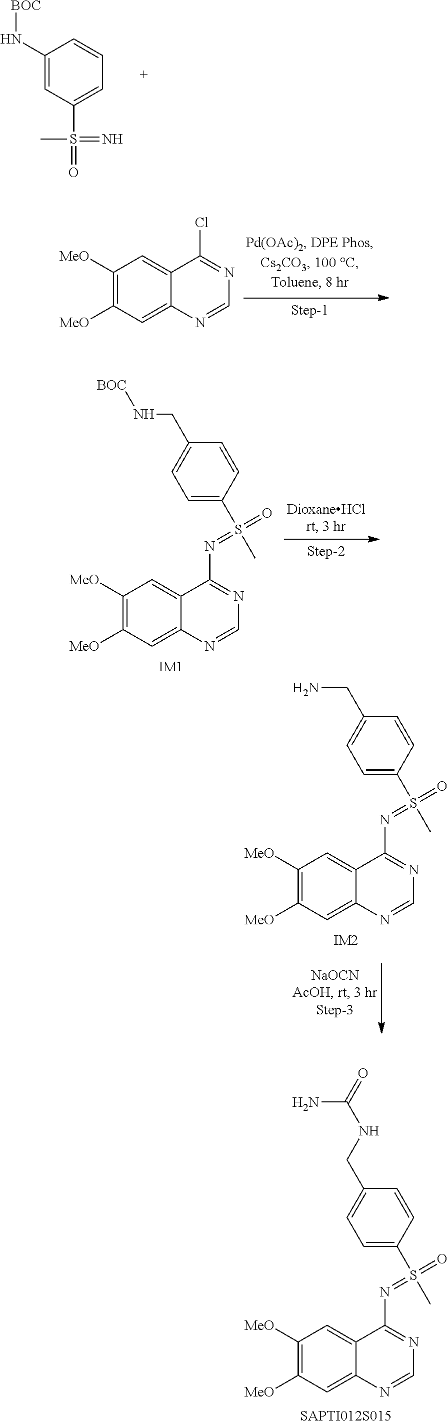

Step-1: tert-butyl {[3-(S-methanesulfonimidoyl)phenyl]methyl}carbamate (1.0 eq.) was dissolved in toluene (0.2M) in RBF, then DPE Phos (7.5 mol %), Cs2CO3 (1.4 eq.) followed by 4-chloro-6,7-dimethoxyquinazoline (1.5 eq.) were added and the solution was purged with N2 for 10 mins, then Pd(OAc)2 (5 mol %) was added and slowly raise the temperature to 100 °C. The reaction mixture stored under N2 atmosphere at 100 °C for 6 hours. After completion of reaction, the reaction mixture was filtered through a pad of celite, and filtrate was evaporated and purified with silica gel column chromatography by using DCM & MeOH as eluent (0-5%). The product SAPTI012S015/IM1 was confirmed with LCMS; yield 47%.

LCMS: [M+H]=473

Step-2: The intermediate SAPTI012S015/IM1 (1.0 eq.) was dissolved in 1,4-Dioxane (0.2M) in RBF and cooled to 0 °C with ice bath. Then Dioxane HCl (10 vol.) was added dropwise and stirred for 5 hours at RT. After completion of reaction, the reaction mixture was evaporated to dryness, washed with ether. The HCl salt of SAPTI012S015/IM2 was confirmed with LCMS and used further without further purification; yield-98%.

LCMS: [M+H]=373

Step-3: The SAPTI012S015/IM2 (1.0 eq.) was dissolved in water and AcOH (1:1) followed by sodium cyanate (1 eq.) was added portion wise at 0 °C and reaction was stirred at RT for 2 hours. After the completion of reaction, the reaction mixture was quenched with water and extracted with DCM multiple time, the combined organic layer was washed with brine solution and passed through anhydrous sodium sulphate and evaporated under reduced pressure and purified by reversed phase column chromatography by using water and MeOH as an eluent (20-30%). The product SAPTI012S015 was confirmed with LCMS and 1H NMR; yield: 32%

LCMS: [M+H]=416

### Example 17: Synthetic Procedure for the Synthesis of SAPTI012S016

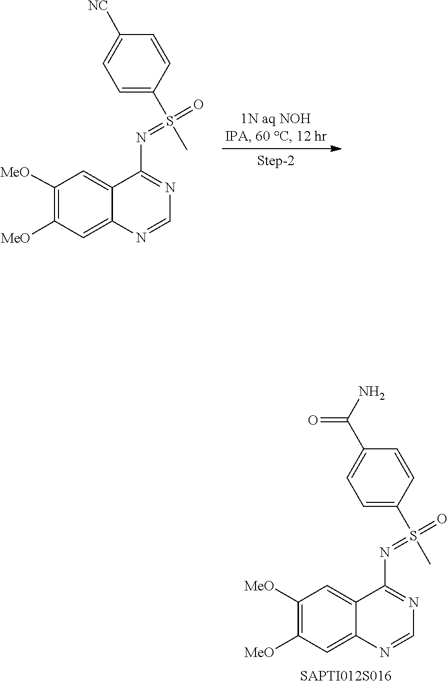 [SMILES: COc1cc2ncnc(N=S(C)(=O)c3ccc(C#N)cc3)c2cc1OC ; COc1cc2ncnc(N=S(C)(=O)c3ccc(C(N)=O)cc3)c2cc1OC]

The previous intermediate SAPTI012S014/IM1 (1.0 eq.) was dissolved in Isopropyl alcohol (10 v) followed by NaOH (1 eq.) was added and resulting mixture was stirred at 80 °C for 12 hours. After completion of reaction, the reaction mixture was evaporated to remove isopropyl alcohol from the reaction mixture. Resulting crude was diluted with water and extracted with DCM multiple times, the combined organic layer was concentrated to get crude mixture. The product was purified with silica gel column chromatography by using DCM & MeOH as eluent (0-10%). The product SAPTI012S016 was confirmed with LCMS and 1H NMR; Yield 50%

LCMS [M+H]=387

### Example 1: Synthetic Procedure Tor the Synthesis of SAPTI012S017

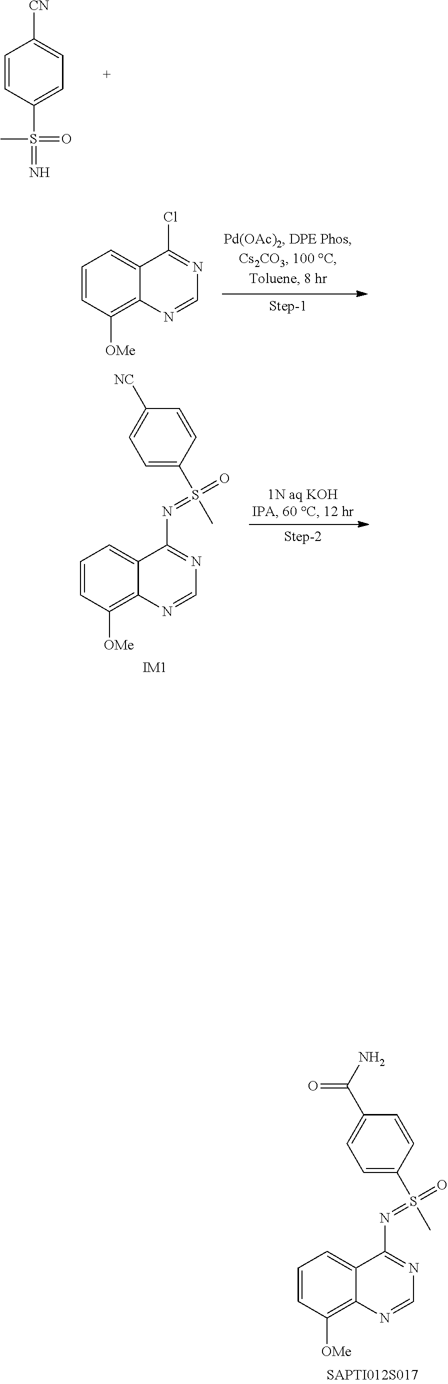

Step-1: 4-(S-methanesulfonimidoyl)benzonitrile (1.0 eq.) was dissolved in toluene (0.2M) in RBF, then DPE Phos (7.5 mol %), Cs2CO3 (1.4 eq.) followed by 4-chloro-8-methoxyquinazoline (1.5 eq.) were added and the solution was purged with N2 for 10 mins, then Pd(OAc)2 (5 mol %) was added and slowly raise the temperature to 100 °C. The reaction mixture stored under N2 atmosphere at 100 °C for 8 hours. After completion of reaction, the reaction mixture was filtered through a pad of celite, and filtrate was evaporated and purified with silica gel column chromatography by using DCM & MeOH as eluent (0-5%). The product SAPTI012S017/IM1 was confirmed with LCMS; yield 78%.

LCMS: [M+H]=339

Step-2: The intermediate SAPTI012S017/IM1 (1.0 eq.) dissolved in isopropyl alcohol (10 v) followed by NaOH (1 eq.) was added and resulting mixture was stirred at 80 °C for 12 hours. After completion of reaction, the reaction mixture evaporated to remove isopropyl alcohol from the reaction mixture. Resulting crude was diluted with water and extracted with DCM multiple times, the combined organic layer was concentrated to get crude mixture. The crude was purified with silica gel column chromatography by using DCM & MeOH as eluent (0-10%). The product SAPTI012S017 was confirmed with LCMS and 1H NMR; Yield 13%

LCMS [M+H]=357

### Example 19: Synthetic Procedure for the Synthesis of SAPTI012S018

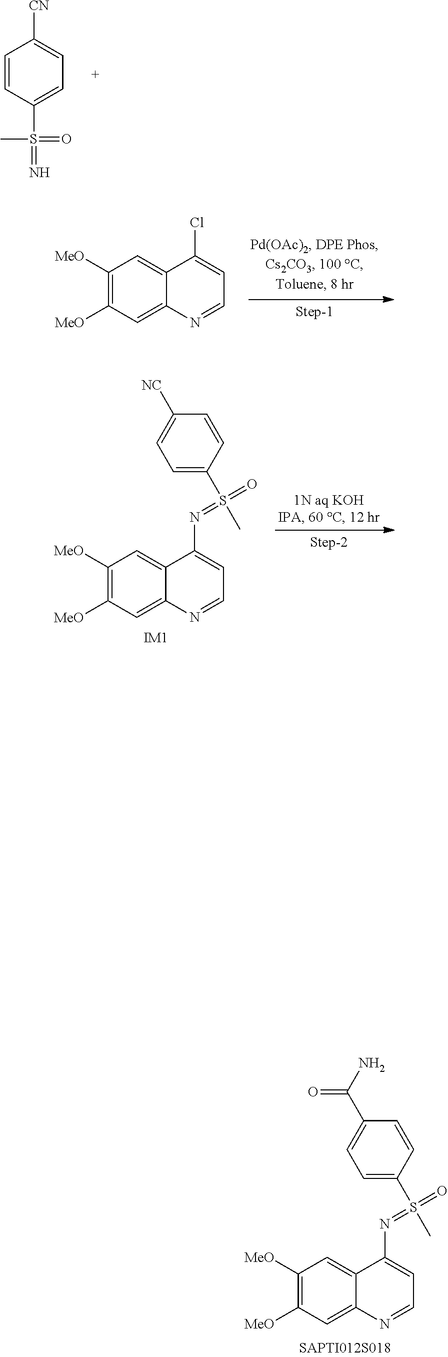

Step-1: 4-(S-methanesulfonimidoyl)benzonitrile (1.0 eq.) was dissolved in toluene (0.2M) in RBF, then DPE Phos (7.5 mol %), Cs2CO3 (1.4 eq.) followed by 4-chloro-6,7-dimethoxyquinazoline (1.5 eq.) were added, and the solution was purged with N2 for 10 mins. Then Pd(OAc)2 (5 mol %) was added and the temperature was slowly raised to 100 °C, then the reaction mixture stirred under N2 atmosphere at 100 °C for 8 hours. After completion of reaction, the reaction mixture was filtered through a pad of celite, and filtrate was evaporated and purified with silica gel column chromatography by using DCM & MeOH as eluent (0-5%). The product SAPTI012S018/IM1 was confirmed with LCMS; yield 96%.

LCMS: [M+H]=368

Step-2: The intermediate SAPTI012S018/IM1 (1.0 eq.) dissolved in Isopropyl alcohol (10 v) followed by NaOH (1 eq.) was added and resulting mixture was stirred at 80 °C for 12 hours. After completion of reaction, the reaction mixture was evaporated to remove isopropyl alcohol from the reaction mixture. Resulting crude was diluted with water and extracted with DCM multiple times, the combined organic layer was concentrated to get crude mixture. The product was purified with silica gel column chromatography by using DCM & MeOH as eluent (0-10%). The product SAPTI012S018 was confirmed with LCMS and 1H NMR; Yield 58% LCMS [M+H]=386

### Example 20: Synthetic Procedure for the Synthesis of SAPTI012S019

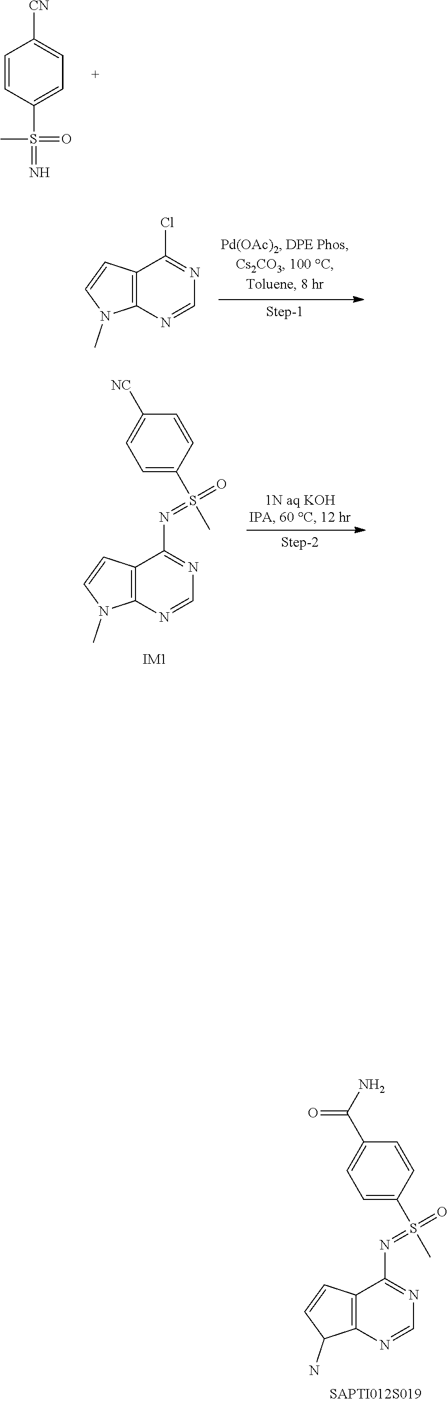

Step-1: 4-(S-methanesulfonimidoyl)benzonitrile (1.0 eq.) was dissolved in toluene (0.2M) in RBF, then DPE Phos (7.5 mol %), Cs2CO3 (1.4 eq.) followed by 4-chloro-7-methyl-7H-pyrrolo[2,3-d]pyrimidine (1.5 eq.) were added, and the solution was purged with N2 for 10 mins. Then Pd(OAc)2 (5 mol %) was added and slowly the temperature was raised to 100 °C. The reaction mixture stored under N2 atmosphere at 100 °C for 8 hours. After completion of reaction, the reaction mixture was filtered through a pad of celite, and filtrate was evaporated and purified with silica gel column chromatography by using DCM & MeOH as eluent (0-5%). The product SAPTI012S019/IM1 was confirmed with LCMS; yield 95%.

LCMS: [M+H]=312

Step-2: The intermediate SAPTI012S019/IM1 (1.0 eq.) was dissolved in Isopropyl alcohol (10 v) followed by NaOH (1 eq.) was added and the resulting mixture was stirred at 80 °C for 12 hours. After completion of reaction, the reaction mixture evaporated to remove isopropyl alcohol from the reaction mixture. Resulting crude was diluted with water and extracted with DCM multiple times, the combined organic layer was concentrated to get crude mixture. The product was purified with silica gel column chromatography by using DCM & MeOH as eluent (0-10%). The product SAPTI012S019 was confirmed with LCMS and 1H NMR; Yield 78%

LCMS [M+H]=329

### Example 21: Synthetic Procedure for the Synthesis of SAPTI012S020

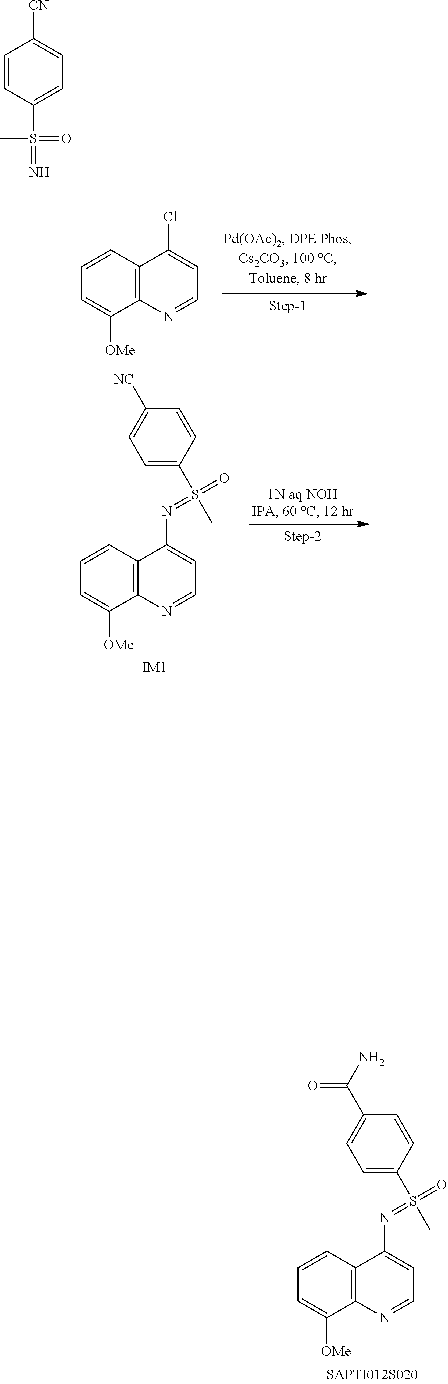

Step-1: 4-(S-methanesulfonimidoyl)benzonitrile (1.0 eq.) was dissolved in toluene (0.2M) in RBF, then DPE Phos (7.5 mol %), Cs2CO3 (1.4 eq.) followed by 4-chloro-8-methoxy quinoline (1.5 eq.) were added and the solution purged with N2 for 10 mins. Then Pd(OAc)2 (5 mol %) was added and slowly the temperature was raised to 100 °C. The reaction mixture stirred under N2 atmosphere at 100 °C for 8 hours. After completion of reaction, the reaction mixture was filtered through a pad of celite, and filtrate was evaporated and purified with silica gel column chromatography by using DCM & MeOH as eluent (0-5%). The product (SAPTI012S20/IM1) was confirmed with LCMS; yield 89%.

LCMS: [M+H]=338

Step-2: The intermediate SAPTI012S020/IM1 (1.0 eq.) was dissolved in Isopropyl alcohol (10 v) followed by NaOH (1 eq.) was added and resulting mixture was stirred at 80 °C for 12 hours. After completion of reaction, the reaction mixture evaporated to remove isopropyl alcohol from the reaction mixture. Resulting crude was diluted with water and extracted with DCM multiple times, the combined organic layer was concentrated to get crude mixture. The product was purified with silica gel column chromatography by using DCM & MeOH as eluent (0-10%). The product SAPTI012S020 was confirmed with LCMS and 1H NMR; Yield 13%

LCMS [M+H]=356

### Example 22: Synthetic Procedure for the Synthesis of SAPTI012S021

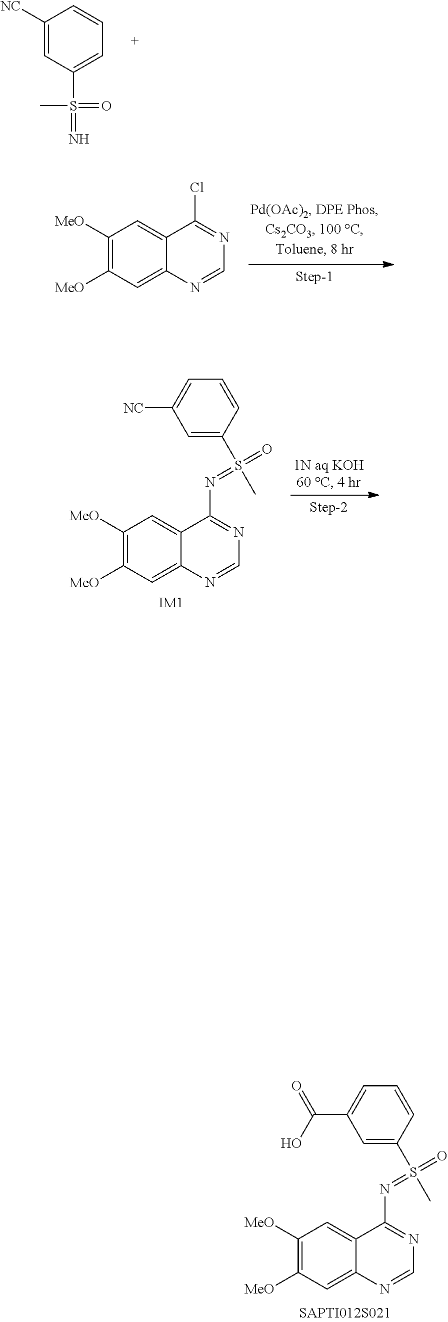

Step-1: tert-butyl {[3-(S-methanesulfonimidoyl)phenyl]methyl}carbamate (1.0 eq.) was dissolved in toluene (0.2M) in RBF, then DPE Phos (7.5 mol %), Cs2CO3 (1.4 eq.) followed by 4-chloro-6,7-dimethoxyquinazoline (1.5 eq.) were added, and the solution was purged with N2 for 10 mins. Then Pd(OAc)2 (5 mol %) was added and the temperature was raised to 100 °C. The reaction mixture was stirred under N2 atmosphere at 100 °C for 6 hours. After completion of reaction, the reaction mixture was filtered through a pad of celite, and filtrate was evaporated and purified with silica gel column chromatography by using DCM & MeOH as eluent (0-5%). The product SAPTI012S021/IM1 was confirmed with LCMS; yield 64%.

LCMS: [M+H]=369

Step-2: The intermediate SAPTI012S021/IM1 (1.0 eq.) was dissolved in 1 N KOH (10V) and resulting mixture was stirred at 60 °C for 4 hours. After completion of reaction, the reaction mixture diluted with water and extracted with DCM multiple times. Now aqueous layer was acidified with 1 N HCl till aqueous layer become acidic resulting solution extracted with DCM and this organic layer was collected and concentrated to get crude mixture, and the Product was purified with silica gel column chromatography by using DCM & MeOH as eluent (0-10%). The product SAPTI012S021 was confirmed with LCMS and 1H NMR; Yield 40%

LCMS [M+H]=388

### Example 23: Synthetic Procedure for the Synthesis of SAPTI012S022

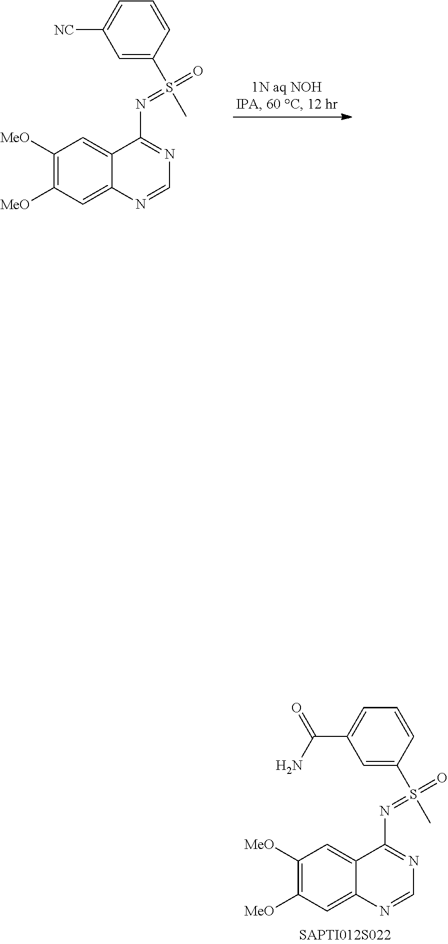 [SMILES: COc1cc2ncnc(N=S(C)(=O)c3cccc(C#N)c3)c2cc1OC ; COc1cc2ncnc(N=S(C)(=O)c3cccc(C(N)=O)c3)c2cc1OC]

The previous intermediate SAPTI012S021/IM1 (1.0 eq.) was dissolved in Isopropyl alcohol (10 v) followed by NaOH (1 eq.) was added and resulting mixture was stirred at 80 °C for 12 hours. After completion of reaction, the reaction mixture evaporated to remove isopropyl alcohol from the reaction mixture. Resulting crude was diluted with water and extracted with DCM multiple times, the combined organic layer was concentrated to get crude mixture. The product was purified with silica gel column chromatography by using DCM & MeOH as eluent (0-10%). The product SAPTI012S022 was confirmed with LCMS and 1H NMR; Yield 40%

LCMS [M+H]=387

### Example 24: Synthetic Procedure for the Synthesis of SAPTI012S023

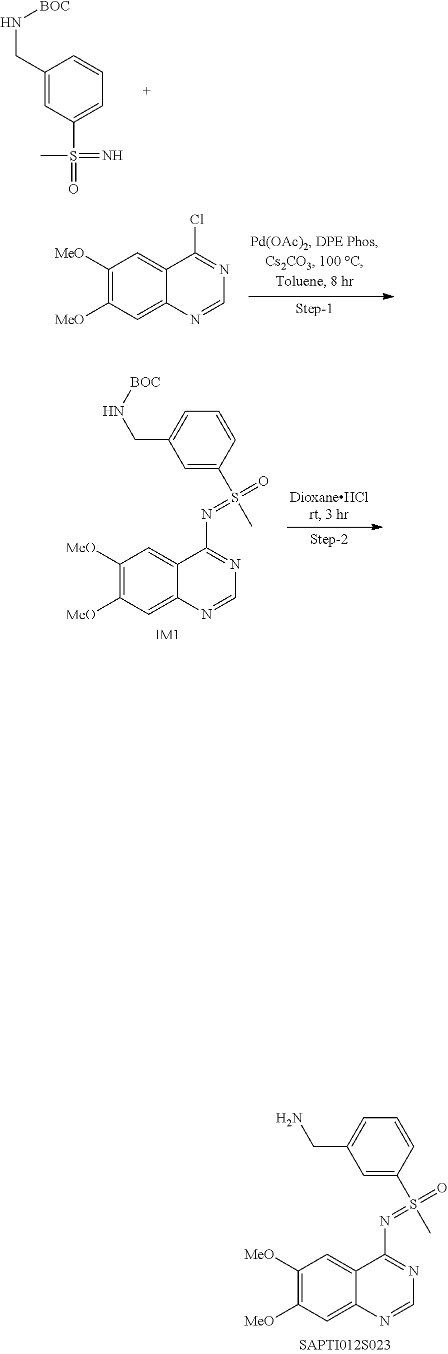

Step-1: tert-butyl {[3-(S-methanesulfonimidoyl)phenyl]methyl}carbamate (1.0 eq.) was dissolved in toluene (0.2M) in RBF, then DPE Phos (7.5 mol %), Cs2CO3 (1.4 eq.) followed by 4-chloro-6,7-dimethoxyquinazoline (1.5 eq.) were added, and the solution purged with N2 for 10 mins. Then Pd(OAc)2 (5 mol %) was added and slowly the temperature was raised to 100 °C, the reaction mixture stirred under N2 atmosphere at 100 °C for 6 hours. After completion of reaction, the reaction mixture was filtered through a pad of celite, and filtrate was evaporated and purified with silica gel column chromatography by using DCM & MeOH as eluent (0-5%). The product (SAPTI012S023/IM1) was confirmed with LCMS; yield 64%.

LCMS: [M+H]=473

Step-2: The intermediate SAPTI012S023/IM1 (1.0 eq.) was dissolved in 1,4-Dioxane (0.2M) in RBF and cooled to 0 °C with ice bath. Then dioxane HCl (10 vol.) was added dropwise and stirred for 5 hours at RT. After completion of reaction, the reaction mixture was evaporated to dryness, washed with ether. The HCl salt of product was dissolved in DCM and washed with water and NaHCO₃ solution, the organic layer was concentrated and purified with silica gel chromatography. The product SAPTI012S023 was confirmed with LCMS and used further without further purification; yield-74%.

LCMS: [M+H]=373

### Example 25: Synthetic Procedure for the Synthesis of SAPTI012S024

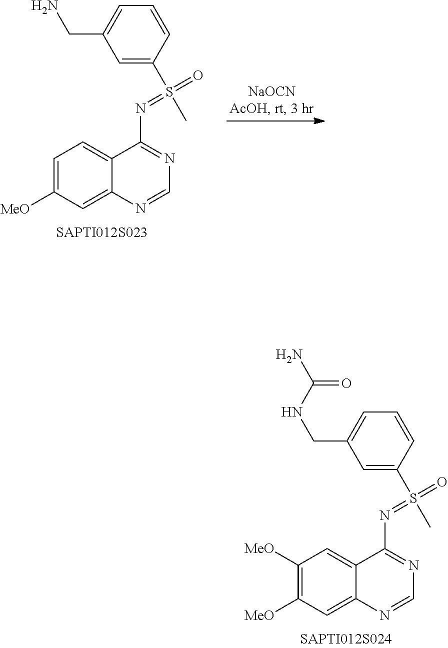 [SMILES: COc1ccc2c(N=S(C)(=O)c3cccc(CN)c3)ncnc2c1 ; COc1cc2ncnc(N=S(C)(=O)c3cccc(CNC(N)=O)c3)c2cc1OC]

Step-3: The SAPTI012S023 (1.0 eq.) was dissolved in water and AcOH (1:1) followed by sodium cyanate (1.0 eq.) was added portion wise at 0 °C and reaction was stirred at RT for 2 hours. After the completion of reaction, the reaction mixture was quenched with water and extracted with DCM multiple time, the combined organic layer was washed with brine solution and passed through anhydrous sodium sulphate and evaporated under reduced pressure to get crude and the crude was and purified by reversed phase column chromatography by using water and MeOH as an eluent (20-30%). The product SAPTI012S024 was confirmed with LCMS and 1H NMR; yield: 30%

LCMS: [M+H]=416

### Example 26: Synthetic Procedure for the Synthesis of SAPTI012S025

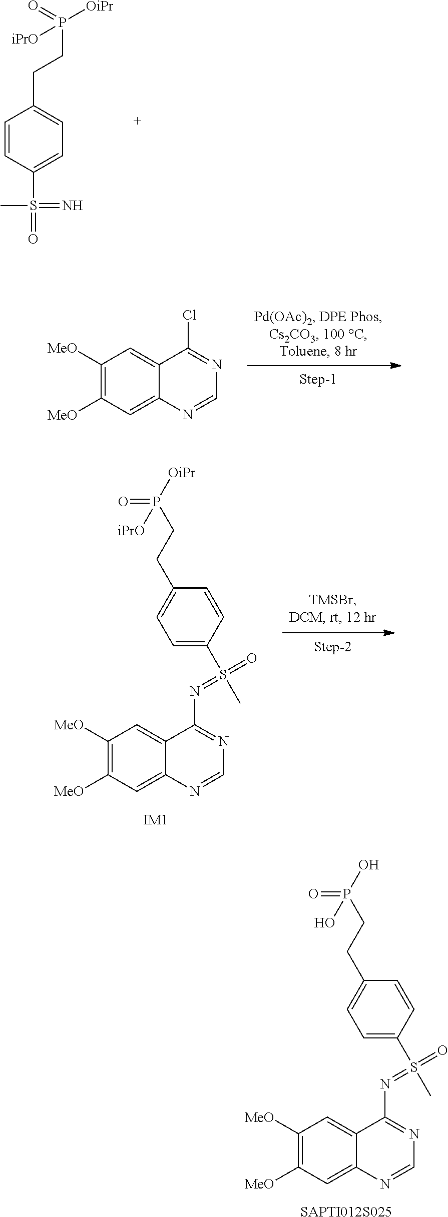

Step-1: Dipropan-2-yl {2-[4-(S-methanesulfonimidoyl)phenyl]ethyl}phosphonate (1.0 eq.) was dissolved in toluene (0.2M) in RBF, then DPE Phos (7.5 mol %), Cs2CO3 (1.4 eq.) followed by 4-chloro-6,7-dimethoxyquinazoline (1.5 eq.) were added, and the solution purged with N2 for 10 mins. Then Pd(OAc)2 was added and slowly heated to 100 °C and stirred for 6 hours under N2 atmosphere. After completion of reaction, the reaction mixture was filtered through a pad of celite, and filtrate was evaporated and purified with silica gel column chromatography by using DCM & MeOH as eluent (0-5%). The product SAPTI012S025/IM1 was confirmed with LCMS; yield 20%.

LCMS: [M+H]=536

Step-2: SAPTI012S025/IM1 (1.0 eq.) was dissolved in CHCl3 (0.2M) in a sealed tube and cooled to 0 °C with ice bath, then HBr (10 vol.) was added under N2 atmosphere and stored for 5 hours at 60 °C. After completion of reaction, the reaction mixture was evaporated to dryness, and purified with reversed phase C18 column chromatography by using 0.1% phosphoric acid in water and MeOH as eluent (10-15%). The product SAPTI012S025 was confirmed with 1H NMR and LCMS; yield-15%.

LCMS: [M+H]=452

### Example 27: Synthetic Procedure for the Synthesis of SAPTI012S026

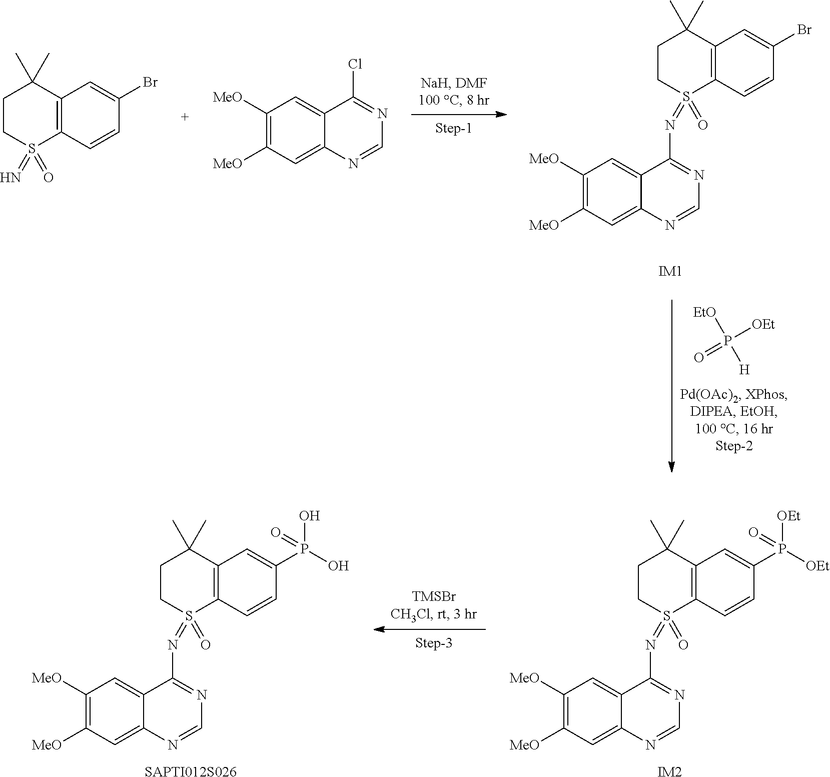

Step-1: 6-bromo-1-imino-4,4-dimethyl-1,2,3,4-tetrahydro-1-benzothiopyran-1-one (1.0 eq.) was dissolved in DMF (0.2M) then cooled to 0 °C with ice bath. Then NaH (2.0 eq.) followed by 4-chloro-6,7-dimethoxyquinazoline (1.1 eq.) were added under N2 atmosphere, and the temperature of reaction mixture was slowly raised to 100 °C and stirred over 12 hours. After completion of the reaction, the reaction mixture was quenched with water and extracted with EtOAc. The organic layer was concentrated and purified with silica gel column chromatography by using EtOAc & Hexane as eluent (05-10%). The product SAPTI012S026/IM1 was confirmed with LCMS; yield-40%.

LCMS: [M+H]=477.

Step-2: The intermediate SAPTI012S026/IM1 (1.0 eq.) was dissolved in EtOH (0.2M) in seal tube, then XPhos (7.5 mol %), DIPEA (1.4 eq.) followed by Diethyl phosphite (1.5 eq.) were added and the solution purged with N2 for 10 mins. Then Pd(OAc)2 was added and slowly the temperature was raised to 100 °C and stirred for 16 hours. After completion of reaction, reaction mixture was filtered through a pad of celite, and filtrate was evaporated and purified with column chromatography by using DCM & MeOH as eluent (0-5%). The Intermediate SAPTI012S026/IM2 was confirmed with LCMS; yield 80%.

LCMS: [M+H]=534

Step-3: The intermediate SAPTI012S026/IM2 (1.0 eq.) was dissolved in DCM (0.2M) in RBF and cooled to 0 °C with ice bath. Then Trimethylsilylbromide (10 vol.) was added under N2 atmosphere and stirred for over 12 hours at RT. After completion of reaction, reaction mixture was evaporated to dryness, purified with reversed phase column chromatography by using 0.1% phosphoric acid in water and MeOH as eluent (10-15%). The pure product SAPTI012S026 was confirmed with 1H NMR and LCMS; yield-44%.

LCMS: [M+H]=478

### Example 28: Synthetic Procedure for the Synthesis of SAPTI012S027

Step-1: dipropan-2-yl[3-(S-benzenesulfonimidoyl)propyl]phosphonate (1.0 eq.) was dissolved in toluene (0.2M) in RBF, then DPE Phos (7.5 mol %), Cs2CO3 (1.4 eq.) followed by 4-chloro-6,7-dimethoxyquinazoline (1.5 eq.) were added, and the solution purged with N2 for 10 mins. Then Pd(OAc)2 was added and slowly heated to 100 °C and stirred for 6 hours under N2 atmosphere. After completion of reaction, the reaction mixture was filtered through a pad of celite, and filtrate was evaporated and purified with silica gel column chromatography by using DCM & MeOH as eluent (0-5%). The product (SAPTI012S027/IM1) was confirmed with LCMS; yield 20%.

LCMS: [M+H]=536

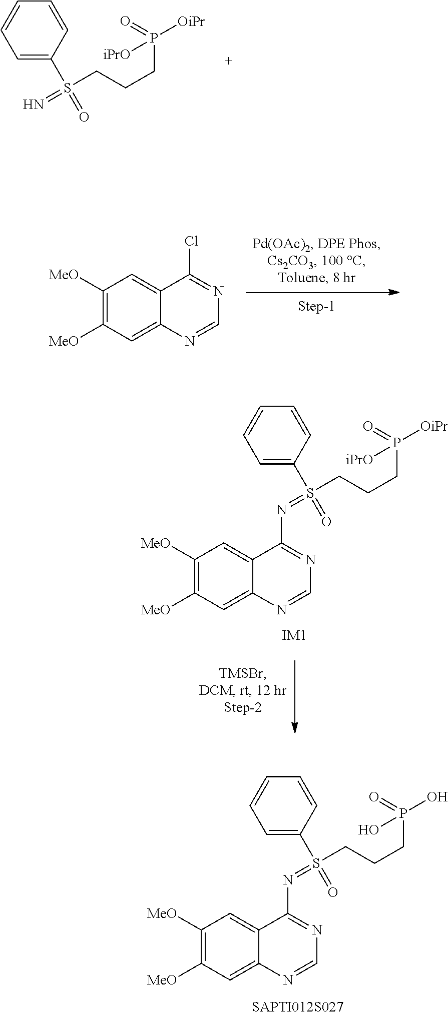

Step-2: The intermediate SAPTI012S027/IM1 (1.0 eq.) was dissolved in CHCl3 (0.2M) in a sealed tube and cooled to 0 °C with ice bath, then HBr (10V) was added under N2 atmosphere and stirred for 5 hours at 60 °C. After completion of reaction, the reaction mixture was evaporated to dryness, and purified with reversed phase C18 column chromatography by using 0.1% phosphoric acid in water and MeOH as eluent (10-15%). The product SAPTIO 12SO27 was confirmed with 1H NMR and LCMS; yield-15%.

LCMS: [M+H]=452

### Example 29: Synthetic Procedure for the Synthesis of SAPTI012S028

Step-1: 1-bromo-5-(S-methanesulfonimidoyl)naphthalene (1.0 eq.) was dissolved in THE (0.2M) in RBF, then DPE Phos (7.5 mol %), Cs2CO3 (1.4 eq.) followed by 4-chloro-6,7-dimethoxyquinazoline (1.5 eq.) were added and purged with N2 for 10 mins. Then Pd(OAc)2 was added, and the reaction mixture slowly heated to 100 °C under N2 atmosphere and stirred for 6 hours. After completion of reaction, reaction mixture was filtered through a pad of celite, and filtrate was evaporated and purified with column chromatography by using DCM & MeOH as eluent (0-5%). The product SAPTI012S028/IM1 was confirmed with LCMS; yield 65%.

LCMS: [M+H]=473

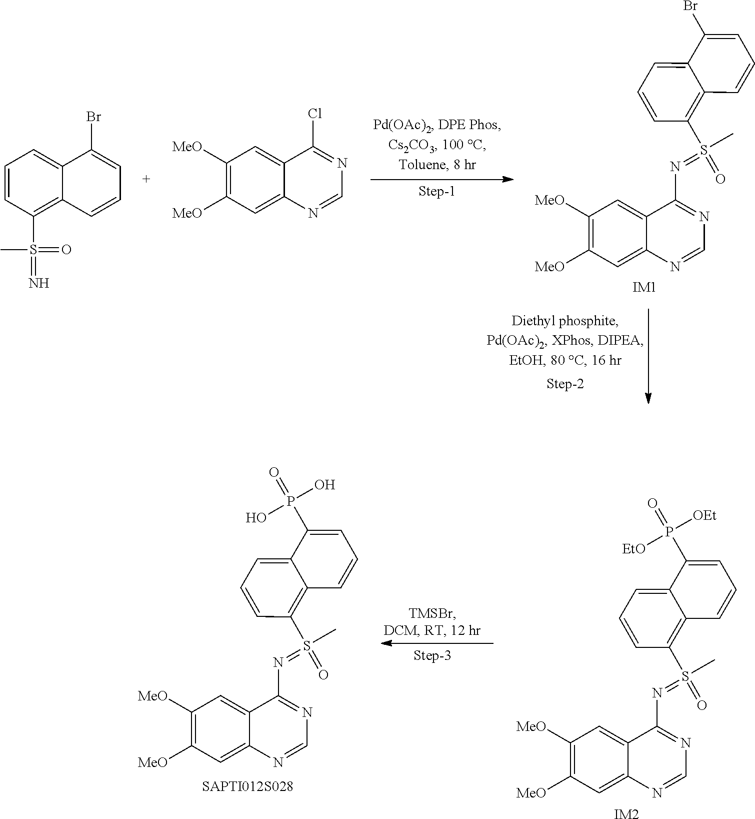

Step-2: The intermediate SAPTI012S028/IM1 (1.0 eq.) was dissolved in EtOH (0.2M) in seal tube, then XPhos (7.5 mol %), DIPEA (1.4 eq.) followed by Diethyl phosphite (1.5 eq.) were added and the solution purged with N2 for 10 mins. Then Pd(OAc)2 was added and the temperature was raised slowly to 100 °C and stirred for 16 hours. After completion of reaction, the reaction mixture was filtered through a pad of celite, and filtrate was evaporated and purified with column chromatography by using DCM & MeOH as eluent (0-5%). The Intermediate SAPTI012S028/IM2 was confirmed with LCMS; yield 80%.

LCMS: [M+H]=530

Step-3: The intermediate SAPTI012S028/IM2 (1.0 eq.) was dissolved in DCM (0.2M) in RBF and cooled to 0 °C with ice bath. Then Trimethylsilylbromide (10V) was added under N2 atmosphere and stirred for 12 hours at RT. After completion of reaction, the reaction mixture was evaporated to dryness, purified with reversed phase column chromatography by using 0.1% Phosphoric acid in water and MeOH as eluent (10-15%). The pure product SAPTI012S028 was confirmed with 1H NMR and LCMS; yield-44%.

LCMS: [M+H]=474

### Example 30: Synthetic Procedure for the Synthesis of SAPTI012S029

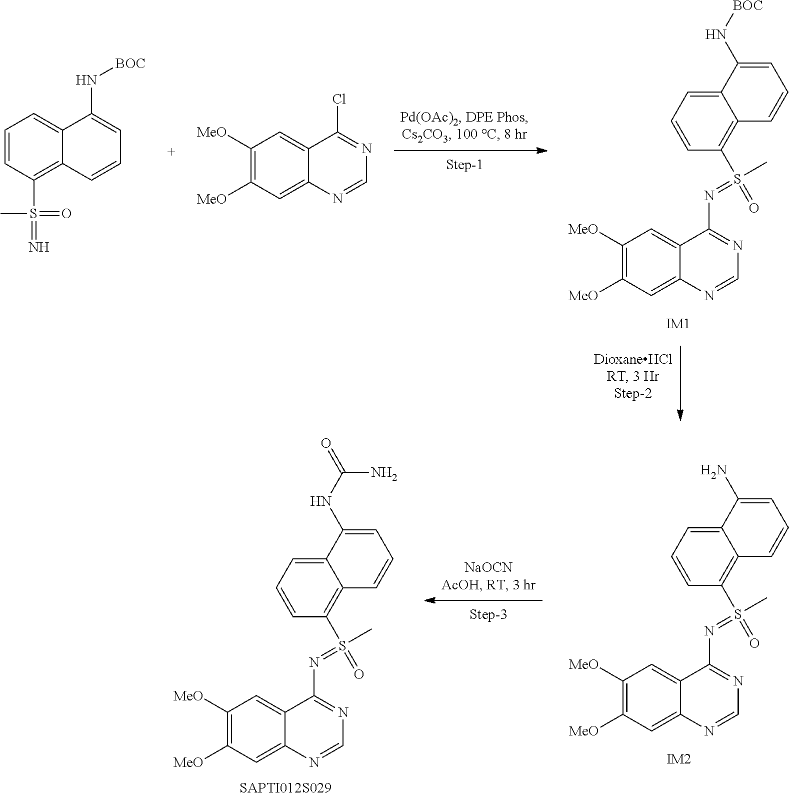

Step-1: tert-butyl[5-(S-methanesulfonimidoyl)naphthalen-1-yl]carbamate (1.0 eq.) was dissolved in toluene (0.2M) in RBF, then DPE phos (7.5 mol %), Cs2CO3 (1.4 eq.) followed by 4-chloro-6,7-dimethoxyquinazoline (1.5 eq.) were added, and the reaction mixture was purged with N2 for 10 mins. Then Pd(OAc)2 was added and slowly the reaction mixture was heated to 100 °C under N2 atmosphere and continued the stirring for 6 hours at 100 °C. After completion of reaction, the reaction mixture was filtered through a pad of celite, and filtrate was evaporated and purified with column chromatography by using DCM & MeOH as eluent (0-5%). The product SAPTI012S029/IM1 was confirmed with LCMS; yield 71%.

LCMS: [M+H]=509

Step-2: The intermediate SAPTI012S029/IM1 (1.0 eq.) was dissolved in dioxane (0.2M) in RBF and cooled to 0 °C with ice bath. Then HCl in Dioxane (10 vol.) was added and stirred for 2 hours at RT. After completion of reaction, the reaction mixture was evaporated to dryness, washed with diethyl ether, and quenched with 10% NaHCO₃ solution and extracted with DCM multiple times. The combined organic layer was concentrated and taken to the next step without purification. The product SAPTI012S029/IM2 confirmed with LCMS; yield-65%.

LCMS: [M+H]=409

Step-3: The intermediate SAPTI012S02911M2 (1.0 eq.) was dissolved in AcOH/H2O (1:1) (0.5M) in RBF followed by NaOCN (1.0 eq.) was added at 0 °C, the reaction mass was stirred at room temperature for 3 hours. After the completion of the reaction, quenched with dil. NaOH and extracted with DCM multiple times. The combined organic layer was concentrated under reduced pressure purified with neutral alumina column chromatography by using DCM & MeOH as eluent (10-15%). The pure product SAPTI012S029 was confirmed with 1H NMR and LCMS; yield-44%.

LCMS: [M+H]=452

### Example 31: Synthetic Procedure for the Synthesis of SAPTI012S030

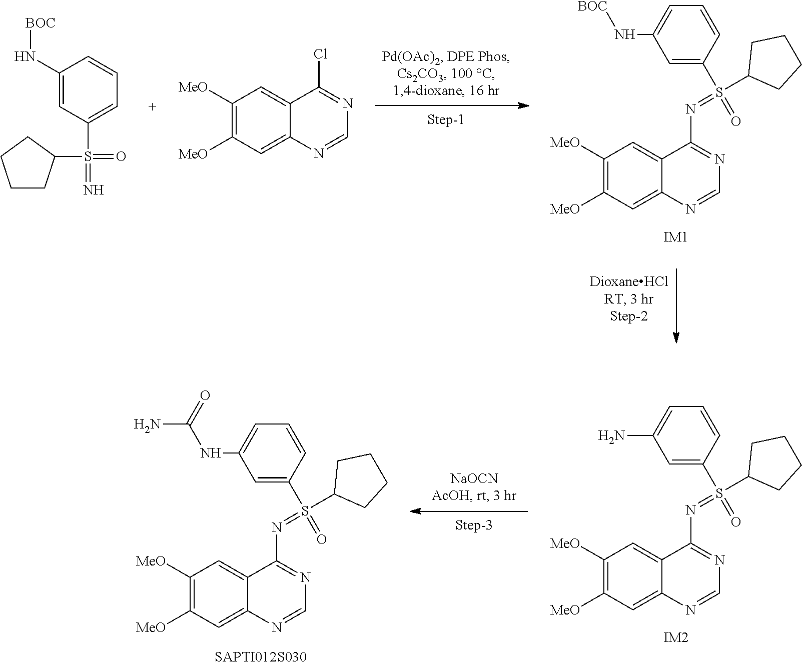

Step-1: tert-butyl N-[3-(cyclopentylsulfonimidoyl)phenyl]carbamate (1.0 eq.) was dissolved in toluene (0.2M) in RBF, then DPE Phos (7.5 mol %), Cs2CO3 (1.4 eq.) followed by 4-chloro-6,7-dimethoxyquinazoline (1.5 eq.) were added, and the solution was purged with N2 for 10 mins. Then Pd(OAc)2 (5 mol %) was added and the temperature was raised to 100 °C. The reaction mixture was stirred under N2 atmosphere at 100 °C for 6 hours. After completion of reaction, the reaction mixture was filtered through a pad of celite, and filtrate was evaporated and purified with silica gel column chromatography by using DCM & MeOH as eluent (0-5%). The product (SAPTI012S030/IM1) was confirmed with LCMS; yield 63%.

LCMS: [M+H]=513

Step-2: SAPTI012S030/IM1 (1.0 eq.) was dissolved in 1,4-Dioxane (0.2M) in RBF and cooled to 0 °C with ice bath. Then Dioxane HCl (10 vol.) was added dropwise and stirred for 5 hours at RT. After completion of reaction, the reaction mixture was evaporated to dryness, washed with ether. The HCl salt of SAPTI012S030/IM2 was confirmed with LCMS and used without further purification; yield-72%.

Step-3: The HCl salt of SAPTI012S030/IM2 (1.0 eq.) was dissolved in water and AcOH (1:1) followed by sodium cyanate (1 eq.) was added portion wise at 0 °C and reaction was stirred at RT for 2 hours. After the completion of reaction, the reaction mixture was quenched with water and extracted with DCM multiple time, the combined organic layer was washed with brine solution and passed through anhydrous sodium sulphate and evaporated under reduced pressure and purified by reversed phase column chromatography by using water and MeOH as an eluent (20-30%). The product SAPTI012S030 was confirmed with LCMS and 1H NMR; yield: 30%

LCMS: [M+H]=456

### Example 32: Synthetic Procedure for the Synthesis of SAPTI012S031

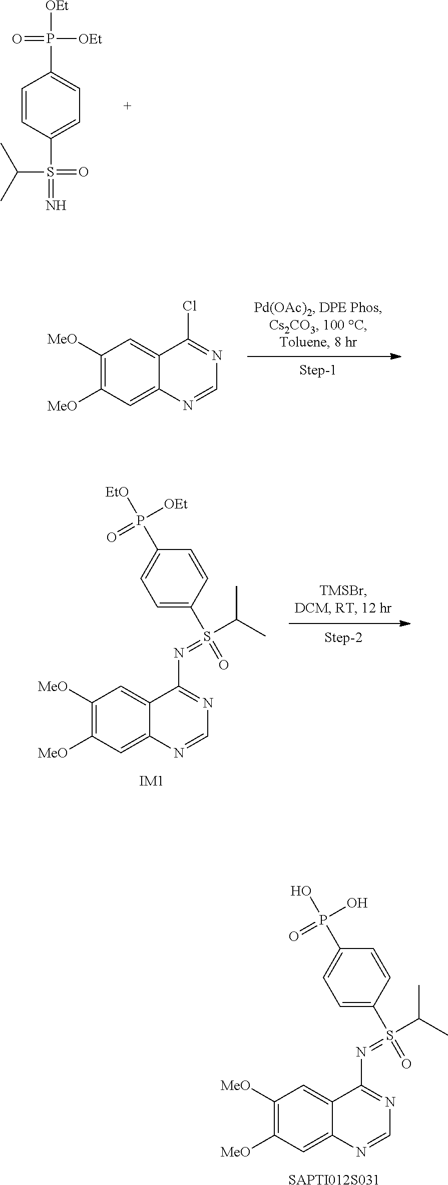

Step-1: diethyl {4-[S-(propane-2-)sulfonimidoyl]phenyl}phosphonate (1.0 eq.) was dissolved in toluene (0.2M) in RBF, then DPE Phos (7.5 mol %), Cs2CO3 (1.4 eq.) followed by 4-chloro-6,7-dimethoxyquinazoline (1.5 eq.) were added, and the solution purged with N2 for 10 mins. Then Pd(OAc)2 was added and the reaction mixture was slowly heated to 100 °C and stirred for 6 hours under N2 atmosphere. After completion of reaction, the reaction mixture was filtered through a pad of celite, and filtrate was evaporated and purified with silica gel column chromatography by using DCM & MeOH as eluent (0-5%). The product SAPTI012S031/IM1 was confirmed with LCMS; yield 20%.

LCMS: [M+H]=508

Step-2: The intermediate SAPTI012S031/IM1 (1.0 eq.) was dissolved in CHCl3 (0.2M) in a sealed tube and cooled to 0 °C with ice bath, then HBr (10 vol.) was added under N2 atmosphere and stirred for 5 hours at 60 °C. After completion of reaction, reaction mixture was evaporated to dryness, and purified with reversed phase C18 column chromatography by using 0.1% phosphoric acid in water and MeOH as eluent (10-15%). The product SAPTI012S031 was confirmed with 1H NMR and LCMS; yield-15%.

LCMS: [M+H]=452

### Example 33: Synthetic Procedure for the Synthesis of SAPTI012S032

Step-1: tert-butyl N-[3-(propan-2-ylsulfonimidoyl)phenyl]carbamate (1.0 eq.) was dissolved in toluene (0.2M) in RBF, then DPE Phos (7.5 mol %), Cs2CO3 (1.4 eq.) followed by 4-chloro-6,7-dimethoxyquinazoline (1.5 eq.) were added, and the solution was purged with N2 for 10 mins. Then Pd(OAc)2 (5 mol %) was added and slowly the temperature was raised to 100 °C. The reaction mixture stirred under N2 atmosphere at 100 °C for 6 hours. After completion of reaction, the reaction mixture was filtered through a pad of celite, and filtrate was evaporated and purified with silica gel column chromatography by using DCM & MeOH as eluent (0-5%). The product SAPTI012S032/IM1 was confirmed with LCMS; yield 30%.

LCMS: [M+H]=485

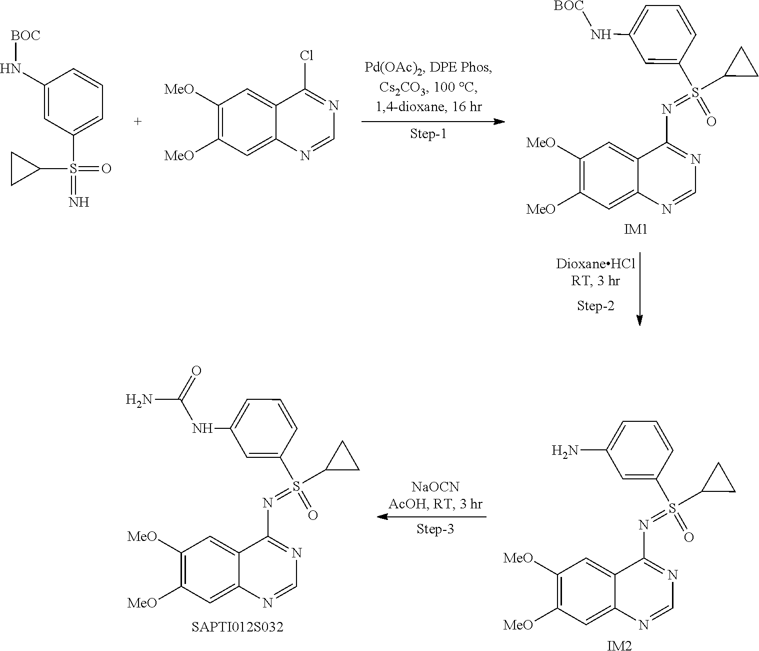

Step-2: The intermediate SAPTI012S032/IM1 (1.0 eq.) was dissolved in 1,4-Dioxane (0.2M) in RBF and cooled to 0 °C with ice bath. Then Dioxane HCl (10 vol.) was added dropwise and stirred for over 5 hours at RT. After completion of reaction, the reaction mixture was evaporated to dryness, washed with ether. The HCl salt of SAPTI012S032/IM2 was confirmed with LCMS and used without further purification; yield-72%.

Step-3: The HCl salt of SAPTI012S032/IM2 (1.0 eq.) was dissolved in water and AcOH (1:1) followed by sodium cyanate (1 eq.) was added portion wise at 0 °C and reaction was stirred at RT for 2 hours. After the completion of reaction, the reaction mixture was quenched with water and extracted with DCM multiple time, the combined organic layer was washed with brine solution and passed through anhydrous sodium sulphate and evaporated under reduced pressure and purified by reversed phase column chromatography by using water and MeOH as an eluent (20-30%). The product SAPTI012S032 was confirmed with LCMS and 1H NMR; yield: 65%

LCMS: [M+H]=428

### Example 34: Synthetic Procedure for the Synthesis of SAPTI012S033

Step-1: tert-butyl N-[3-(propan-2-ylsulfonimidoyl)phenyl]carbamate (1.0 eq.) was dissolved in toluene (0.2M) in RBF, then DPE Phos (7.5 mol %), Cs2CO3 (1.4 eq.) followed by 4-chloro-6,7-dimethoxyquinazoline (1.5 eq.) were added, and the solution was purged with N2 for 10 mins. Then Pd(OAc)2 (5 mol %) was added the temperature was raised slowly to 100 °C. The reaction mixture was stirred under N2 atmosphere at 100 °C for 6 hours. After completion of reaction, the reaction mixture was filtered through a pad of celite, and filtrate was evaporated and purified with silica gel column chromatography by using DCM & MeOH as eluent (0-5%). The product SAPTI012S033/IM1 was confirmed with LCMS; yield 61%.

LCMS: [M+H]=487

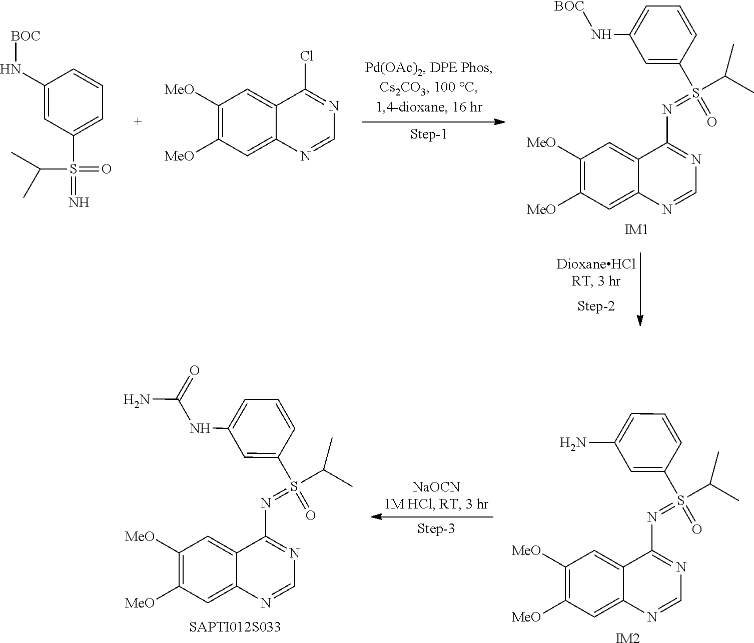

Step-2: The intermediate SAPTI012S033/IM1 (1.0 eq.) was dissolved in 1,4-Dioxane (0.2M) in RBF and cooled to 0 °C with ice bath. Then Dioxane HCl (10 vol.) was added dropwise and stirred for over 5 hours at RT. After completion of reaction, the reaction mixture was evaporated to dryness, washed with ether. The HCl salt of SAPTI012S033/IM2 was confirmed with LCMS and used without further purification; yield-72%.

Step-3: The HCl salt of SAPTI012S033/IM2 (1.0 eq.) was dissolved in water and AcOH (1:1) followed by sodium cyanate (1 eq.) was added portion wise at 0 °C and reaction was stirred at RT for 2 hours. After the completion of reaction, the reaction mixture was quenched with water and extracted with DCM multiple times, the combined organic layer was washed with brine solution and passed through anhydrous sodium sulphate and evaporated under reduced pressure and purified by reversed phase column chromatography by using water and MeOH as an eluent (20-30%). The product SAPTI012S033 was confirmed with LCMS and 1H NMR; yield: 30%

LCMS: [M+H]=430

### Example 35: Synthetic Procedure for the Synthesis of SAPTI012S034

Step-1: tert-butyl[4-(S-benzenesulfonimidoyl)phenyl]carbamate (1.0 eq.) was dissolved in toluene (0.2M) in RBF, then DPE Phos (7.5 mol %), Cs2CO3 (1.4 eq.) followed by 4-chloro-6,7-dimethoxyquinazoline (1.5 eq.) were added, and the solution was purged with N2 for 10 mins. Then Pd(OAc)2 (5 mol %) was added and the temperature was slowly raised to 100 °C. The reaction mixture was stirred under N2 atmosphere at 100 °C for 16 hours. After completion of reaction, the reaction mixture was filtered through a pad of celite, and filtrate was evaporated and purified with silica gel column chromatography by using DCM & MeOH as eluent (0-5%). The product SAPTI012S034/IM1 was confirmed with LCMS; yield 30%.

LCMS: [M+H]=521

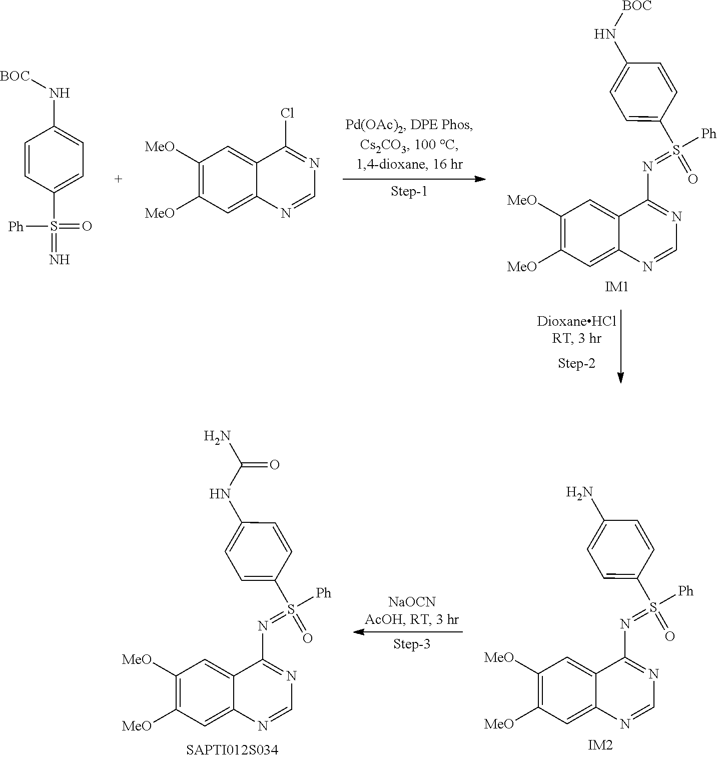

Step-2: The intermediate SAPTI012S034/IM1 (1.0 eq.) was dissolved in 1,4-Dioxane (0.2M) in RBF and cooled to 0 °C with ice bath. Then Dioxane.HCl (10 vol.) was added dropwise and stirred for 5 hours at RT. After completion of reaction, the reaction mixture was evaporated to dryness, washed with ether. The HCl salt of SAPTI012S034/IM2 was confirmed with LCMS and used without further purification; yield-60%.

Step-3: The HCl salt of SAPTI012S034/IM2 (1.0 eq.) was dissolved in water and AcOH (1:1) followed by sodium cyanate (1 eq.) was added portion wise at 0 °C and reaction was stirred at RT for 2 hours. After the completion of reaction, the reaction mixture was quenched with water and extracted with DCM multiple times, the combined organic layer was washed with brine solution and passed through anhydrous sodium sulphate and evaporated under reduced pressure, followed by the crude was purified wit reverse phase column chromatography by using water and MeOH as an eluent (20-30%). The product SAPTI012S034 was confirmed with LCMS and 1H NMR; yield: 55%

LCMS: [M+H]=464

### Example 36: Synthetic Procedure for the Synthesis of SAPTI012S035

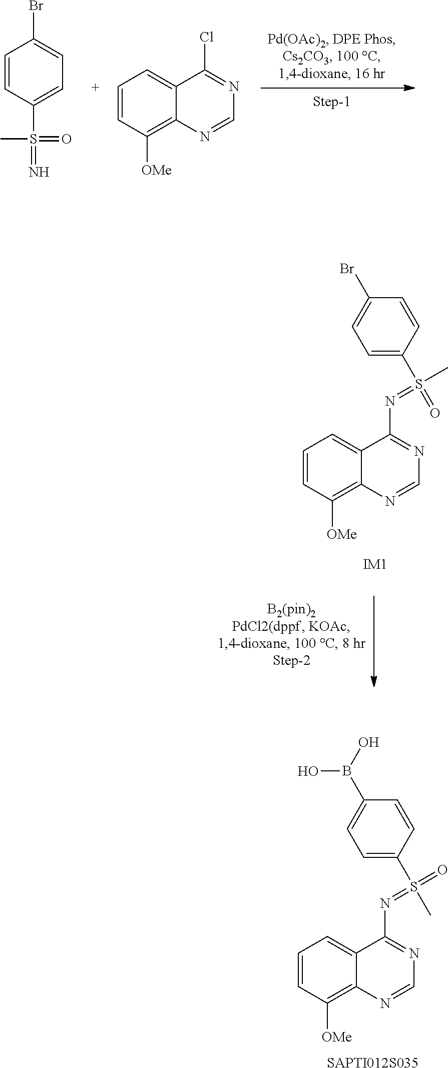

Step-1: 1-bromo-4-(S-methanesulfonimidoyl)benzene (1.0 eq.) was dissolved in toluene (0.2M) in RBF, then DPE Phos (7.5 mol %), Cs2CO3 (1.4 eq.) followed by 4-chloro-8-methoxyquinazoline (1.5 eq.) were added and the solution was purged with N2 for 10 mins. Then Pd(OAc)2 (5 mol %) was added and the temperature was raised to 100 °C, and the reaction mixture was stirred under N2 atmosphere at 100 °C for 16 hours. After completion of reaction, the reaction mixture was filtered through a pad of celite, and the filtrate was evaporated and purified with silica gel column chromatography by using DCM & MeOH as eluent (0-5%). The product SAPTI012S035/IM1 was confirmed with LCMS; yield 35%.

LCMS: [M+H]=393

Step-2: The intermediate SAPTI012S035/IM1 (1.0 eq.) was dissolved in dry 1,4-dioxane (0.2M), then B2(Pin)2 (1.4 eq.) KOAc (3.5 eq.), followed by PdCl2(dppf) complex (5 mol %) were added and the reaction mixture was purged with N2 for 10 mins, later slowly raise the temperature to 100 °C, and stirred at same temperature for 5 hours. After completion of reaction, the reaction mixture was filtered through a pad of celite, and filtrate was evaporated and purified with silica gel column chromatography by using DCM & MeOH as eluent (0-5%). The product SAPTI012S035 was confirmed with LCMS, and 1H NMR; yield 40%.

LCMS: [M+H]=358.

### Example 37: Synthetic Procedure for the Synthesis of SAPTI012S036

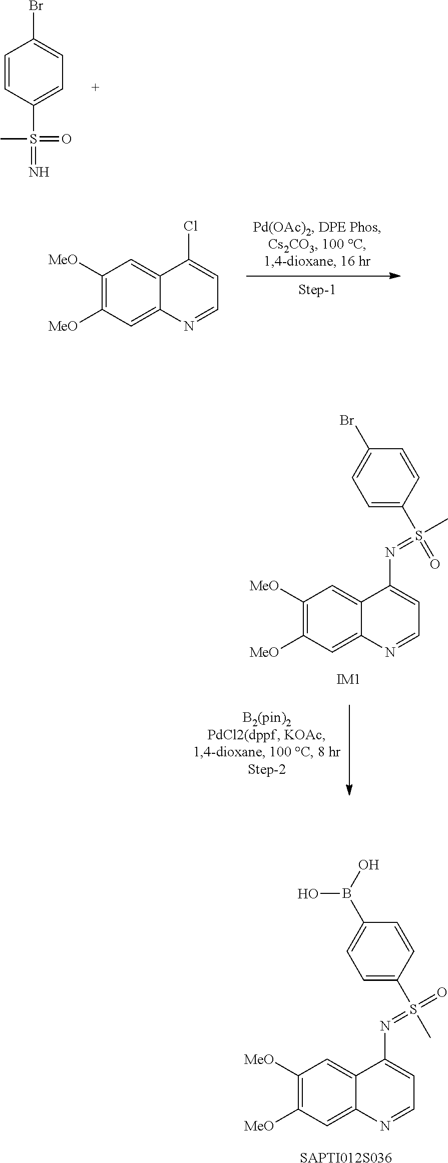

Step-1: 1-bromo-4-(S-methanesulfonimidoyl)benzene (1.0 eq.) was dissolved in toluene (0.2M) in RBF, then DPE Phos (7.5 mol %), Cs2CO3 (1.4 eq.) followed by 4-bromo-6,7-dimethoxyquinoline (1.5 eq.) were added and the solution was purged with N2 for 10 mins, then Pd(OAc)2 (5 mol %) was added and temperature was raised slowly to 100 °C. The reaction mixture was stirred under N2 atmosphere at 100 °C for 16 hours. After completion of reaction, the reaction mixture was filtered through a pad of celite, and filtrate was evaporated and purified with silica gel column chromatography by using DCM & MeOH as eluent (0-5%). The product SAPTI012S036/IM1 was confirmed with LCMS; yield 65%.

LCMS: [M+H]=422

Step-2: The intermediate SAPTI012S036/IM1 (1.0 eq.) was dissolved in dry dioxane (0.2M), then B2(Pin)2 (1.4 eq.) KOAc (3.5 eq.), followed by PdCl2(dppf) complex (5 mol %) were added and the reaction mixture purged with N2 for 10 mins, later slowly raise the temperature to 100 °C, and stirred at same temperature for 5 hours. After completion of reaction, the reaction mixture was filtered through a pad of celite, and filtrate was evaporated and purified with silica gel column chromatography by using DCM & MeOH as eluent (0-5%). The product SAPTI012S036 was confirmed with LCMS, and 1H NMR; yield 45%.

LCMS: [M+H]=387.

### Example 38: Synthetic Procedure for the Synthesis of SAPTI012S037

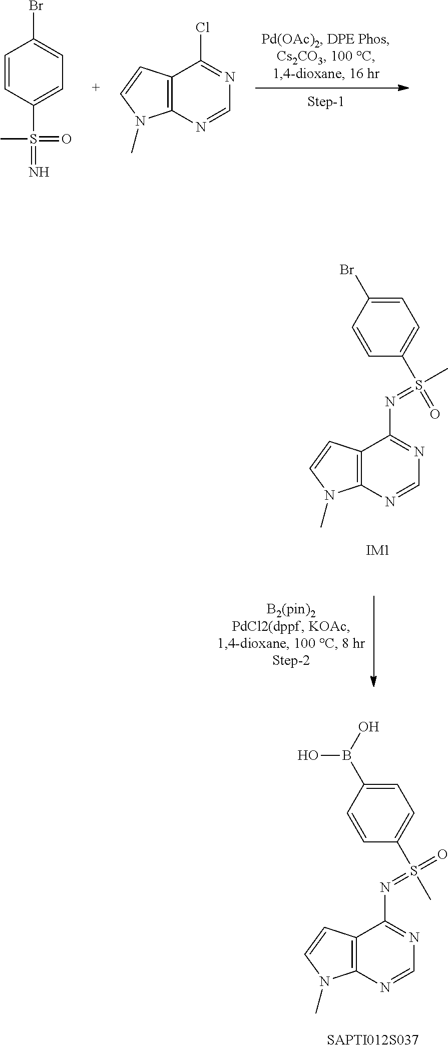

Step-1: 1-bromo-4-(S-methanesulfonimidoyl)benzene (1.0 eq.) was dissolved in toluene (0.2M) in RBF, then DPE Phos (7.5 mol %), Cs2CO3 (1.4 eq.) followed by 4-chloro-7-methyl-7H-pyirolo[2,3-d]pyrimidine (1.5 eq.) were added and the solution was purged with N2 for 10 mins, then Pd(OAc)2 (5 mol %) was added and temperature was raised slowly to 100 °C. The reaction mixture stirred under N2 atmosphere at 100 °C for 16 hours. After completion of reaction, the reaction mixture was filtered through a pad of celite, and filtrate was evaporated and purified with silica gel column chromatography by using DCM & MeOH as eluent (0-5%). The product SAPTI012S037/IM1 was confirmed with LCMS; yield 43%.

LCMS: [M+H]=365

Step-2: The intermediate SAPTI012S037/IM1 (1.0 eq.) was dissolved in dry dioxane (0.2M), then B2(Pin)2 (1.4 eq.) KOAc (3.5 eq.), followed by PdCl2(dppf) complex (5 mol %) were added and the reaction mixture was purged with N2 for 10 mins, later slowly raise the temperature to 100 °C, and stirred at same temperature for 5 hours. After completion of reaction, the reaction mixture was filtered through a pad of celite, and filtrate was evaporated and purified with silica gel column chromatography by using DCM & MeOH as eluent (0-5%). The product SAPTI012S037 was confirmed with LCMS, and 1H NMR; yield 55%.

LCMS: [M+H]=331.

### Example 39: Synthetic Procedure for the Synthesis of SAPTI012S038

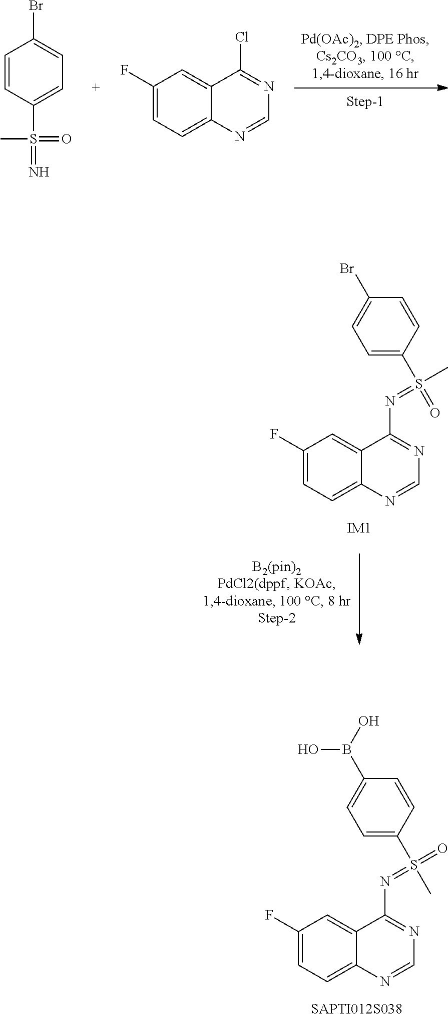

Step-1: 1-bromo-4-(S-methanesulfonimidoyl)benzene (1.0 eq.) was dissolved in toluene (0.2M) in RBF, then DPE Phos (7.5 mol %), Cs2CO3 (1.4 eq.) followed by 4-chloro-6-fluoroquinazoline (1.5 eq.) were added and the solution was purged with N2 for 10 mins, then Pd(OAc)2 (5 mol %) was added and temperature was raised slowly to 100 °C. The reaction mixture stirred under N2 atmosphere at 100 °C for 16 hours. After completion of reaction, the reaction mixture was filtered through a pad of celite, and filtrate was evaporated and purified with silica gel column chromatography by using DCM & MeOH as eluent (0-5%). The product SAPTI012S038/IM1 was confirmed with LCMS; yield 34%.

LCMS: [M+H]=381

Step-2: The intermediate SAPTI012S038/IM1 (1.0 eq.) was dissolved in dry dioxane (0.2M), then B2(Pin)2 (1.4 eq.) KOAc (3.5 eq.), followed by PdCl2(dppf) complex (5 mol %) were added and the reaction mixture purged with N2 for 10 mins, later slowly raise the temperature to 100 °C, and stirred at same temperature for 5 hours. After completion of reaction, the reaction mixture was filtered through a pad of celite, and filtrate was evaporated and purified with silica gel column chromatography by using DCM & MeOH as eluent (0-5%). The product SAPTI012S038 was confirmed with LCMS, and 1H NMR; yield 50%.

LCMS: [M+H]=346.

### Example 40: Synthetic Procedure for the Synthesis of SAPTI012S039

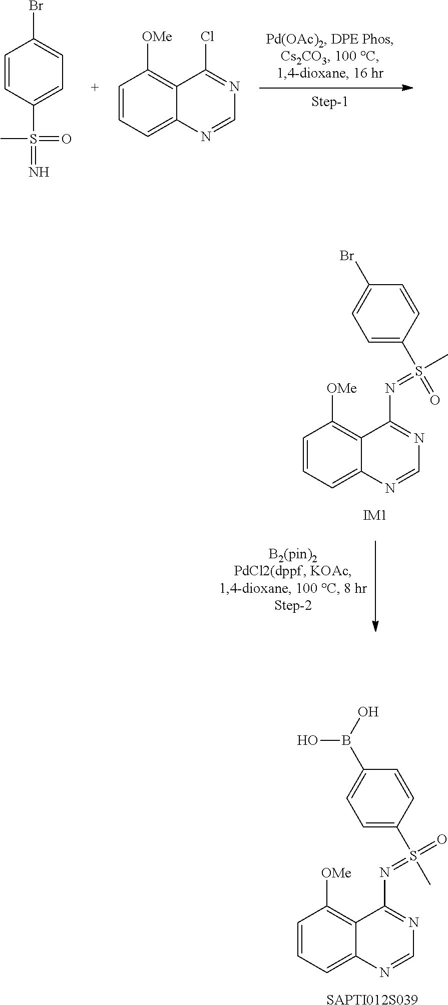

Step-1: 1-bromo-4-(S-methanesulfonimidoyl)benzene (1.0 eq.) was dissolved in toluene (0.2M) in RBF, then DPE Phos (7.5 mol %), Cs2CO3 (1.4 eq.) followed by 4-chloro-5-methoxyquinazoline (1.5 eq.) were added and the solution was purged with N2 for 10 mins, then Pd(OAc)2 (5 mol %) was added and temperature was raised slowly to 100 °C. The reaction mixture was stirred under N2 atmosphere at 100 °C for 16 hours. After completion of reaction, the reaction mixture was filtered through a pad of celite, and filtrate was evaporated and purified with silica gel column chromatography by using DCM & MeOH as eluent (0-5%). The product SAPTI012S039/IM1 was confirmed with LCMS; yield 76%.

LCMS: [M+H]=393

Step-2: The intermediate SAPTI012S039/IM1 (1.0 eq.) was dissolved in dry dioxane (0.2M), then B2(Pin)2 (1.4 eq.), KOAc (3.5 eq.), followed by PdCl2(dppf) complex (5 mol %) were added and the reaction mixture was purged with N2 for 10 mins, later slowly raise the temperature to 100 °C, and stirred at same temperature for 5 hours. After completion of reaction, the reaction mixture was filtered through a pad of celite, and filtrate was evaporated and purified with silica gel column chromatography by using DCM & MeOH as eluent (0-5%). The product SAPTI012S039 was confirmed with LCMS, 1H NMR; yield 25%.

LCMS: [M+H]=358.

### Example 41: Synthetic Procedure for the Synthesis of SAPTI012S040

Step-1: 1-bromo-4-(S-methanesulfonimidoyl)benzene (1.0 eq.) was dissolved in toluene (0.2M) in RBF, then DPE Phos (7.5 mol %), Cs2CO3 (1.4 eq.) followed by 4-chloro-8-methoxypyrido[3,4-d]pyrimidine (1.5 eq.) were added and the solution was purged with N2 for 10 mins, then Pd(OAc)2 (5 mol %) was added and temperature was raised slowly to 100 °C. The reaction mixture stored under N2 atmosphere at 100 °C for 16 hours. After completion of reaction, the reaction mixture was filtered through a pad of celite, and filtrate was evaporated and purified with silica gel column chromatography by using DCM & MeOH as eluent (0-5%). The product SAPTI012S040/IM1 was confirmed with LCMS; yield 34%.

LCMS: [M+H]=394

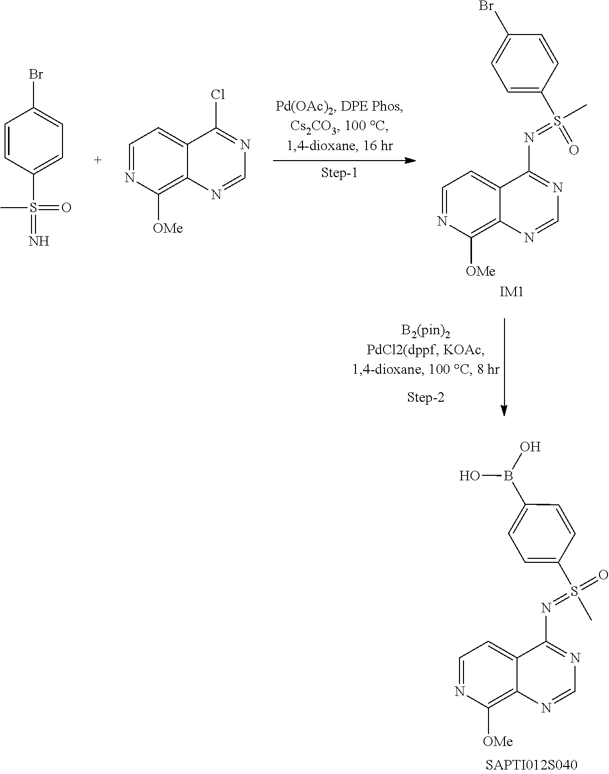

Step-2: The intermediate SAPTI012S040/IM1 (1.0 eq.) was dissolved in dry dioxane (0.2M), then B2(Pin)2 (1.4 eq.) KOAc (3.5 eq.), followed by PdCl2(dppf) complex (5 mol %) were added and the reaction mixture was purged with N2 for 10 mins, later slowly raise the temperature to 100 °C, and stored at same temperature for 5 hours. After completion of reaction, the reaction mixture was filtered through a pad of celite, and filtrate was evaporated and purified with silica gel column chromatography by using DCM & MeOH as eluent (0-5%). The product SAPTI012S040 was confirmed with LCMS, and 1H NMR; yield 30%.

LCMS: [M+H]=359.

### Example 42: Synthetic Procedure for the Synthesis of SAPTI012S041

Step-1: 1-bromo-4-(S-methanesulfonimidoyl)benzene (1.0 eq.) was dissolved in toluene (0.2M) in RBF, then DPE Phos (7.5 mol %), Cs2CO3 (1.4 eq.) followed by 4-chloroquinazoline (1.5 eq.) were added and the solution was purged with N2 for 10 mins, then Pd(OAc)2 (5 mol %) was added and temperature was raised slowly to 100 °C. The reaction mixture stirred under N2 atmosphere at 100 °C for 16 hours. After completion of reaction, the reaction mixture was filtered through a pad of celite, and filtrate was evaporated and purified with silica gel column chromatography by using DCM & MeOH as eluent (0-5%). The product SAPTI012S041/IM1 was confirmed with LCMS; yield 92%.

LCMS: [M+H]=363

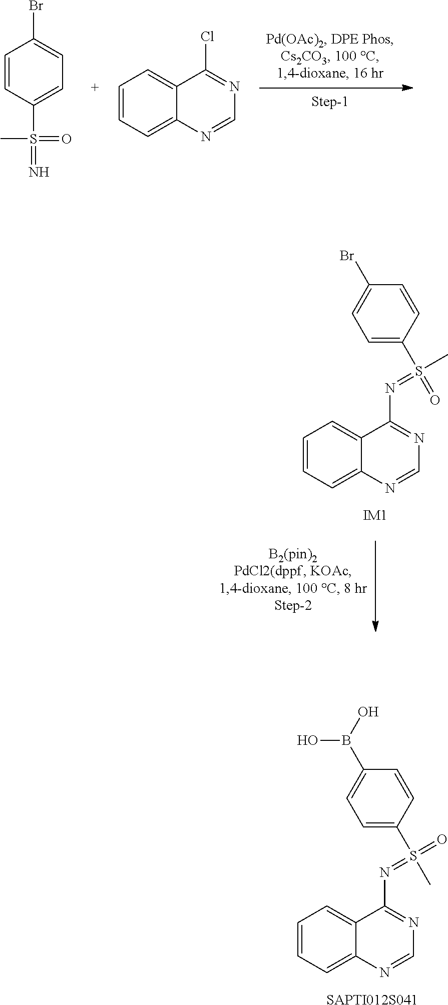

Step-2: The intermediate SAPTI012S041/IM1 (1.0 eq.) was dissolved in dry dioxane (0.2M), then B2(Pin)2 (1.4 eq.) KOAc (3.5 eq.), followed by PdCl2(dppf) complex (5 mol %) were added and the reaction mixture was purged with N2 for 10 mins, later temperature was raised slowly to 100 °C, and stirred at same temperature for 5 hours. After completion of reaction, the reaction mixture was filtered through a pad of celite, and filtrate was evaporated and purified with silica gel column chromatography by using DCM & MeOH as eluent (0-5%). The product SAPTI012S041 was confirmed with LCMS, 1H NMR; yield 25%.

LCMS: [M+H]=328.

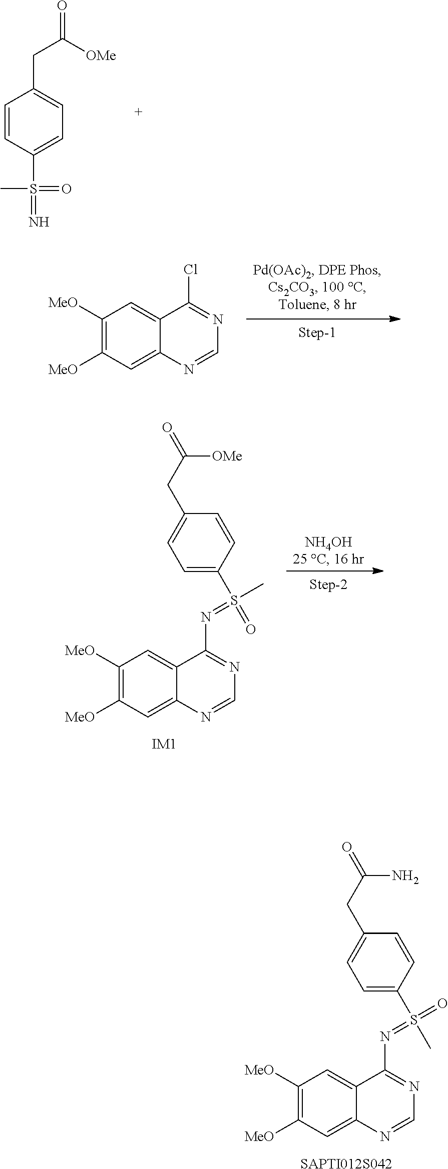

Step-1: Methyl[4-(S-methanesulfonimidoyl)phenyl]acetate (1.0 eq.) was dissolved in toluene (0.2M) in RBF, then DPE Phos (7.5 mol %), Cs2CO3 (1.4 eq.) followed by 4-chloro-8-methoxyquinazoline (1.5 eq.) were added and the solution was purged with N2 for 10 mins, then Pd(OAc)2 (5 mol %) was added and temperature was raised slowly to 100 °C. The reaction mixture was stirred under N2 atmosphere at 100 °C for 6 hours. After completion of reaction, the reaction mixture was filtered through a pad of celite, and filtrate was evaporated and purified with silica gel column chromatography by using DCM & MeOH as eluent (0-5%). The product SAPTI012S042/IM1 was confirmed with LCMS; yield 79%.

LCMS: [M+H]=416

Step-2: A (0.2M) Solution of the SAPTI012S042/IM1 in Aq. NH₄OH (28-30%) was stored at room temperature (25 °C) for 16 hours. Then the solvent was evaporated to afford crude reaction mixture and the crude was purified with flash column chromatography by using neutral alumina as stationary phase. (0-5%) MeOH in DCM used as solvents. The Product SAPTI012S042 was confirmed with LCMS, and 1H NMR; yield 34%.

LCMS: [M+H]=401

### Example 44: Synthetic Procedure for the Synthesis of SAPTI012S043

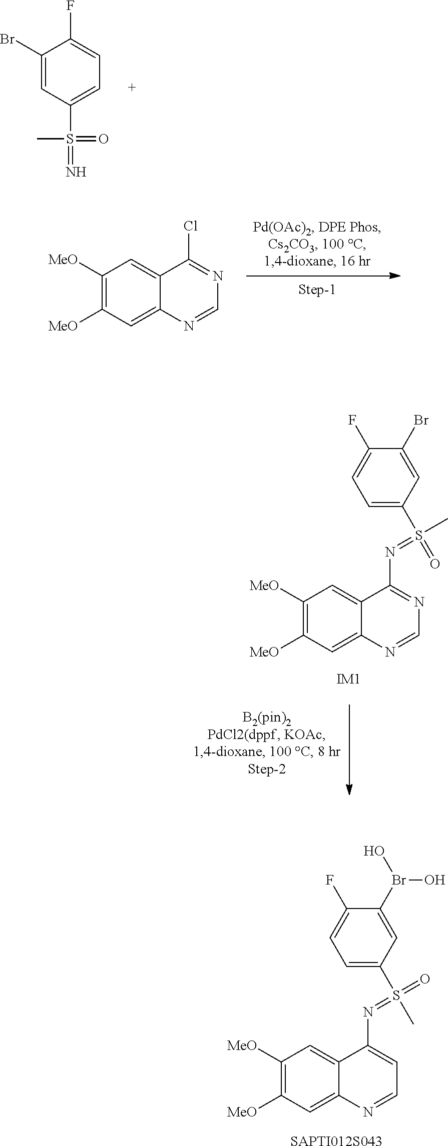

Step-1: 2-bromo-1-fluoro-4-(S-methanesulfonimidoyl)benzene (1.0 eq.) was dissolved in toluene (0.2M) in RBF, then DPE Phos (7.5 mol %), Cs2CO3 (1.4 eq.) followed by 4-chloro-6,7-dimethoxyquinazoline (1.5 eq.) were added and the solution was purged with N2 for 10 mins, then Pd(OAc)2 (5 mol %) was added and temperature was raised slowly to 100 °C. The reaction mixture was stirred under N2 atmosphere at 100 °C for 16 hours. After completion of reaction, the reaction mixture was filtered through a pad of celite, and filtrate was evaporated and purified with silica gel column chromatography by using DCM & MeOH as eluent (0-5%). The product SAPTI012S043/IM1 was confirmed with LCMS; yield 31%.

LCMS: [M+H]=441

Step-2: The intermediate SAPTI012S043/IM1 (1.0 eq.) was dissolved in dry dioxane (0.2M), then B2(Pin)2 (1.4 eq.) KOAc (3.5 eq.), followed by PdCl2(dppf) complex (5 mol %) were added and the reaction mixture purged with N2 for 10 mins, later slowly raise the temperature to 100 °C, and stirred at same temperature for 8 hours. After completion of reaction, the reaction mixture was filtered through a pad of celite, and filtrate was evaporated and purified with silica gel column chromatography by using DCM & MeOH as eluent (0-5%). The product SAPTI012S043 was confirmed with LCMS 1H NMR; yield 40%.

LCMS: [M+H]=406

The characterization data of the compounds are given below:

TABLE 1
1H NMR and LCMS data for compounds of the present invention

| | | |
|---|---|---|
| SAPTI012S001 | 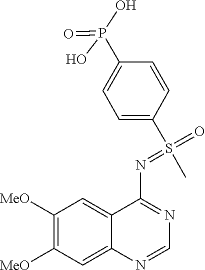 [SMILES: COc1cc2ncnc(N=S(C)(=O)c3ccc(P(=O)(O)O)cc3)c2cc1OC] | LCMS: [M + H] = 424. ¹H-NMR: 8.70 ppm (s, 1H, CH); 8.16 ppm (d, 2H, CH); 7.95 ppm (d, 2H, CH); 7.67 ppm (s, 1H, CH); 7.21 ppm (s, 1H, CH); 3.99 ppm (s, 6H, OCH3); 3.85 ppm (s, 3H, SCH3). |
| SAPTI012S002 | 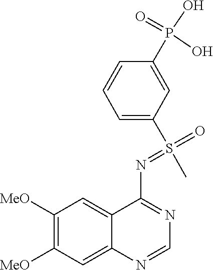 [SMILES: COc1cc2ncnc(N=S(C)(=O)c3cccc(P(=O)(O)O)c3)c2cc1OC] | LCMS: [M + H] = 424. ¹H-NMR: 8.80 ppm (s, 1H, CH); 8.35 ppm (d, 1H, CH); 8.25 ppm (d, 1H, CH); 8.04 ppm (dd, 1H, CH); 7.82 ppm (m, 1H, CH); 7.69 ppm (s, 1H, CH); 7.24 ppm (s, 1H, CH); 4.01 ppm (s, 3H, OCH3); 4.00 ppm (s, 3H, OCH3); 3.92 ppm (s, 3H, SCH3). |
| SAPTI012S003 | 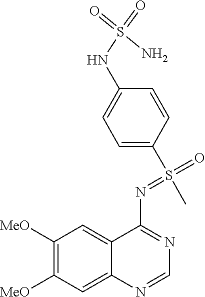 [SMILES: COc1cc2ncnc(N=S(C)(=O)c3ccc(NS(N)(=O)=O)cc3)c2cc1OC] | LCMS: [M + H] = 438. ¹H-NMR: 8.42 ppm (s, 1H, CH); 7.59 ppm (s, 1H, NH); 7.96 ppm (d, 2H, CH); 7.69 ppm (s, 1H, CH); 7.46 ppm (s, 2H, NH2); 7.34 ppm (d, 2H, CH); 7.21 ppm (s, 1H, CH); 3.94 ppm (s, 3H, OCH3); 3.90 ppm (s, 3H, OCH3); 3.69 ppm (s, 3H, SCH3). |
| SAPTI012S004 | 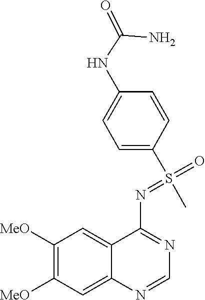 [SMILES: COc1cc2ncnc(N=S(C)(=O)c3ccc(NC(N)=O)cc3)c2cc1OC] | LCMS: [M + H] = 402. ¹H-NMR: 9.72 ppm (s, 1H, NH); 8.36 ppm (s, 1H, CH); 7.89 ppm (d, 2H, CH); 7.67 ppm (d, 2H, CH); 7.57 ppm (s, 1H, CH); 7.15 ppm (s, 1H, CH); 6.27 ppm (s, 2H, NH2); 3.93 ppm (s, 3H, OCH3); 3.92 ppm (s, 3H, OCH3); 3.62 ppm (s, 3H, SCH3). |
| SAPTI012S005 | 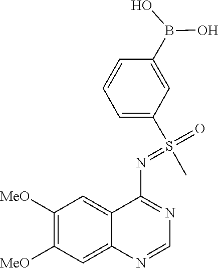 [SMILES: COc1cc2ncnc(N=S(C)(=O)c3cccc(B(O)O)c3)c2cc1OC] | LCMS: [M + H] = 388 ¹H-NMR: 8.50 ppm (s, 1H, CH); 8.42 ppm (s, 2H, B(OH)2); 8.34 ppm (s, 1H, CH); 8.07-8.12 (m, 2H, CH); 7.64 ppm (t, 1H, CH); 7.61 ppm (s, 1H, CH); 7.16 ppm (s, 1H, CH); 3.93 ppm (s, 6H, 2OCH3); 3.67 ppm (s, 3H, SCH3). |
| SAPTI012S006 | 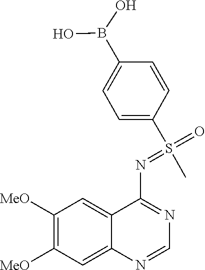 [SMILES: COc1cc2ncnc(N=S(C)(=O)c3ccc(B(O)O)cc3)c2cc1OC] | LCMS: [M + H] = 388 ¹H-NMR: 8.28 ppm (s, 1H, CH); 7.90-8.10 ppm (m, 4H, CH); 7.63 ppm (s, 2H, CH); 7.16 ppm (s, 1H, CH); 3.94 ppm (s, 6H, 2OCH3); 3.64 ppm (s, 3H, SCH3). |
| SAPTI012S007 | 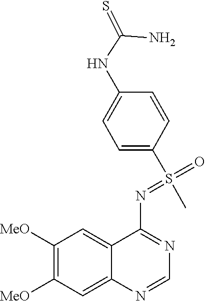 [SMILES: COc1cc2ncnc(N=S(C)(=O)c3ccc(NC(N)=S)cc3)c2cc1OC] | LCMS: [M + H] = 418. ¹H-NMR: 10.11 ppm (s, 1H, NH); 8.30 ppm (s, 1H, CH); 7.97 ppm (d, 2H, CH); 7.84 ppm (d, 2H, CH); 7.57 ppm (s, 1H, CH); 7.16 ppm (s, 1H, CH); 3.92 ppm (s, 6H, 2OCH3); 3.90 ppm (s, 3H, OCH3); 3.69 ppm (s, 3H, SCH3). |
| SAPTI012S008 | 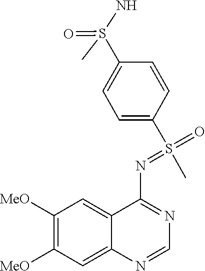 | LCMS: [M + H] = 421 ¹H-NMR: 8.30 ppm (s, 1H, CH); 8.26 ppm (d, 2H, CH); 8.19 ppm (d, 2H, CH); 7.62 ppm (s, 1H, CH); 7.17 ppm (s, 1H, CH); 3.93 ppm (s, 3H, OCH3); 3.92 ppm (s, 3H, OCH3); 3.73 ppm (s, 3H, SCH3); 3.13 ppm (s, 3H, SCH3). |
| SAPTI012S009 | 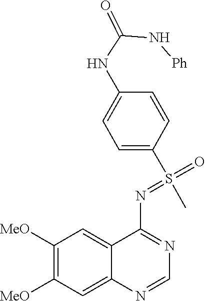 [SMILES: COc1cc2ncnc(N=S(C)(=O)c3ccc(NC(=O)Nc4ccccc4)cc3)c2cc1OC] | LCMS: [M + H] = 478 ¹H-NMR: 9.13 ppm (2, 1H, NH); 8.76 ppm (s, 1H, CH); 8.49 ppm (S, 1H, NH); 8.39 ppm (s, 1H, CH); 7.65 ppm (s, 1H, CH); 7.55-7.65 ppm (m, 3H, CH); 7.44 ppm (d, 2H, CH); 7.30 ppm (t, 2H, CH); 7.17 ppm (s, 1H, CH); 7.01 ppm (t, 1H, CH); 3.94 ppm (s, 3H, OCH3); 3.93 ppm (s, 3H, OCH3); 3.68 ppm (s, 3H, SCH3). |
| SAPTI012S010 | 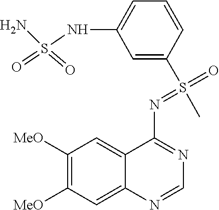 [SMILES: COc1cc2ncnc(N=S(C)(=O)c3cccc(NS(N)(=O)=O)c3)c2cc1OC] | LCMS: [M + H] = 438. ¹H-NMR: 10.0 ppm (s, 1H, NH); 8.38 ppm (s, 1H, CH); 7.90 ppm (t, 1H, CH); 7.55-7.65 ppm (m, 3H, CH); 7.35 ppm (s, 2H, NH2); 7.17 ppm (s, 1H, CH); 3.93 ppm (s, 3H, OCH3); 3.92 ppm (s, 3H, OCH3); 3.68 ppm (s, 3H, SCH3). |
| SAPTI012S011 | 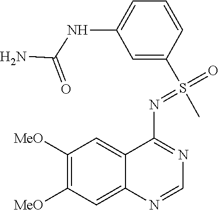 [SMILES: COc1cc2ncnc(N=S(C)(=O)c3cccc(NC(N)=O)c3)c2cc1OC] | LCMS: [M + H] = 402 ¹H-NMR: 9.88 ppm (s, 1H, NH); 8.56 ppm (s, 1H, CH); 8.34 ppm (s, 1H, CH); 7.99 ppm (d, 1H, CH); 7.91 ppm (s, 1H, CH); 7.60-7.66 ppm (m, 2H, CH); 7.23 ppm (s, 1H, CH); 6.45 ppm (s, 1H, NH2); 6.05 ppm (s, 1H, NH2); 3.99 ppm (s, 3H, OCH3); 3.95 ppm (s, 3H, OCH3); 3.37 ppm (s, 3H, SCH3). |
| SAPTI012S012 | 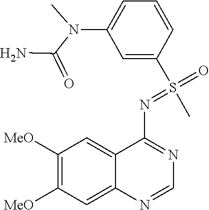 [SMILES: COc1cc2ncnc(N=S(C)(=O)c3cccc(N(C)C(N)=O)c3)c2cc1OC] | LCMS: [M + H] = 416 ¹H-NMR: 8.39 ppm (s, 1H, CH); 8.05 ppm (s, 1H, CH); 7.80 ppm (d, 1H, CH); 7.56-7.63 ppm (m, 2H, CH); 7.56 ppm (s, 1H, CH); 7.17 ppm (s, 1H, CH); 6.25 ppm (s, 2H, NH2); 3.93 ppm (s, 3H, OCH3); 3.92 ppm (s, 3H, OCH3); 3.71 ppm (s, 3H, SCH3); 3.23 ppm (s, 3H, NCH3). |
| SAPTI012S013 | 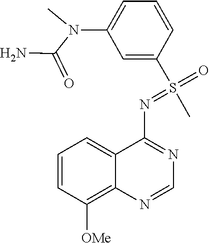 [SMILES: COc1cccc2c(N=S(C)(=O)c3cccc(N(C)C(N)=O)c3)ncnc12] | LCMS: [M + H] = 386 ¹H-NMR: 9.15 ppm (s, 1H, CH); 8.47 ppm (s, 1H, CH); 8.25 ppm (d, 1H, CH); 7.91 ppm (d, 1H, CH); 7.67 ppm (d, 1H, CH); 7.47-7.55 ppm (m, 2H, CH); 7.31 ppm (d, 1H, CH); 6.07 ppm (s, 2H, NH2); 3.92 ppm (s, 3H, OCH3); 3.65 ppm (s, 3H, SCH3); 3.17 ppm (s, 3H, NCH3). |
| SAPTI012S014 | 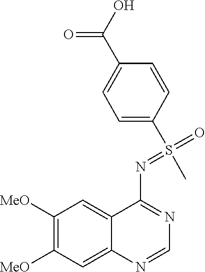 [SMILES: COc1cc2ncnc(N=S(C)(=O)c3ccc(C(=O)O)cc3)c2cc1OC] | LCMS: [M + H] = 388. ¹H-NMR: 8.30 ppm (s, 1H, CH); 8.05-8.20 ppm (m, 4H, CH); 7.58 ppm (m, 1H, CH); 7.16 ppm (s, 1H, CH); 3.92 ppm (s, 3H, OCH3); 3.90 ppm (s, 3H, OCH3); 3.70 ppm (s, 3H, SCH3). |
| SAPTI012S015 | 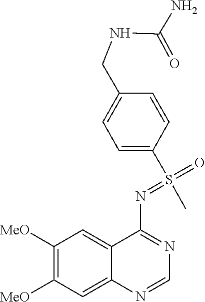 [SMILES: COc1cc2ncnc(N=S(C)(=O)c3ccc(CNC(N)=O)cc3)c2cc1OC] | LCMS: [M + H] = 416. ¹H-NMR: 8.33 ppm (s, 1H, CH); 8.00 ppm (d, 2H, CH); 7.57 ppm (s, 1H, CH); 7.51 ppm (d, 1H, CH); 7.16 ppm (s, 1H, CH); 6.58 ppm (d, 1H, NH); 5.62 ppm (s, 2H, NH2); 4.27 ppm (d, 2H, CH2); 3.92 ppm (s, 6H, 2OCH3); 3.74 ppm (s, 3H, SCH3). |
| SAPTI012S016 | 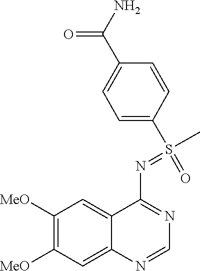 [SMILES: COc1cc2ncnc(N=S(C)(=O)c3ccc(C(N)=O)cc3)c2cc1OC] | LCMS: [M + H] = 387 ¹H-NMR: 8.30 ppm (s, 1H, CH); 8.21 ppm (s, 1H, NH); 8.12 ppm (d, 2H, CH); 8.08 ppm (d, 2H, CH); 7.65 ppm (s, 1H, NH); 7.58 ppm (s, 1H, CH); 7.16 ppm (s, 1H, CH); 3.93 ppm (s, 3H, OCH3); 3.92 ppm (s, 3H, OCH3); 3.71 ppm (s, 3H, SCH3). |
| SAPTI012S017 | 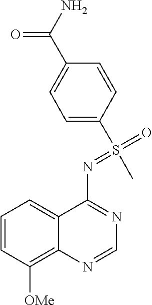 [SMILES: COc1cccc2c(N=S(C)(=O)c3ccc(C(N)=O)cc3)ncnc12] | LCMS: [M + H] = 357 ¹H-NMR: 8.42 ppm (s, 1H, CH); 8.20 ppm (s, 1H, NH); 8.07-8.14 ppm (m, 4H, CH); 7.90 (dd, 1H, CH); 7.65 ppm (s, 1H, NH); 7.51 ppm (t, 1H, CH); 7.32 ppm (dd, 1H, CH); 3.92 ppm (s, 3H, OCH3); 3.73 ppm (s, 3H, SCH3). |
| SAPTI012S018 | 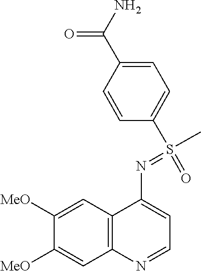 [SMILES: COc1cc2nccc(N=S(C)(=O)c3ccc(C(N)=O)cc3)c2cc1OC] | LCMS: [M + H] = 386 ¹H-NMR: 8.19 ppm (d, 1H, CH); 8.14 ppm (s, 1H, NH); 8.07 ppm (d, 2H, CH); 8.02 ppm (d, 2H, CH); 7.73 ppm (s, 1H, CH); 7.63 ppm (s, 1H, NH); 7.21 ppm (s, 1H, CH); 6.62 ppm (d, 1H, CH); 3.97 ppm (s, 3H, OCH3); 3.89 ppm (s, 3H, OCH3); 3.68 ppm (s, 3H, SCH3). |
| SAPTI012S019 | 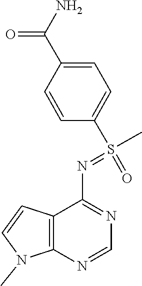 | LCMS: [M + H] = 330 ¹H-NMR: 8.20 ppm (s, 1H, NH); 8.09 ppm (s, 1H, CH); 8.06 ppm (m, 4H, CH); 7.64 ppm (s, 1H, NH); 7.30 ppm (d, 1H, CH); 6.50 ppm (d, 1H, CH); 3.73 ppm (s, 3H, NCH3); 3.63 ppm (s, 3H, SCH3). |
| SAPTI012S020 | 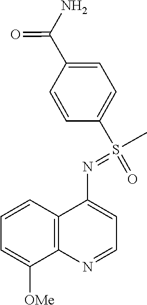 [SMILES: COc1cccc2c(N=S(C)(=O)c3ccc(C(N)=O)cc3)ccnc12] | LCMS: [M + H] = 355 ¹H-NMR: 8.32 ppm (d, 1H, CH); 8.14 ppm (s, 1H, NH); 8.06 ppm (d, 2H, CH); 8.02 ppm (d, 2H, CH); 7.63 ppm (s, 1H, NH); 7.45 ppm (t, 1H, CH); 7.14 ppm (d, 1H, CH); 6.77 ppm (d, 1H, CH); 3.91 ppm (s, 3H, OCH3); 3.70 ppm (s, 3H, SCH3). |
| SAPTI012S021 | 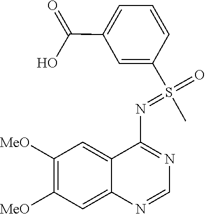 [SMILES: COc1cc2ncnc(N=S(C)(=O)c3cccc(C(=O)O)c3)c2cc1OC] | LCMS: [M + H] = 388. ¹H-NMR: 13-14 ppm (s, broad, 1H, COOH); 8.75 ppm (s, 1H, CH); 8.61 ppm (s, 1H, CH); 8.25- 8.30 ppm (dd, 2H, CH); 7.86 ppm (t, 1H, CH); 7.69 ppm (s, 1H, CH); 7.29 ppm (s, 1H, CH); 3.99 ppm (s, 3H, OCH3); 3.98 ppm (s, 3H, OCH3); 3.92 ppm (s, 3H, SCH3). |
| SAPTI012S022 | 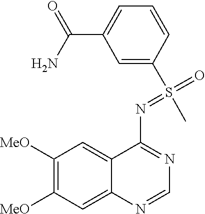 [SMILES: COc1cc2ncnc(N=S(C)(=O)c3cccc(C(N)=O)c3)c2cc1OC] | LCMS: [M + H] = 387. ¹H-NMR: 8.55 ppm (t, 1H, CH); 8.33 ppm (s, 1H, CH); 8.27 ppm (s, 1H, NH); 8.17-8.21 ppm (m, 2H, CH); 7.76 ppm (t, 1H, CH); 7.65 ppm (s, 1H, NH); 7.61 ppm (s, 1H, CH); 7.17 ppm (s, 1H, CH); 3.94 ppm (s, 3H, OCH3); 3.93 ppm (s, 3H, OCH3); 3.72 ppm (s, 3H, SCH3). |
| SAPTI012S023 | 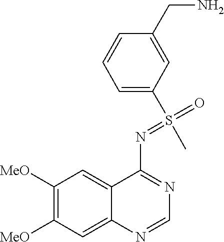 [SMILES: COc1cc2ncnc(N=S(C)(=O)c3cccc(CN)c3)c2cc1OC] | LCMS: [M + H] = 373 ¹H-NMR: 8.35 ppm (s, 1H, CH); 8.14 ppm (s, 1H, CH); 7.91 ppm (d, 1H, CH); 7.70 ppm (d, 1H, CH); 7.62 ppm (d, 1H, CH); 7.60 ppm (s, 1H, CH); 7.16 ppm (s, 1H, CH); 3.93 ppm (s, 3H, OCH3); 3.90 ppm (s, 3H, OCH3); 3.88 ppm (s, 2H, CH2); 3.67 ppm (s, 3H, SCH3); 3.49 ppm (s, 2H, NH2) |
| SAPTI012S024 | 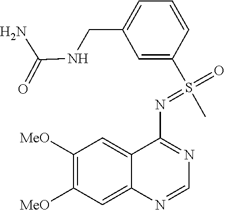 [SMILES: COc1cc2ncnc(N=S(C)(=O)c3cccc(CNC(N)=O)c3)c2cc1OC] | LCMS: [M + H] = 416 ¹H-NMR: 8.33 ppm (s, 1H, CH); 7.98 ppm (s, 1H, CH); 7.87-7.90 ppm (m, 1H, CH); 7.61-7.63 (m, 3H, CH); 7.16 ppm (s, 1H, CH); 6.58 ppm (t, 1H, NH); 5.60 ppm (s, 2H, NH2); 4.31 (d, 2H, CH2); 3.98 ppm (s, 3H, OCH3); 3.94 ppm (s, 3H, OCH3); 3.65 ppm (s, 3H, SCH3). |
| SAPTI012S025 | 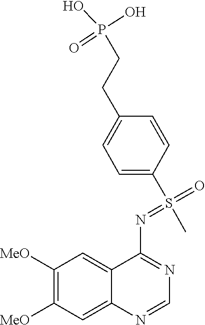 [SMILES: COc1cc2ncnc(N=S(C)(=O)c3ccc(CCP(=O)(O)O)cc3)c2cc1OC] | LCMS: [M + H] = 452. ¹H-NMR: 8.35 ppm (s, 1H, CH); 7.91 ppm (d, 2H, CH); 7.56 ppm (s, 1H, CH); 7.47 ppm (d, 2H, CH); 7.15 ppm (s, 1H, CH); 3.92 ppm (s, 3H, OCH3); 3.91 ppm (s, 3H, OCH3); 3.64 ppm (s, 3H, SCH3); 2.84 ppm (t, 2H, CH2); 1.55 ppm (t, 2H, CH2). |
| SAPTI012S026 | 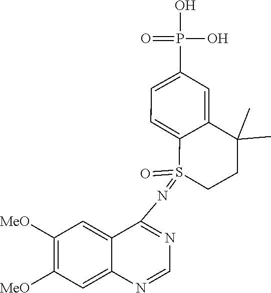 [SMILES: COc1cc2ncnc(N=S3(=O)CCC(C)(C)c4cc(P(=O)(O)O)ccc43)c2cc1OC] | LCMS: [M + H] = 478. ¹H-NMR: 8.39 ppm (s, 1H, CH); 8.15 ppm (d, 1H, CH); 7.88 ppm (d, 1H, NH); 7.65-7.75 ppm (m, 1H, CH); 7.50 ppm (s, 1H, CH); 7.15 ppm (s, 1H, CH); 4.58 ppm (t, 2H, CH2); 3.91 ppm (s, 3H, OCH3); 3.89 ppm (s, 3H, OCH3); 2.44 ppm (t, 2H, CH2); 1.46 ppm (s, 3H, CH3); 1.44 ppm (s, 3H, CH3). |
| SAPTI012S027 | 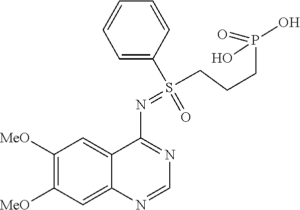 [SMILES: COc1cc2ncnc(N=S(=O)(CCCP(=O)(O)O)c3ccccc3)c2cc1OC] | LCMS: [M + H] = 452. ¹H-NMR: 8.28 ppm (s, 1H, CH); 7.94 ppm (d, 2H, CH); 7.74 ppm (t, 1H, CH); 7.67 ppm (t, 2H, CH); 7.62 ppm (s, 1H, CH); 7.13 ppm (s, 1H, CH); 3.94 ppm (s, 3H, OCH3); 3.93 ppm (s, 3H, OCH3); 3.87 ppm (m, 2H, CH2); 1.96 ppm (m, 1H, CH2); 1.86 ppm (m, 1H, CH2); 1.74 ppm (m, 2H, CH2). |
| SAPTI012S028 | 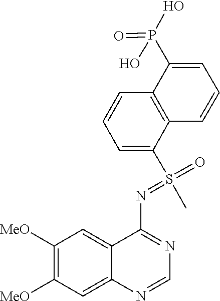 [SMILES: COc1cc2ncnc(N=S(C)(=O)c3cccc4c(P(=O)(O)O)cccc34)c2cc1OC] | LCMS: [M + H] = 474 ¹H-NMR: 9.22 ppm, d, 1H, CH); 8.78 ppm (d, 1H, CH); 8.41 ppm (d, 1H, CH); 8.20 ppm (s, 1H, CH); 8.04 ppm (dd, 1H, CH); 7.77 ppm (t, 1H, CH); 7.63 ppm (s, 1H, CH); 7.5g ppm (m, 1H, CH); 7.15 ppm (s, 1H, CH); 3.86 ppm (s, 3H, OCH3); 3.82 ppm (s, H, OCH3); 3.69 ppm (s, 3H, SCH3). |
| SAPTI012S029 | 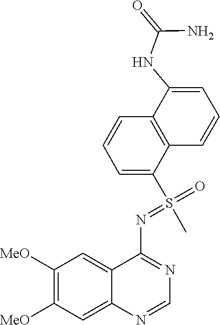 [SMILES: COc1cc2ncnc(N=S(C)(=O)c3cccc4c(NC(N)=O)cccc34)c2cc1OC] | LCMS: [M + H] = 452 ¹H-NMR: 8.75 ppm, (s, 1H, NH); 8.42-8.48 ppm (m, 3H, CH); 8.20 ppm (s, 1H, CH); 7.96 ppm (d, 1H, CH); 7.83 ppm (t, 1H, CH); 7.65 ppm (s, 1H, CH); 7.51-7.53 ppm (t, 1H, CH); 7.12 ppm (s, 1H, CH); 6.18 ppm (s, 2H, NH2); 3.93 ppm (s, 3H, OCH3); 3.90 ppm (s, H, OCH3); 3.75 ppm (s, 3H, SCH3). |
| SAPTI012S030 | 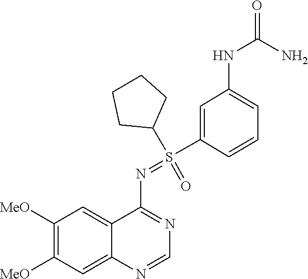 [SMILES: COc1cc2ncnc(N=S(=O)(c3cccc(NC(N)=O)c3)C3CCCC3)c2cc1OC] | LCMS: [M + H] = 456. ¹H-NMR: 8.95 ppm (s, 1H, CH); 8.25 ppm (s, 1H, CH); 8.09 ppm (t, 1H, CH); 7.63 ppm (s, 1H, CH); 7.60 ppm (s, 1H, CH); 7.44 ppm (t, 1H, CH); 7.35 ppm (d, 1H, CH); 7.14 ppm (s, 1H, CH); 5.97 ppm (s, 2H, NH2); 4.14 ppm (pen, 1H, CH); 3.93 ppm (s, 3H, OCH3); 3.92 ppm (s, 3H, OCH3); 2.1-2.25 ppm (m, 2H, CH2); 1.9-2.05 ppm (m, 1H, CH2); 1.75-1.85 ppm (m, 1H, CH2); 1.60-1.75 ppm (m, 4H, CH2). |
| SAPTI012S031 | 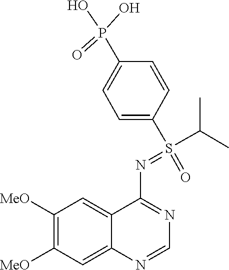 | LCMS: [M + H] = 387 Molecule confirmed with LCMS and purity confirmed with HPLC; HNMR not taken, due to insufficient samples. |
| SAPTI012S032 | 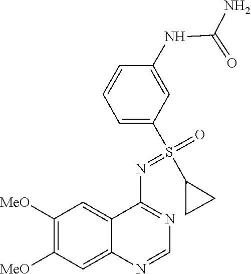 [SMILES: COc1cc2ncnc(N=S(=O)(c3cccc(NC(N)=O)c3)C3CC3)c2cc1OC] | LCMS: [M + H] = 428 ¹H-NMR: 8.97 ppm (s, 1H, CH); 8.29 ppm (s, 1H, CH); 8.13 ppm (s, 1H, NH); 7.60 (s, 1H, CH); 7.57 ppm (d, 1H, CH); 7.40-7.50 ppm (m, 2H, CH); 7.15 ppm (s, 1H, CH); 5.98 ppm (s, 2H, NH2); 3.96 ppm (s, 3H, OCH3); 3.93 ppm (s, 3H, OCH3); 3.05-3.15 ppm (m, 1H, CH); 1.45-1.55 (m, 1H, CH2); 1.25-1.35 (m, 2H, CH2); 1.05-1.15 ppm (m, 1H, CH2). |
| SAPTI012S033 | 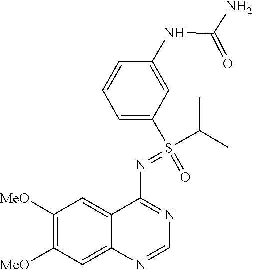 [SMILES: COc1cc2ncnc(N=S(=O)(c3cccc(NC(N)=O)c3)C(C)C)c2cc1OC] | LCMS: [M + H] = 430 ¹H-NMR: 8.96 ppm (s, 1H, NH); 8.28 ppm (s, 1H, CH); 8.08 ppm (t, 1H, CH); 7.65 ppm (d, 1H, CH); 7.63 ppm (s, 1H, CH); 7.47 ppm (t, 1H, CH); 7.34 ppm (d, 1H, CH); 7.16 ppm (s, 1H, CH); 5.96 ppm (s, 2H, NH2); 3.94 ppm (s, 3H, OCH3); 3.93 ppm (s, 3H, OCH3); 3.91 ppm (sep, 1H, CH); 1.41 ppm (d, 3H, CH3); 1.23 ppm (d, 3H, CH3). |
| SAPTI012S034 | 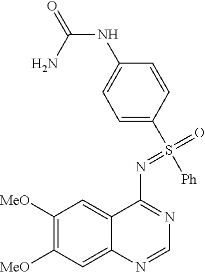 [SMILES: COc1cc2ncnc(N=S(=O)(c3ccccc3)c3ccc(NC(N)=O)cc3)c2cc1OC] | LCMS: [M + H] = 464 ¹H-NMR: 9.11 ppm (s, 1H, NH); 8.31 ppm (s, 1H, CH); 8.02 ppm (d, 2H, CH); 7.96 ppm (d, 2H, CH); 7.77 ppm (s, 1H, CH); 7.55-7.65 ppm (m, 6H, CH); 7.19 ppm (s, 2H, NH2); 4.01 ppm (s, 3H, OCH3); 3.94 ppm (s, 3H, OCH3). |
| SAPTI012S035 | 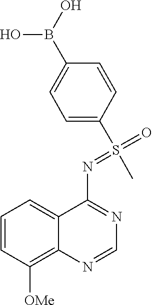 [SMILES: COc1cccc2c(N=S(C)(=O)c3ccc(B(O)O)cc3)ncnc12] | LCMS: [M + H] = 358 ¹H-NMR: 8.43 ppm (d, 1H, CH); 8.05-8.15 ppm (m, 1H, CH); 8.00 ppm (s, 2H, CH); 7.90 ppm (d, 1H, CH); 7.49 ppm (dd, 1H, CH); 7.30 ppm (dd, 1H, CH); 3.91 ppm (s, 3H, OCH3); 3.68 ppm (s, 3H, SCH3). |
| SAPTI012S036 | 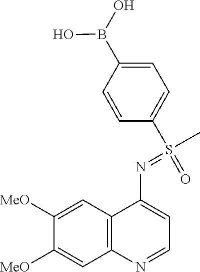 [SMILES: COc1cc2nccc(N=S(C)(=O)c3ccc(B(O)O)cc3)c2cc1OC] | LCMS: [M + H] = 387 ¹H-NMR: 8.23 ppm (d, 1H, CH); 7.93-8.00 ppm (m, 4H, CH); 7.80 ppm (s, 1H, CH); 7.26 (s, 1H, CH); 6.78 ppm (d, 1H, CH); 3.97 ppm (s, 3H, OCH3); 3.92 ppm (s, 3H, OCH3); 3.72 ppm (s, 3H, SCH3). |
| SAPTI012S037 | 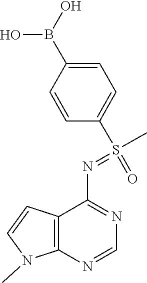 | LCMS: [M + H] = 331 ¹H-NMR: 8.04 ppm (s, 1H, CH); 7.90-7.98 ppm (m, 4H, CH); 7.26 ppm (d, 1H, CH); 6.52 (d, 1H, CH); 3.68 ppm (s, 3H, SCH3); 3.52 ppm (s, 3H, NCH3). |
| SAPTI012S038 | 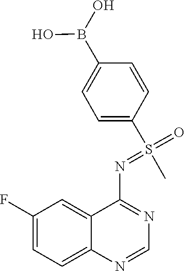 [SMILES: CS(=O)(=Nc1ncnc2ccc(F)cc12)c1ccc(B(O)O)cc1] | LCMS: [M + H] = 346 ¹H-NMR: 8.57 ppm (s, 1H, CH); 8.10 ppm (d, 1H, CH); 7.97-8.03 ppm (m, 4H, CH); 7.87 (d, 1H, CH); 3.72 ppm (s, 3H, SCH3). |
| SAPTI012S039 | 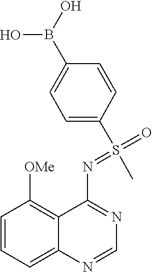 [SMILES: COc1cccc2ncnc(N=S(C)(=O)c3ccc(B(O)O)cc3)c12] | LCMS: [M + H] = 358 ¹H-NMR: 8.42 ppm (d, 1H, CH); 8.14 ppm (d, 2H, CH); 8.03 ppm (d, 2H, CH); 7.78 ppm (s, 1H, CH); 7.31 ppm (d, 1H, CH); 7.09 ppm (d, 2H, CH); 3.96 ppm (s, 3H, OCH3); 3.65 ppm (s, 3H, SCH3). |
| SAPTI012S040 | 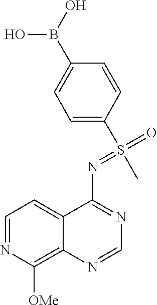 [SMILES: COc1nccc2c(N=S(C)(=O)c3ccc(B(O)O)cc3)ncnc12] | LCMS: [M + H] = 359 ¹H-NMR: 8.55 ppm (d, 1H, CH); 8.12 ppm (d, 1H, CH); 8.03 ppm (d, 1H, CH); 7.85-8.05 ppm (m, 2H, CH); 7.6-7.8 ppm (d, 2H, CH); 4.00 ppm (s, 3H, OCH3); 3.68 ppm (s, 3H, SCH3). |
| SAPTI012S041 | 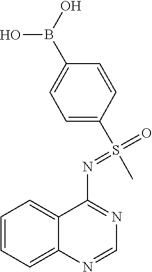 [SMILES: CS(=O)(=Nc1ncnc2ccccc12)c1ccc(B(O)O)cc1] | LCMS: [M + H] = 328 ¹H-NMR: 8.37-8.50 ppm (m, 2H, CH); 8.12 ppm (d, 1H, CH); 7.8-8.10 ppm (m, 4H, CH); 7.77 ppm (s, 1H, CH); 7.60 ppm (d, 1H, CH); 3.70 ppm (s, 3H, SCH3). |
| SAPTI012S042 | 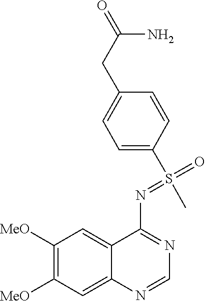 [SMILES: COc1cc2ncnc(N=S(C)(=O)c3ccc(CC(N)=O)cc3)c2cc1OC] | LCMS: [M + H] = 401 ¹H-NMR: 8.34 ppm (s, 1H, CH); 7.98 ppm (d, 2H, CH); 7.60 ppm (s, 1H, NH); 7.58 ppm (s, 1H, CH); 7.54 ppm (d, 2H, CH); 7.16 ppm (t, 1H, CH); 7.00 ppm (s, 1H, NH); 3.92 ppm (s, 6H, 2OCH3); 3.67 ppm (s, 3H, SCH3); 3.52 (s, 2H, CH2). |
| SAPTI0312S043 | 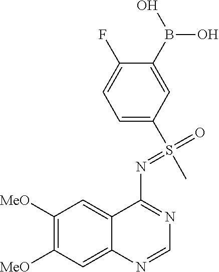 [SMILES: COc1cc2ncnc(N=S(C)(=O)c3ccc(F)c(B(O)O)c3)c2cc1OC] | LCMS: [M + H] = 406 ¹H-NMR: 8.55 ppm (s, 2H, B(OH)2); 8.36 ppm (s, 1H, CH); 8.29-8.32 ppm (m, 1H, CH); 8.09-8.13 ppm (m, 1H, CH); 8.09-8.13 (m, 1H, CH); 7.59 ppm (s, 1H, CH); 7.37 ppm (s, 1H, CH); 7.17 ppm (s, 1H, CH); 3.93 ppm (s, 6H, 2OCH3); 3.82 ppm (s, 3H, SCH3). |

Optical Resolution of Mixtures of (R) and (S) Enantiomers of this Invention:

Mixtures of (R) and (S) enantiomers/racemates of this invention are separated into individual enantiomers by using analytical or/and preparative chiral chromatography (HPLC). The compound of Example 7 was subjected to chiral separation and their activity values are given in Table 3.

### ENPP-1 Inhibition Assay

Assay method: Human ENPP-1 at 3 nM prepared in pH 7.4 was incubated with 5 μM cGAMP substrate with the test samples prepared in buffer with pH 7.4 along with 40 μM HSA. The reaction was incubated at RT for 3 hrs. Post incubation, the reaction was stopped by heating the contents at 95 °C for 10 mins. 10 μl of the solution was added to 384-well plate to which 10 μl of AMP Glo reagent-1 was added and incubated for 60 mins at 25 °C. After the incubation, 20 μl of AMP detection solution was added to each well with the enzyme reaction and incubated for 60 mins at 25 °C. The luminescence signal (RLU) was recorded using SpectraMax I3X plate reader.

The luminescence signal is measured as a function of concentration of the inhibitor. If the inhibitor molecule is active, as the concentration of the inhibitor increases, the luminescence value (referred to as OD) decreases. % Inhibition is then calculated as

% Inhibition=((OD of Control−OD of sample)/OD of Control)×100

In this disclosure, the IC50 value of an inhibitor molecule is measured as the concentration of inhibitor which inhibits growth of 50% of the human ENPP-1. A graph of inhibitor concentration on X-axis vs. percentage inhibition on Y-axis is drawn and the slope is measured as the IC50 value.

Several inventive compounds are subjected to ENPP-1 inhibition assay to identify the IC50 values and/or % inhibition. The metabolic stability of some of these compounds has also been measured.

The results of the ENPP-1 inhibition assay of several compounds are shown in Table 1. From the data presented in Table 1, many lead molecules of the present invention inhibit function of phosphodiesterase enzyme, such as ENPP-1, with promising potency for treatment of diseases such as cancer.

In some aspects, the present invention provides method of treating cancer in an individual in need thereof, wherein the method comprises administering to the individual an effective amount of a compound or salt thereof of the present invention.

In some respects, the present invention provides method of treating a disease or disorder associated with ENPP-1 enzyme in an individual in need thereof, wherein the method comprises administering to the individual an effective amount of a compound or salt thereof of the present invention.

A compound or salt thereof detailed herein, or salt thereof may be formulated for any available delivery route, including an oral, mucosal (e.g., nasal, sublingual, vaginal, buccal or rectal), parenteral (e.g., intramuscular, subcutaneous or intravenous), topical or transdermal delivery form.

TABLE 2
ENPP-1 inhibition
IC50 values of compounds of the invention

| Code | <100 nM | 0.1-1 uM | 1-10 uM | >10 uM |
|---|---|---|---|---|
| SAPTI012S001 | | | Y | |
| SAPTI012S002 | | | Y | |
| SAPTI012S003 | | | | Y |
| SAPTI012S004 | Y | | | |
| SAPTI012S005 | Y | | | |
| SAPTI012S006 | Y | | | |
| SAPTI012S007 | | Y | | |
| SAPTI012S008 | Y | | | |
| SAPTI012S009 | Y | | | |
| SAPTI012S010 | | Y | | |
| SAPTI012S011 | Y | | | |
| SAPTI012S012 | | Y | | |
| SAPTI012S013 | | | Y | |
| SAPTI012S014 | | Y | | |
| SAPTI012S015 | Y | | | |
| SAPTI012S016 | Y | | | |
| SAPTI012S017 | | | | Y |
| SAPTI012S018 | | | Y | |
| SAPTI012S019 | | | Y | |
| SAPTI012S020 | | | Y | |
| SAPTI012S021 | Y | | | |
| SAPTI012S022 | | Y | | |
| SAPTI012S023 | | | | Y |
| SAPTI012S024 | Y | | | |
| SAPTI012S025 | | Y | | |
| SAPTI012S026 | | | | Y |
| SAPTI012S027 | | | | Y |
| SAPTI012S028 | Y | | | |
| SAPTI012S029 | Y | | | |
| SAPTI012S030 | Y | | | |
| SAPTI012S031 | Y | | | |
| SAPTI012S032 | | Y | | |
| SAPTI012S033 | | | | Y |
| SAPTI012S034 | | Y | | |
| SAPTI012S035 | | Y | | |
| SAPTI012S036 | Y | | | |
| SAPTI012S037 | | | | Y |
| SAPTI012S038 | | | | Y |
| SAPTI012S039 | | | Y | |
| SAPTI012S040 | Y | | | |
| SAPTI012S041 | | | | Y |
| SAPTI012S042 | Y | | | |
| SAPTI012S043 | | | Y | |

One of the enantiomers of compound of Formula I is less active than the other. The codes SAPTI012S006-E1 and SAPTI012S006-E2 refer to the two enantiomers of SAPTI012S006. The enantiomers are separated by chiral HPLC. The enantiomers are yet to be assigned the Cahn Ingold Prelog configurations (R or S).

TABLE 3
ENPP-1 inhibition
IC50 values of enantiomers of SAPTI012S006

| Code | Nature of sample | IC50 value range |
|---|---|---|
| SAPTI012S006 | racemic | <100 nM |
| SAPTI012S006-E1 | enantiomer-E1 | >100 nM |
| SAPTI012S006-E2 | enantiomer-E2 | <100 nM |

In another embodiment of the invention, there is provided a use of a compound of Formula I in the manufacture of a medicament for use in the treatment of cancer. In another embodiment of the invention, there is provided a pharmaceutical composition comprising a pharmaceutically acceptable diluent and a therapeutically effective amount of a compound as defined in Formula I.

In one embodiment, the pharmaceutical formulation containing a compound of Formula I or a salt thereof is a formulation adapted for parenteral administration. In another embodiment, the formulation is a long-acting parenteral formulation. In a further embodiment, the formulation is a nano-particle formulation.

In one embodiment, the pharmaceutical formulation containing a compound of Formula I or a salt thereof is a formulation adapted for oral, rectal, topical or intravenous formulation, wherein the pharmaceutical formulation optionally comprises any one or more of a pharmaceutically acceptable carrier, adjuvant or vehicle.

A compound as represented by Formula I or salt thereof may be formulated with suitable carriers to provide delivery forms that include, but are not limited to, tablets, caplets, capsules, cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

In one embodiment, the compounds of Formula I are formulated for oral administration, and can be administered as a conventional preparation, for example, as any dosage form of a solid agent such as tablets, powders, granules, capsules and the like; an aqueous agent; an oily suspension; or a liquid agent such as syrup and elixir. In one embodiment, the compounds of Formula I are formulated for parenteral administration and can be administered as an aqueous or oily suspension injectable, or a nasal drop. Upon preparation of a parenteral formulation with a compound of Formula I, conventional excipients, binders, lubricants, aqueous solvents, oily solvents, emulsifiers, suspending agents, preservatives, stabilizers and the like may be arbitrarily used.

For instance, for oral administration in the form of a tablet or capsule, the compound of Formula I can be combined with an oral, non-toxic pharmaceutically acceptable inert carrier such as ethanol, glycerol, water and the like. Powders are prepared by comminuting the compound of Formula Ito a suitable fine size and mixing with a similarly comminuted pharmaceutical carrier such as an edible carbohydrate, as, for example, starch or mannitol. Flavoring, preservative, dispersing and colouring agents can also be present.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and colouring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an alginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free-flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solutions, syrups and elixirs can be prepared in dosage unit form so that a given quantity contains a predetermined amount of the compound. Syrups can be prepared by dissolving the compound in a suitably flavoured aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavour additive such as peppermint oil or natural sweeteners or saccharin or other artificial sweeteners, and the like can also be added.

Where appropriate, dosage unit formulations for oral administration can be microencapsulated. The formulations of compounds of Formula I can also be prepared to prolong or sustain the release of the compound, as for example by coating or embedding particulate material in polymers, wax or the like.

The compounds of Formula I or salts, solvates or hydrates thereof, can also be administered in the form of liposome delivery systems, such as small unilamellar vesicles, large unilamellar vesicles and multilamellar vesicles. Liposomes can be formed from a variety of phospholipids, such as cholesterol, stearyl amine or phosphatidylcholines.

The compounds of Formula I or salts, solvates or hydrates thereof, may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropyl-methacrylamidephenol, polyhydroxyethylaspartamide-phenol, or poly-ethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period. For example, the compounds of Formula I may be delivered from a patch by iontophoresis as described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e., by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurized aerosols, nebulizers or insufflators.

Pharmaceutical formulations adapted for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

In addition to the ingredients particularly mentioned above, the formulations described herein may include other agents conventional in the art having regard to the type of formulation in question, for example those suitable for oral administration may include flavouring agents.

A therapeutically effective amount of a compound of Formula I will depend upon a number of factors including, for example, the age and weight of the human or other animal, the precise condition requiring treatment and its severity, the nature of the formulation, and the route of 3administration, and will ultimately be at the discretion of the attendant physician or veterinarian. An effective amount of a salt or hydrate thereof may be determined as a proportion of the effective amount of the compound of Formula I or salts, solvates or hydrates thereof per se.

Embodiments of the present invention provide administration of a compound of Formula I to a healthy or a patient with cancer disease, either as a single agent or in combination with (a) another agent that is effective in cancer disease (b) another agent that improves immune response and robustness, or (c) another agent that reduces inflammation and/or pain.

Even though the foregoing invention has been described in some detail by way of illustration and examples for purposes of clarity of understanding, it is readily apparent to those skilled in the art that certain changes and modifications may be made thereto without departing from the spirit or scope of the appended claims. The preceding merely illustrates the principles of the invention. All the examples and conditional language recited herein are principally intended to aid the reader in understanding the principles of the invention and the concepts contributed by the inventors to furthering the art and are to be construed as being without limitation to such specifically recited examples and conditions. The scope of the present invention, therefore, is not intended to be limited to the exemplary embodiments shown and described herein.

We claim:

1. A compound of Formula I or a pharmaceutically acceptable salt, hydrate, solvate, tautomer, or isomer thereof, 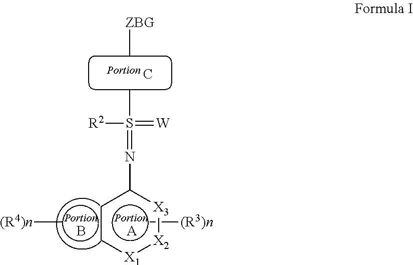
wherein the Formula I comprises portions A and B together constituting tail part, a portion C constituting core part, a sulfoximine-type group R2-S(═W)(═N) which links the tail part with the core part and a zinc binding group (ZBG), where,
the portion A is a six-membered aryl or heteroaryl, which is optionally substituted with one or more R³ groups and where X₁, X₂ and X₃ is CH, N, CR′ with the proviso that not more than two of X₁, X₂ and X₃ is simultaneously N;
the portion B is C6 aryl or 5-6 membered heteroaryl, optionally substituted with one or more R4 groups, wherein the portion B is fused to the portion A where the two shared atoms between the portions B and A come from a pair of carbon atoms or from a pair of atoms where one of the atoms is nitrogen and the other is carbon;
the portion C is selected from the group of structures consisting of (i), (ii), (iii), (iv), (v) and (vi),
where L1 is connected to (i) at variable positions of the ring
where E1 is connected to the sulphur atom of the R2-S(═W)(═N) group and
linker (L1) is connected to “a” atom of the zinc (ZBG) 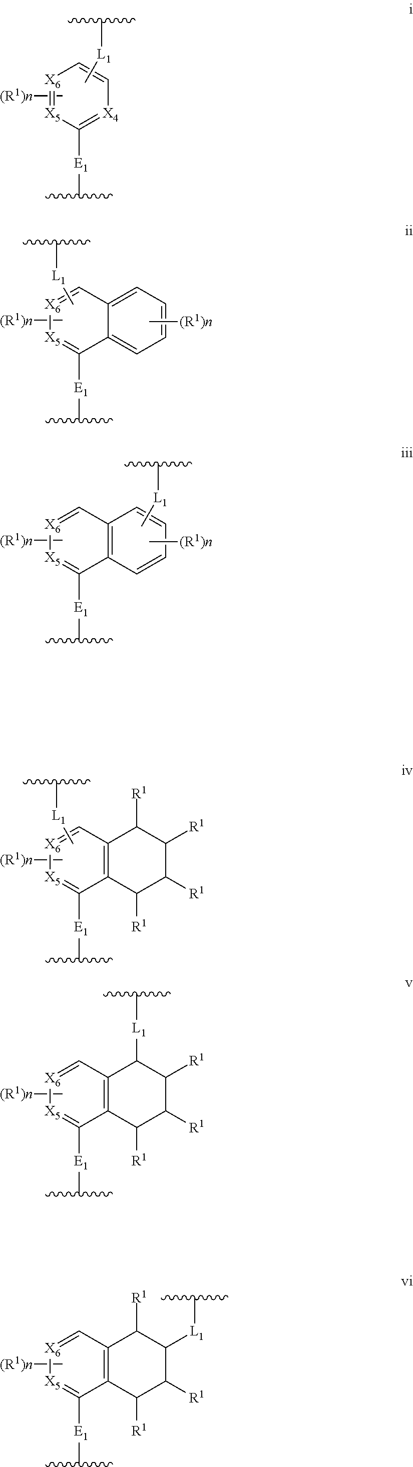
n=0,1,2,3,4 with the proviso that when n>1, the R¹ groups may be the same or different,
R¹, R³, R⁴ groups are selected from the group consisting of R′, halo, OR′, OAr, SR′, SAr, NHAr, NR′R′, CN, SCN, —NHCOR′, COR′, COOR′, COOAr, CF₃, CHF₂, CH₂F, OCF₃, SCF₃ and CH₂Ar, where R′ ═H, CN, C1-6 straight chain alkyl, branched chain alkyl, cycloalkyl, CH₂Ar and where Ar=aryl, substituted aryl, heteroaryl, or substituted hetero aryl;
E1=—(CH₂)_{y}— where y=0, 1 or 2;
L1=—(CH₂)_{y1}, NR″, O or S, C1-6 straight chain alkyl, branched chain alkyl, —(CH₂)_{y1} cycloalkyl, where y1=0,1, 2, or 3;
Wherein R″═H, C1-6 straight chain alkyl, branched chain alkyl, cycloalkyl, aryl, substituted aryl, halogenated alkyl;
X₄, X₅ and X₆═CH, N or CR′;
the portion A is connected to nitrogen atom of the R2-S(═W)(═N) group
where S is chiral sulphur atom,
N is nitrogen,
W is O or NH;
R2 is selected from the group consisting of C1-6 straight, branched, cycloalkyl, alkenyl, alkylene, alkynyl, halo, aryl, heteroaryl, heterocycle, substituted aryl, CF₃, CHF₂, CH₂F, CN and a 2 to 6 membered alkylene group, one end of which is bonded to the sulphur atom and the other end bonded to portion C at the position alpha to the carbon atom of portion C which is bonded to sulphur atom when y=0, forming a cyclic structure and
ZBG as shown in structure (vii) 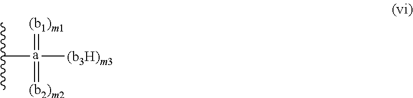
where a=S, P, C, or B atom;
b1, b2=O, S, NH or CH₂,
b3=O, S, NH, CH₂ or NH2O
and m₁, m₂ and m₃=0, 1 with the proviso that only one of m₁, m₂ and m₃ can be 0 at a given instance.

2. The compound as claimed in claim 1, wherein the compound is selected from the group consisting of

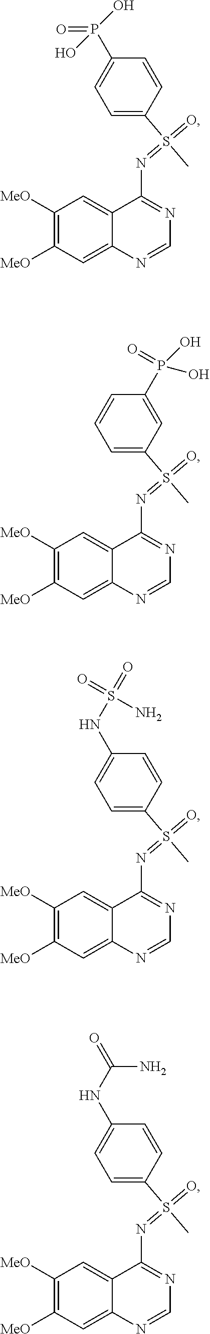 [SMILES: COc1cc2ncnc(N=S(C)(=O)c3ccc(P(=O)(O)O)cc3)c2cc1OC ; COc1cc2ncnc(N=S(C)(=O)c3cccc(P(=O)(O)O)c3)c2cc1OC ; COc1cc2ncnc(N=S(C)(=O)c3ccc(NS(N)(=O)=O)cc3)c2cc1OC ; COc1cc2ncnc(N=S(C)(=O)c3ccc(NC(N)=O)cc3)c2cc1OC]

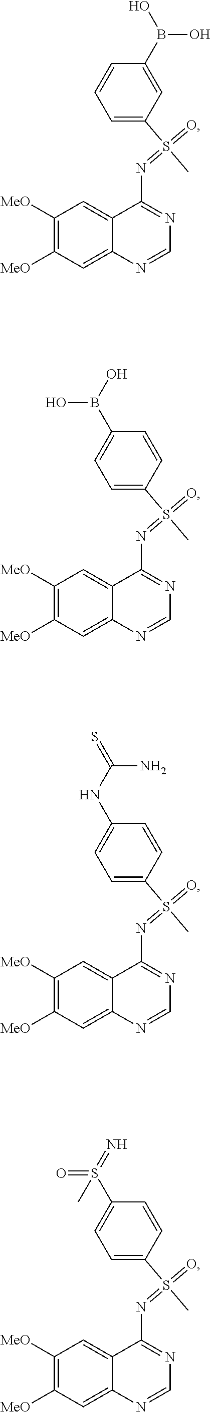 [SMILES: COc1cc2ncnc(N=S(C)(=O)c3cccc(B(O)O)c3)c2cc1OC ; COc1cc2ncnc(N=S(C)(=O)c3ccc(B(O)O)cc3)c2cc1OC ; COc1cc2ncnc(N=S(C)(=O)c3ccc(NC(N)=S)cc3)c2cc1OC ; COc1cc2ncnc(N=S(C)(=O)c3ccc(S(C)(=N)=O)cc3)c2cc1OC]

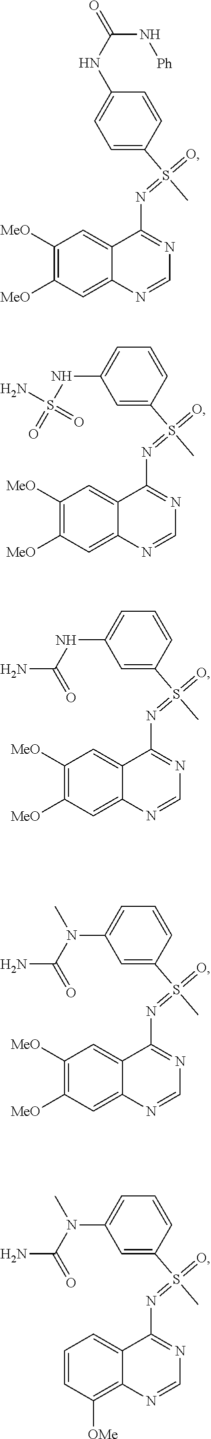 [SMILES: COc1cc2ncnc(N=S(C)(=O)c3ccc(NC(=O)Nc4ccccc4)cc3)c2cc1OC ; COc1cc2ncnc(N=S(C)(=O)c3cccc(NS(N)(=O)=O)c3)c2cc1OC ; COc1cc2ncnc(N=S(C)(=O)c3cccc(NC(N)=O)c3)c2cc1OC ; COc1cc2ncnc(N=S(C)(=O)c3cccc(N(C)C(N)=O)c3)c2cc1OC ; COc1cccc2c(N=S(C)(=O)c3cccc(N(C)C(N)=O)c3)ncnc12]

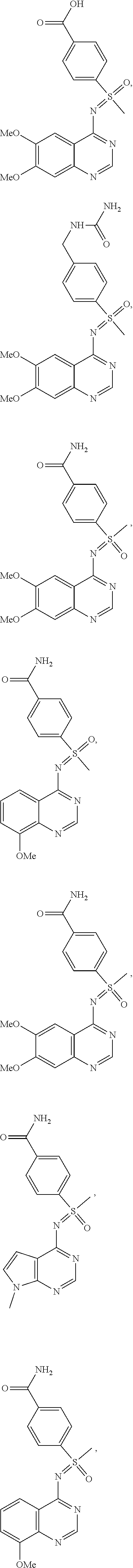

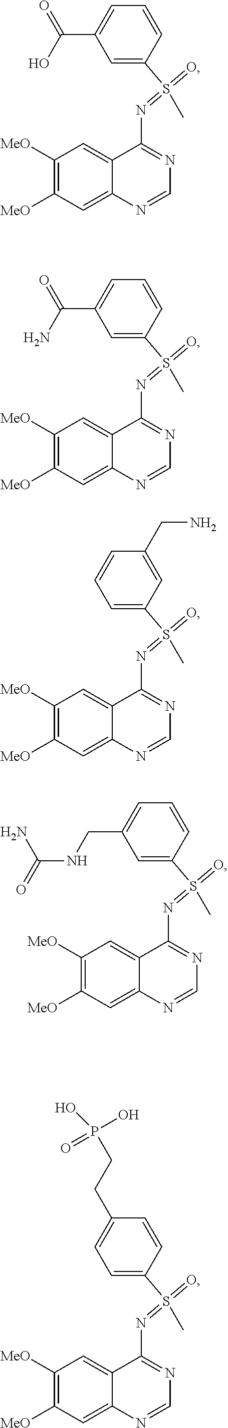

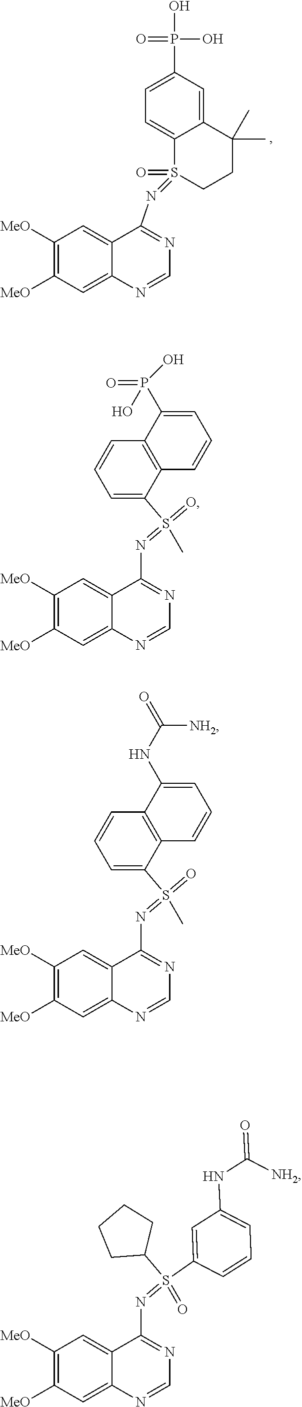 [SMILES: COc1cc2ncnc(N=S3(=O)CCC(C)(C)c4cc(P(=O)(O)O)ccc43)c2cc1OC ; COc1cc2ncnc(N=S(C)(=O)c3cccc4c(P(=O)(O)O)cccc34)c2cc1OC ; COc1cc2ncnc(N=S(C)(=O)c3cccc4c(NC(N)=O)cccc34)c2cc1OC ; COc1cc2ncnc(N=S(=O)(c3cccc(NC(N)=O)c3)C3CCCC3)c2cc1OC]

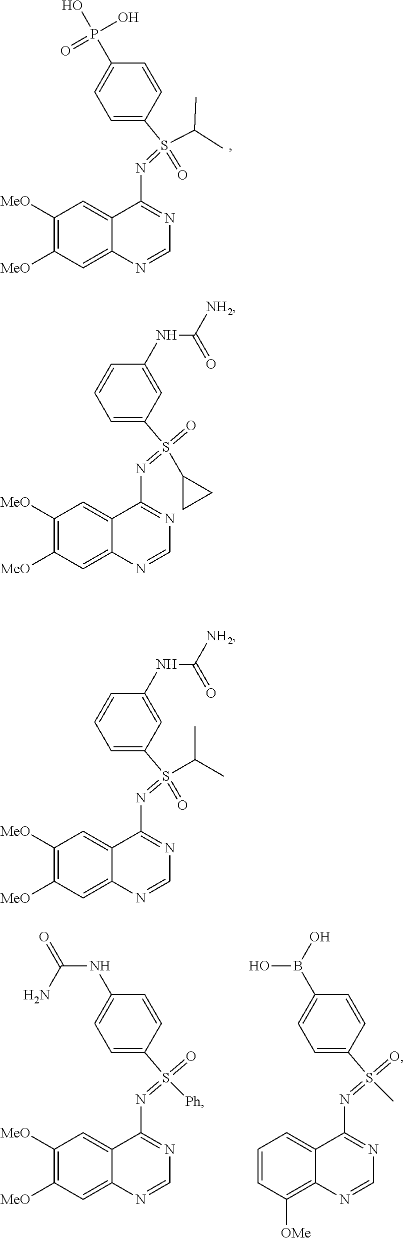

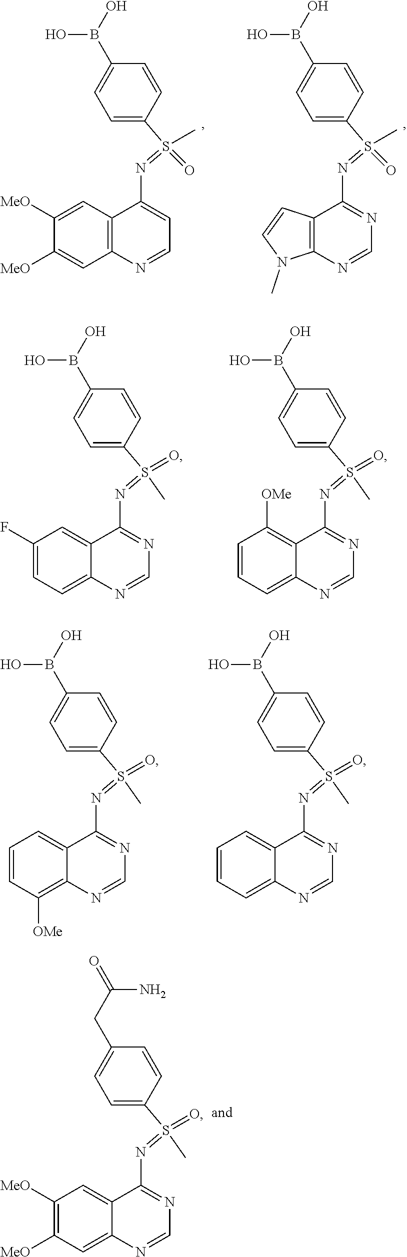

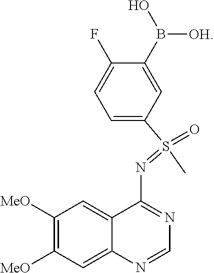 [SMILES: COc1cc2ncnc(N=S(C)(=O)c3ccc(F)c(B(O)O)c3)c2cc1OC]

3. The compounds as claimed in claim 1, wherein the compound inhibits function of phosphodiesterase enzyme.

4. The compound as claimed in claim 3, wherein the phosphodiesterase enzyme is selected from a group consisting of ENPP-1, cyclic nucleotide phosphodiesterase, phospholipases C and D, autotaxin, sphingomyelin phosphodiesterase, DNases, RNases, restriction endonucleases, small-molecule phosphodiesterases and a combination thereof.

5. The compound as claimed in claim 1, wherein the isomer is a stereoisomer of the Formula I.

6. The compound as claimed in claim 5 wherein the stereoisomer is selected from a group consisting of enantiomers and racemates.

7. A pharmaceutical composition comprising a compound of any one of claims 1-6 and a pharmaceutically acceptable carrier or excipient and/or diluent.

8. A method of treating a glioma, glioblastoma multiforme, paraganglioma, supratentorial primordial neuroectodermal tumours, acute myeloid leukemia (AML), prostate cancer, thyroid cancer, colon cancer, chondrosarcoma, cholangiocarcinoma, peripheral T-cell lymphoma, melanoma, intrahepatic cholangiocarcinoma (IHCC), myelodysplastic syndrome (MDS), myeloproliferative disease (MPD), other solid tumours and mycobacterial diseases, which comprises administering to a human in need thereof, a pharmaceutical composition according to claim 7.
